(19) United States
(12) Patent Application Publication
(10) Pub. No.: US 2023/0399374 A1
(43) Pub. Date: Dec. 14, 2023

(54) Compounds, Compositions and Methods of Use to Treat Bone Fractures

(71) Applicant: PURDUE RESEARCH FOUNDATION, West Lafayette, IN (US)

(72) Inventors: Philip Stewart LOW, West Lafayette, IN (US); Stewart A. LOW, West Lafayette, IN (US); Jeffrey Jay Howard NIELSEN, West Lafayette, IN (US)

(21) Appl. No.: 18/033,665

(22) PCT Filed: Aug. 26, 2021
(86) PCT No.: PCT/US2021/047824

§ 371 Date: Apr. 25, 2023

Related U.S. Application Data
(60) Provisional application No. 63/193,748, filed on May 27, 2021.
(60) Provisional application No. 63/105,669, filed on Oct. 26, 2020.

(51) Int. Cl. C07K 14/635; A61K 38/28; C07K 7/04; A61P 19/10

(52) U.S. Cl. CPC C07K 14/635; A61K 38/28; C07K 7/04; A61P 19/10

(57) ABSTRACT

Osteotropic ligand-bone anabolic agent compounds and related compositions and methods of use to treat bone fractures.

### PRIORITY

This patent application is related to and claims the priority benefit of: (a) U.S. Provisional Patent Application No. 63/105,669, filed Oct. 26, 2020, and (b) U.S. Provisional Patent Application No. 63/193,748, filed May 27, 2021. The contents of the aforementioned priority applications are hereby incorporated by reference in their entireties.

### STATEMENT OF GOVERNMENT SUPPORT

This invention was made with government support under DE 028713 awarded by the National Institutes of Health. The government has certain rights in the invention.

### TECHNICAL FIELD

The present disclosure relates to osteotropic ligands, bone anabolic agents, conjugates comprising both, compositions comprising the same, and methods of use to treat bone fractures.

### BRIEF DESCRIPTION OF THE SEQUENCE LISTING

The sequences described herein are set forth in the Figures and also provided in computer-readable form submitted herewith and incorporated herein by reference. The information recorded in computer readable form is identical to the written Sequence Listing provided herein, pursuant to 37 C.F.R. § 1.821(f).

### BACKGROUND

More than 18.3 million bone fractures occur each year in the United States. While some fractures may lead to compromised physical activity, loss of productivity, and decreased quality of life, nonunion fractures can amplify these morbidities by greatly prolonging the time to recovery. Craniofacial fractures can be especially debilitating due to concomitant difficulties with eating and speaking. Delayed hip fracture healing in the elderly can in many cases result in premature mortality. Taken together, the total financial impact of broken bones on reparative costs, convalescent expenses, and physical therapies is estimated at $45.8 billion, and these expenses are anticipated to increase as our population continues to age.

Provided in some embodiments herein are compositions and methods for treating or improving healing of bone fractures (e.g., through the combined use of osteotropic ligands and bone anabolic agents (e.g., conjugates)). This and other objects and advantages, as well as inventive features, will be apparent from the description provided herein.

### SUMMARY

In some embodiments, provided herein is a method for treating a bone-healing event (e.g., bone fracture) of an individual (e.g., in need thereof) comprising administering (e.g., a therapeutically effective amount of) a compound or pharmaceutically acceptable salt thereof provided herein, such as, for example, a compound or pharmaceutically acceptable salt thereof that comprises a bone targeting agent (e.g., an osteotropic ligand) and/or an anabolic agent (e.g., a bone anabolic agent).

In some embodiments, provided herein is a compound having a structure of Formula (I):

X—Y—Z.

In some embodiments, the compound having the structure of Formula (I) is a pharmaceutically acceptable salt thereof. In some embodiments, X is a bone anabolic agent. In some embodiments, X is a bone anabolic agent selected from the group consisting of a parathyroid hormone (PTH) (e.g., or a derivative or fragment thereof (e.g., having bone anabolic activity)), a PTH-related protein (PTHrP) (e.g., or a derivative or fragment thereof (e.g., having bone anabolic activity)), and abaloparatide (e.g., or a derivative or fragment thereof (e.g., having bone anabolic activity)). In some embodiments, Y is absent or a linker (e.g., a releasable linker or a non-releasable linker). In some embodiments, Y is a releasable linker or a non-releasable linker. In some embodiments, Z is an osteotropic ligand (e.g., an acidic oligopeptide (AOP) (e.g., comprising at least 4 amino acid residues (e.g., 4 to 20 amino acid residues))).

In some embodiments, X is a bone anabolic agent selected from the group consisting of a PTH (e.g., or a derivative or fragment thereof (e.g., having bone anabolic activity)), a PTHrP (e.g., or a derivative or fragment thereof (e.g., having bone anabolic activity)), and abaloparatide (e.g., or a derivative or fragment thereof (e.g., having bone anabolic activity)).

In some embodiments, the bone anabolic agent is a PTH or a PTHrP or a derivative or fragment thereof (e.g., (SEQ ID NO: 1 and/or having bone anabolic activity)). In some embodiments, the bone anabolic agent is a parathyroid hormone (PTH) (e.g., or a derivative or fragment thereof). In some embodiments, the bone anabolic agent is a PTHrP or a derivative or fragment thereof). In some embodiments, the bone anabolic agent is a modified PTH or a derivative or fragment thereof). For example, the modified PTH or derivative or fragment thereof is synthetically modified. In some embodiments, the bone anabolic agent is a modified PTHrP or a derivative or fragment thereof such as, for example, comprising SEQ ID NO: 1. In certain embodiments, the modified PTHrP or a derivative or fragment thereof is synthetically modified. In some embodiments, the bone anabolic agent is abaloparatide (e.g., or a derivative or fragment thereof (e.g., having bone anabolic activity)). In some embodiments, the bone anabolic agent is abaloparatide (SEQ ID NO: 2). In some embodiments, the bone anabolic agent is a (e.g., synthetically) modified abaloparatide.

In some embodiments, X is a PTH or a derivative or fragment thereof (e.g., having bone anabolic activity).

In some embodiments, X is a PTHrP or a derivative or fragment thereof (e.g., having bone anabolic activity).

In some embodiments, X is abaloparatide or a derivative or fragment thereof (e.g., having bone anabolic activity).

In some embodiments, X is dasatinib.

In some embodiments, X is proinsulin-like growth factor II (pro-IGF-II).

In some embodiments, X is a cyclic peptide (e.g., optionally substituted 101 or optionally substituted 102). In some embodiments, X is optionally substituted 101. In some embodiments, X is optionally substituted 102. In some embodiments, X is 101. In some embodiments, X is 102.

In some embodiments, X modulates integrin alpha 5 beta 1 activity. In some embodiments, X is a ligand of integrin alpha 5 beta 1. In some embodiments, 101 and 102 modulate integrin alpha 5 beta 1 activity.

In some embodiments, Z is a tetracycline, a phosphonate (e.g., monobisphosphonate, tribisphosphonate, or a polybisphosphonate), or an AOP. In some embodiments, Z is a tetracycline. In some embodiments, Z is a monobisphosphonate, a tribisphosphonate, or a polybisphosphonate. In some embodiments, Z is a monobisphosphonate. In some embodiments, Z is a tribisphosphonate. In some embodiments, Z is a polybisphosphonate.

In some embodiments, Z is a linear chain of amino acid residues. In some embodiments, Z is a branched chain of amino acid residues. In some embodiments, Z is an AOP (e.g., comprising at least 4 glutamic acid amino acid residues or 4 aspartic acid amino acid residues).

In some embodiments, Z comprises at least 4 amino acid residues (e.g., 4 or more, 10 or more, 20 or more, 30 or more, 50 or more, 75 or more, or 100 or more). In some embodiments, Z comprises 4 to 75 acidic amino acid residues (e.g., D-glutamic acid amino acid residues). In some embodiments, Z comprises at most 100 amino acid residues (e.g., 100 or less, 75 or less, 50 or less, 30 or less, 20 or less, 10 or less, or 4 or less). In some embodiments, Z comprises not less than 4 and not more than 35 amino acids. In some embodiments, Z comprises not less than 4 and not more than 20 amino acids. In some embodiments, Z comprises not less than 6 and not more than 30 amino acids. In some embodiments, Z comprises not less than 8 and not more than 30 amino acids. In some embodiments, Z comprises not less than 8 and not more than 20 amino acids. In some embodiments, Z comprises glutamic acid amino acid residues. In some embodiments, Z comprises D-glutamic acid amino acid residues.

In some embodiments, Z comprises 4 to 75 D-glutamic acid amino acid residues. In some embodiments, Z comprises 8 to 30 D-glutamic acid amino acid residues. In some embodiments, Z comprises 8 to 20 D-glutamic acid amino acid residues.

In some embodiments, the AOP the comprises from about 4 to about 20 amino acid residues (such as 4 to about 20 or about 4 to 20) or more amino acid residues, such as 4, 5, 6, 7, 8, 9, 10, 11, 12, 13, 14, 15, 16, 17, 18, 19 or 20. In various embodiments, the AOP comprises about 20 amino acid residues, such as 20 amino acid residues.

In some embodiments, Z comprises at least 4 (e.g., D-) glutamic acid amino acid residues (e.g., 4 to 20 D-glutamic acid amino acid residues) and/or at least 4 (e.g., D-) aspartic acid amino acid residues (e.g., 4 to 20 D-aspartic acid amino acid residues).

In some embodiments, the amino acid is aspartic acid (represented by the letter D), glutamic acid (represented by the letter E), or a mixture thereof. The amino acid residues can have D chirality, L chirality, or a mixture thereof. In some embodiments, the amino acid residue has D chirality. In some embodiments, the amino acid residue has L chirality. In some embodiments, Z comprises at least 4 (e.g., acidic) amino acid residues (e.g., having the same chirality (e.g., D- or L-amino acid residues)). In some embodiments, each of the at least 4 (e.g., acidic) amino acid residue has D chirality. In some embodiments, the aspartic acid is D-aspartic acid or L-aspartic acid. In some embodiments, the glutamic acid is D-glutamic acid or L-glutamic acid. In some embodiments, Z comprises not less than 4 and not more than 20 D-glutamic acid residues or L-glutamic acid residues. In some embodiments, Z comprises not less than 4 and not more than 20 D-aspartic acid residues or L-aspartic acid residues.

In some embodiments, Z comprises at least 4 (e.g., D-) glutamic acid amino acid residues (e.g., 4 to 20 D-glutamic acid amino acid residues) and/or at least 4 (e.g., D-) aspartic acid amino acid residues (e.g., 4 to 20 D-aspartic acid amino acid residues).

In some embodiments, Z comprises a mixture of (e.g., D-) glutamic acid amino acid residues and (e.g., D-) aspartic acid amino acid residues.

In some embodiments, Z comprises at least 4 repeating D-glutamic acid amino acid residues (e.g., 4 repeating D-glutamic acid amino acid residues (DE4) or more, 6 repeating D-glutamic acid amino acid residues (DE6) or more, 8 repeating D-glutamic acid amino acid residues (DE8) or more, 10 repeating D-glutamic acid amino acid residues (DE10) or more, 15 repeating D-glutamic acid amino acid residues (DE15) or more, 20 repeating D-glutamic acid amino acid residues (DE20) or more, 25 repeating D-glutamic acid amino acid residues (DE25) or more, 30 repeating D-glutamic acid amino acid residues (DE30) or more, or 35 repeating D-glutamic acid amino acid residues (DE35) or more). In some embodiments, Z comprises at least DE10 or more, DE15 or more, or DE20 or more). In some embodiments, Z is DE10 or DE20.

In at least some embodiments, Z comprises at least DE15 or at least DE20.

In some embodiments, X is abaloparatide or a derivative or fragment thereof (e.g., having bone anabolic activity) and Z is DE20.

In some embodiments, Y is a non-releasable linker. In some embodiments, Y is a non-releasable linker containing at least one carbon-carbon bond. In some embodiments, Y is a non-releasable linker containing at least one amide bond. In some embodiments, Y is a non-releasable linker containing at least one carbon-carbon bond and at least one amide bond.

In some embodiments, Y is a non-releasable linker and comprises one or more amide bond(s). In some embodiments, Y is a non-releasable linker and comprises 1-20 amide bond(s). In some embodiments, Y is a non-releasable linker and comprises 1-10 amide bond(s). In some embodiments, Y is a non-releasable linker and comprises 10-20 amide bond(s). In some embodiments, Y is a non-releasable linker and comprises 1-5 amide bond(s).

In some embodiments, Y is a non-releasable linker and comprises one or more amino acid linker group(s). In some embodiments, Y is a polypeptide. In some embodiments, the polypeptide comprises 1-20 amino acid residue(s). In some embodiments, the polypeptide comprises 1-10 amino acid residue(s). In some embodiments, the polypeptide comprises 10-20 amino acid residue(s). In some embodiments, the polypeptide comprises 1-5 amino acid residue(s).

In some embodiments, Y is a non-releasable linker and comprises one or more ether bond(s) (C—O). In some embodiments, Y is a non-releasable linker and comprises 1-20 ether bond(s) (C—O). In some embodiments, Y is a non-releasable linker and comprises 1-10 ether bond(s) (C—O). In some embodiments, Y is a non-releasable linker and comprises 10-20 ether bond(s) (C—O). In some embodiments, Y is anon-releasable linker and comprises 1-5 ether bond(s) (C—O).

In some embodiments, Y is a non-releasable linker and comprises one or more polyethylene glycol (PEG) linker group(s). In some embodiments, Y is a PEG.

In some embodiments, Y is a non-releasable linker and comprises one or more thioether bond(s) (C—S). In some embodiments, Y is a non-releasable linker and comprises 1-20 thioether bond(s) (C—S). In some embodiments, Y is a non-releasable linker and comprises 1-10 thioether bond(s) (C—S). In some embodiments, Y is a non-releasable linker and comprises 10-20 thioether bond(s) (C—S). In some embodiments, Y is a non-releasable linker and comprises 1-5 thioether bond(s) (C—S).

In some embodiments, Y is a releasable linker. In some embodiments, Y is a releasable linker containing at least one disulfide (S—S). In some embodiments, Y is a releasable linker containing at least one ester (e.g., O(C═O)). In some embodiments, Y is a releasable linker containing at least one (e.g., protease-specific) amide bond.

In some embodiments, Y is a releasable linker and comprises one or more amide bond(s). In some embodiments, Y is a releasable linker and comprises 1-20 amide bond(s). In some embodiments, Y is a releasable linker and comprises 1-10 amide bond(s). In some embodiments, Y is a releasable linker and comprises 10-20 amide bond(s). In some embodiments, Y is a releasable linker and comprises 1-5 amide bond(s).

In some embodiments, Y is a releasable linker and comprises one or more amino acid linker group(s). In some embodiments, Y is a polypeptide. In some embodiments, the polypeptide comprises 1-20 amino acid residue(s). In some embodiments, the polypeptide comprises 1-10 amino acid residue(s). In some embodiments, the polypeptide comprises 10-20 amino acid residue(s). In some embodiments, the polypeptide comprises 1-5 amino acid residue(s).

In some embodiments, Y is a releasable linker and comprises one or more ether bond(s) (C—O). In some embodiments, Y is a releasable linker and comprises 1-20 ether bond(s) (C—O). In some embodiments, Y is a releasable linker and comprises 1-10 ether bond(s) (C—O). In some embodiments, Y is a releasable linker and comprises 10-20 ether bond(s) (C—O). In some embodiments, Y is a releasable linker and comprises 1-5 ether bond(s) (C—O).

In some embodiments, Y is a releasable linker and comprises one or more PEG linker group(s).

In some embodiments, X is abaloparatide (e.g., or a derivative or fragment thereof (e.g., having bone anabolic activity)), Y is a releasable oligopeptide linker comprising at least one protease-specific amide bond, and Z is 20 repeating DE20.

In some embodiments, X is abaloparatide (e.g., or a derivative or fragment thereof (e.g., having bone anabolic activity)), Y is a non-releasable oligopeptide linker, and Z is DE10. In some embodiments, the compound is SEQ ID NO: 3. In some embodiments, the compound is SEQ ID NO: 14.

In some embodiments, X is abaloparatide or a derivative or fragment thereof (e.g., having bone anabolic activity), Y is a non-releasable oligopeptide linker, and Z is DE20. In some embodiments, the compound is SEQ ID NO: 4.

In some embodiments, X is abaloparatide or a derivative or fragment thereof (e.g., having bone anabolic activity), Y is a non-releasable oligopeptide linker, and Z is DE20. In some embodiments, the compound is SEQ ID NO: 11.

In some embodiments, X is a (poly)peptide. In some embodiments, a compound having the structure of Formula (I) is a (poly)peptide.

In some embodiments, provided herein is a (poly)peptide having bone anabolic activity (e.g., abaloparatide (SEQ ID NO: 2)). In some embodiments, provided herein is a substantially pure (poly)peptide having bone anabolic activity (e.g., abaloparatide), wherein the (poly)peptide comprises an amino acid sequence having at least 75%, at least 85%, at least 95% amino acid sequence identity with an amino acid sequence set forth in SEQ ID NO: 1, SEQ ID NO: 2, SEQ ID NO: 3, SEQ ID NO: 4, or SEQ ID NO: 14. In some embodiments, SEQ ID NO: 3 and/or SEQ ID NO: 14 have bone anabolic activity (e.g., and bone targeting activity). In some embodiments, SEQ ID NO: 4 has bone anabolic activity (e.g., and bone targeting activity). In other embodiments, the (poly)peptide comprises an amino acid sequence having at least 75% sequence identity (e.g., at least 75% sequence identity or more, at least 85% sequence identity or more, at least 90% sequence identity or more, or at least 95% sequence identity or more) with the (e.g., PTH, PTHrP (SEQ ID NO: 1), or abaloparatide (Abalo) (SEQ ID NO: 2)), or the amino acid sequence set forth in SEQ ID NO: 3. In other embodiments, the (poly)peptide comprises an amino acid sequence having at least 75% sequence identity (e.g., at least 75% sequence identity or more, at least 85% sequence identity or more, at least 90% sequence identity or more, or at least 95% sequence identity or more) with PTH or PTHrP (SEQ ID NO: 1), or Abalo (SEQ ID NO: 2)), or the amino acid sequence set forth in SEQ ID NO: 4.

In another embodiment, the (poly)peptide is an amino acid sequence having at least 75% sequence identity or more, at least 85% sequence identity or more, at least 90% sequence identity or more, or at least 95% sequence identity or more to an amino acid sequence shown in FIG. 1A. In another embodiment, the (poly)peptide is an amino acid sequence shown in FIG. 1A. In another embodiment, the (poly)peptide is an amino acid sequence having at least 75% sequence identity or more, at least 85% sequence identity or more, at least 90% sequence identity or more, or at least 95% sequence identity or more to an amino acid sequence shown in FIG. 1B. In another embodiment, the (poly)peptide is an amino acid sequence shown in FIG. 1B. In some embodiments, the (poly)peptide is an amino acid sequence having at least 75% sequence identity or more, at least 85% sequence identity or more, at least 90% sequence identity or more, or at least 95% sequence identity or more to 101 (e.g., see SEQ ID NO: 7 in FIG. 1A). In some embodiments, the (poly)peptide is 101 (e.g., see SEQ ID NO: 7 in FIG. 1A). In some embodiments, the (poly)peptide is an amino acid sequence having at least 75% sequence identity or more, at least 85% sequence identity or more, at least 90% sequence identity or more, or at least 95% sequence identity or more to 102 (e.g., see SEQ ID NO: 8 in FIG. 1B). In some embodiments, the (poly)peptide is 102 (e.g., see SEQ ID NO: 8 in FIG. 1B).

In some embodiments, a compound provided herein comprises a payload. In some embodiments, the payload comprises Abalo or a derivative or fragment thereof (e.g., having bone anabolic activity)) (e.g., SEQ ID NO: 2). In some embodiments, the payload comprises a linker provided herein (e.g., SEQ ID NO: 12). In some embodiments, the payload comprises Abalo or a derivative or fragment thereof (e.g., having bone anabolic activity)) (e.g., SEQ. ID. NO.: 2) and a linker provided herein (e.g., SEQ ID NO: 12).

In some embodiments, the linker is or comprises SEQ ID NO: 12. In some embodiments, the linker is or comprises a polypeptide consisting essentially of SEQ ID NO: 12. In some embodiments, the linker comprises one or more amino acid(s) of SEQ ID NO: 12. In some embodiments, the linker comprises each amino acid of SEQ ID NO: 12. In some embodiments, the linker is SEQ ID NO: 12.

In some embodiments, the (poly)peptide is a pharmaceutically acceptable salt of any compound provided herein (e.g., a compound having a structure of Formula (I), SEQ ID NO: 3, or SEQ ID NO: 4).

In some embodiments, provided herein is a pharmaceutical composition comprising any compound provided herein (e.g., a compound having a structure of Formula (I), SEQ ID NO: 3, or SEQ ID NO: 4), or a pharmaceutically acceptable salt thereof. In certain embodiments, the pharmaceutical composition comprises any compound provided herein and at least one pharmaceutically acceptable carrier or excipient.

In some embodiments, the compound provided herein (e.g., the compound having the structure of Formula (I), SEQ ID NO: 3 or SEQ ID NO: 4) is administered (e.g., subcutaneously) to an individual (e.g., a patient or an individual in need thereof).

Provided in some embodiments herein is a conjugate of formula X—Y—Z.

In some embodiments, X is a bone anabolic agent selected from the group consisting of a PTH, a PTHrP (SEQ ID NO: 1), abaloparatide (SEQ ID NO: 2), a derivative of any of the foregoing having bone anabolic activity, and a fragment of any of the foregoing having bone anabolic activity.

In some embodiments, Y, when present, is a linker, which can be either releasable or non-releasable.

In another embodiment, the linker is an amino acid sequence having at least 75% sequence identity or more, at least 85% sequence identity or more, at least 90% sequence identity or more, or at least 95% sequence identity or more to an amino acid sequence shown in FIG. 1A. In another embodiment, the linker is an amino acid sequence shown in FIG. 1A. In another embodiment, the linker is an amino acid sequence having at least 75% sequence identity or more, at least 85% sequence identity or more, at least 90% sequence identity or more, or at least 95% sequence identity or more to an amino acid sequence shown in FIG. 1B (e.g., SEQ ID NO: 12). In another embodiment, linker is an amino acid sequence shown in FIG. 1B (e.g., SEQ ID NO: 12).

In some embodiments, Z is an osteotropic ligand, which is an AOP comprising at least 11 to 100 amino acid residues.

The amino acid residues can be glutamic acid, aspartic acid, or a mixture thereof. The amino acid residues can have D chirality. The AOP can be a linear chain of amino acid residues. When Y is present, Y can be a non-releasable linker containing at least one carbon-carbon bond and/or at least one amide bond. Alternatively, when Y is present, Y can be a releasable linker containing at least one disulfide, ester, and/or protease-specific amide bond.

In some embodiments, provided herein is a pharmaceutical composition comprising any compound provided herein (e.g., a compound having a structure of Formula (I), SEQ ID NO: 3 or SEQ ID NO: 4), or a pharmaceutically acceptable salt thereof (e.g., and at least one pharmaceutically acceptable carrier or excipient).

Further provided in some embodiments herein is a pharmaceutical composition comprising an effective amount of an above-described conjugate and a pharmaceutically acceptable carrier.

In some embodiments, the compound provided herein (e.g., the compound having the structure of Formula (I), SEQ ID NO: 3 or SEQ ID NO: 4) is administered (e.g., subcutaneously) to an individual (e.g., a patient or an individual in need thereof).

In some embodiments, provided herein is a method of treating a bone fracture (e.g., of an individual (e.g., a patient or an individual in need thereof)). In some embodiments, the method comprises administering (e.g., subcutaneously) a therapeutically effective amount of any compound provided herein (e.g., a compound having a structure of Formula (I), SEQ ID NO: 3 or SEQ ID NO: 4) to the individual (e.g., a patient or an individual in need thereof). In some embodiments, administering (e.g., subcutaneously) the therapeutically effective amount of any compound provided herein (e.g., a compound having a structure of Formula (I), SEQ ID NO: 3 or SEQ ID NO: 4) to the individual (e.g., a patient or an individual in need thereof) treats the bone fracture or improves the healing of the bone fracture of the individual (e.g., a patient or an individual in need thereof).

Provided in some embodiments herein is a method of treating a bone fracture in a patient (e.g., in need thereof), the method comprising administering (e.g., subcutaneously) to the patient (e.g., in need thereof) a therapeutically effective amount of any compound (e.g., having a structure of Formula (I), SEQ ID NO: 3 or SEQ ID NO: 4) or pharmaceutical composition provided herein, thereby treating the bone fracture in the patient (e.g., in need thereof).

In some embodiments, the patient (e.g., in need thereof) is susceptible to a bone fracture. In some embodiments, the patient (e.g., in need thereof) has diabetes mellitus. In some embodiments, the patient (e.g., in need thereof) has osteoporosis. In some embodiments, the patient (e.g., in need thereof) has a maxillofacial deficiency, defect, or injury (e.g., a maxillofacial fracture. In some embodiments, the maxillofacial fracture is a mandibular osteotomy stabilized with a microplate. In some embodiments, the patient (e.g., in need thereof) has diabetes mellitus, osteoporosis, and/or a maxillofacial deficiency, a maxillofacial defect, or a maxillofacial injury (e.g., a maxillofacial fracture). In some embodiments, the patient has one or more comorbidities selected from the group consisting of diabetes mellitus, osteoporosis, a maxillofacial injury, and a maxillofacial deficiency.

In some embodiments, administering (e.g., subcutaneously) the therapeutically effective amount of any compound (e.g., having a structure of Formula (I)) or pharmaceutical composition provided herein is by injection. In some embodiments, administering (e.g., subcutaneously) the therapeutically effective amount of any compound (e.g., having a structure of Formula (I)) or pharmaceutical composition provided herein is by subcutaneous injection. In some embodiments, the therapeutically effective amount of any compound or pharmaceutical composition provided herein is administered by parenterally administration or enterally administration.

In some embodiments, provided herein is a method of treating a bone fracture (e.g., of an individual (e.g., a patient or an individual in need thereof)). In some embodiments, the method comprises administering (e.g., subcutaneously) a therapeutically effective amount of any compound provided herein (e.g., a compound having a structure of Formula (I), SEQ ID NO: 3 or SEQ ID NO: 4) to the individual (e.g., a patient or an individual in need thereof). In some embodiments, administering (e.g., subcutaneously) the therapeutically effective amount of any compound provided herein (e.g., a compound having a structure of Formula (I), SEQ ID NO: 3 or SEQ ID NO: 4) to the individual (e.g., a patient or an individual in need thereof) treats the bone fracture or improves the healing of the bone fracture of the individual (e.g., a patient or an individual in need thereof). In some embodiments, administering the therapeutically effective amount of any compound or pharmaceutical composition provided herein is by parenteral administration or enteral administration.

In some embodiments, administering results in a reduction of pain in the patient within three weeks (e.g., between 2-3 weeks) following administration of the therapeutically effective amount of the compound or pharmaceutical composition provided herein.

Methods for promoting bone growth in a patient (e.g., in need thereof) are also provided. In certain embodiments, the method comprises administering to the patient a therapeutically effective amount of a compound or pharmaceutical composition provided herein, thereby increasing a bone mineral density in a bone of the patient as compared to pre-treatment (e.g., bone density prior to administration of the compound or pharmaceutical composition provided herein).

In some embodiments, the increased bone mineral density in the bone occurs at a fracture site or in one or more resorption pits present on the bone (e.g., in a patient experiencing osteoporosis). In some embodiments of the methods provided herein, a compound provided herein is administered (e.g., to an individual in need thereof) subcutaneously.

In some embodiments of the methods, a therapeutically effective amount of any compound or pharmaceutical composition provided herein is administered daily, weekly, bi-weekly, or monthly (e.g., for a period of time, such as, for example, one week, one month, one year, or longer). In some embodiments, a therapeutically effective amount of any compound or pharmaceutical composition provided herein is administered once or twice weekly. In some embodiments, the therapeutically effective amount of any compound or pharmaceutical composition provided herein is administered in 1 to 800 independent doses. In certain embodiments, the therapeutically effective amount of the compound or pharmaceutical composition has a concentration of compound of at or between 0.01 mg/kg of patient body weight to 1 mg/kg of patient body weight.

In some embodiments, the bone anabolic agent is a PTH or a PTHrP (SEQ ID NO: 1) or a derivative or fragment thereof (e.g., having bone anabolic activity)). In some embodiments, the bone anabolic agent is a PTH or a derivative or fragment thereof. In some embodiments, the bone anabolic agent is a PTHrP (SEQ ID NO: 1) or a derivative or fragment thereof. In some embodiments, the bone anabolic agent is a (e.g., synthetically) modified PTH or a derivative or fragment thereof. In some embodiments, the bone anabolic agent is a (e.g., synthetically) modified PTHrP (SEQ ID NO: 1) or a derivative or fragment thereof. In some embodiments, the bone anabolic agent is abaloparatide or a derivative or fragment thereof (e.g., having bone anabolic activity). In some embodiments, the bone anabolic agent is abaloparatide (SEQ ID NO: 2). In some embodiments, the bone anabolic agent is a (e.g., synthetically) modified abaloparatide.

In some embodiments, Z is a linear chain of amino acid residues. In some embodiments, Z is an AOP (e.g., comprising at least 4 glutamic acid amino acid residues or 4 aspartic acid amino acid residues).

In some embodiments, Z comprises at least 4 amino acid residues (e.g., 4 or more, 10 or more, 20 or more, 30 or more, 50 or more, 75 or more, or 100 or more). In some embodiments, Z comprises at most 100 amino acid residues (e.g., 100 or less, 75 or less, 50 or less, 30 or less, 20 or less, 10 or less, or 4 or less). In some embodiments, Z comprises not less than 4 and not more than 35 amino acids. In some embodiments, Z comprises not less than 4 and not more than 20 amino acids. In some embodiments, Z comprises not less than 6 and not more than 30 amino acids. In some embodiments, Z comprises not less than 8 and not more than 30 amino acids. In some embodiments, Z comprises not less than 8 and not more than 20 amino acids.

In some embodiments, the AOP the comprises from about 4 to about 20 amino acid residues (such as 4 to about 20 or about 4 to 20) or more amino acid residues, such as 4, 5, 6, 7, 8, 9, 10, 11, 12, 13, 14, 15, 16, 17, 18, 19 or 20. In various embodiments, the AOP comprises about 20 amino acid residues, such as 20 amino acid residues.

In some embodiments, the amino acids is aspartic acid (represented by the letter D), glutamic acid (represented by the letter E), or a mixture thereof. The amino acid residues can have D chirality, L chirality, or a mixture thereof. In some embodiments, the amino acid residue has D chirality. In some embodiments, the amino acid residue has L chirality. In some embodiments, Z comprises at least 4 (e.g., acidic) amino acid residues (e.g., having the same chirality (e.g., D- or L-amino acid residues)). In some embodiments, each of the at least 4 (e.g., acidic) amino acid residue has D chirality. In some embodiments, the aspartic acid is D-aspartic acid or L-aspartic acid. In some embodiments, the glutamic acid is D-glutamic acid or L-glutamic acid. In some embodiments, Z comprises not less than 4 and not more than 20 D-glutamic acid residues or L-glutamic acid residues. In some embodiments, Z comprises not less than 4 and not more than 20 D-aspartic acid residues or L-aspartic acid residues.

In some embodiments, Z comprises at least 4 (e.g., D-) glutamic acid amino acid residues (e.g., 4 to 20 D-glutamic acid amino acid residues) and/or at least 4 (e.g., D-) aspartic acid amino acid residues (e.g., 4 to 20 D-aspartic acid amino acid residues).

In some embodiments, Z comprises a mixture of (e.g., D-) glutamic acid amino acid residues and (e.g., D-) aspartic acid amino acid residues.

In some embodiments, Z comprises at least 4 repeating D-glutamic acid amino acid residues (e.g., DE4 or more, DE6 or more, DE8 or more, DE10 or more, DE15 or more, or DE20 or more, DE25 or more, DE30 or more, or DE35 or more). In some embodiments, Z comprises at least 10 repeating D-glutamic acid amino acid residues (e.g., DE4 or more, DE6 or more, DE8 or more, DE10 or more, DE15 or more, or DE20 or more, DE25 or more, DE30 or more, or DE35 or more). In some embodiments, X is abaloparatide or a derivative or fragment thereof (e.g., having bone anabolic activity) and Z is DE20.

In some embodiments, Y is a non-releasable linker. In some embodiments, Y is a non-releasable linker containing at least one carbon-carbon bond. In some embodiments, Y is a non-releasable linker containing at least one amide bond. In some embodiments, Y is a non-releasable linker containing at least one carbon-carbon bond and at least one amide bond.

In some embodiments, Y is a releasable linker. In some embodiments, Y is a releasable linker containing at least one disulfide (S—S). In some embodiments, Y is a releasable linker containing at least one ester (e.g., O(C═O)). In some embodiments, Y is a releasable linker containing at least one (e.g., protease-specific) amide bond.

In some embodiments, Y is a linker described elsewhere herein (e.g., hereinabove).

In some embodiments, Z is an osteotropic ligand described elsewhere herein (e.g., hereinabove).

In some embodiments, X is abaloparatide or a derivative or fragment thereof (e.g., having bone anabolic activity), Y is a non-releasable oligopeptide linker, and Z is DE20. In some embodiments, the compound is SEQ ID NO: 11.

In some embodiments, X is abaloparatide or a derivative or fragment thereof (e.g., having bone anabolic activity), Y is a releasable oligopeptide linker comprising at least one protease-specific amide bond, and Z is DE20.

In some embodiments, the compound is an imaging agent (e.g., a dye).

In some embodiments, the compound is a single-photon emission computer tomography/computed tomography (SPEC/CT) imaging agent.

In some embodiments, the compound is described elsewhere herein (e.g., hereinabove).

In some embodiments, the compound is SEQ ID NO: 3. In some embodiments, the compound is SEQ ID NO: 4.

In some embodiments, provided herein is a pharmaceutical composition comprising any compound provided herein (e.g., a compound having a structure of Formula (I), SEQ ID NO: 3 or SEQ ID NO: 4), or a pharmaceutically acceptable salt thereof (e.g., and at least one pharmaceutically acceptable carrier or excipient).

Still further provided in some embodiments herein is a method of treating a bone fracture. In some embodiments, the method comprises administering (e.g., subcutaneously) to a patient with a bone fracture an effective amount of a conjugate of formula X—Y—Z or a pharmaceutical composition comprising same, whereupon the bone fracture in the patient is treated. The patient can have diabetes mellitus, osteoporosis, or a maxillofacial fracture, such as a mandibular osteotomy stabilized with a microplate. The effective amount of the conjugate or the effective amount of the pharmaceutical composition can be administered by injection, such as subcutaneous injection.

Further embodiments and the full scope of applicability of the present disclosure will become apparent from the Detailed Description. However, it should be understood that the Detailed Description and specific examples are given by way of illustration only. Various changes and modifications within the spirit and scope of the present disclosure will become apparent to those skilled in the art.

### BRIEF DESCRIPTION OF THE FIGURES

FIG. 1A shows SEQ ID NO: 1, SEQ ID NO: 2, SEQ ID NO: 3, SEQ ID NO: 4, SEQ ID NO: 5, SEQ ID NO: 6 (the structure for a targeted conjugate of dasatinib), and SEQ ID NO: 7.

FIG. 1B shows SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

FIG. 2A shows chemical structures of a tetracycline, a mono-bisphosphonate, a polyphosphate, and an acidic oligopeptide conjugated to a radiolabeled tyrosyl cysteine.

FIG. 2B shows a graph of conjugate vs. fractured/healthy femurs ratio for bone-targeting ligands delivering ¹²⁵I tyrosyl cysteine payloads.

FIG. 3A shows a graph of tissue vs. percent injected dose/g of targeted SEQ ID NO: 1 compared to untargeted/free SEQ ID NO: 1.

FIG. 3B shows a graph of conjugate vs. percent injected dose/g of targeted SEQ ID NO: 1 and free SEQ ID NO: 1 in the fracture femurs of a mammal 24 hours post-injection.

FIG. 3C shows a graph of conjugate vs. fractured/healthy femurs ratio of targeted with SEQ ID NO: 1 as compared to untargeted/free SEQ ID NO: 1.

FIG. 4 shows a graph of tissue vs. percent injected dose/g of six different radio-iodinated payloads coupled to (L)Asp₁₀ 24 hours post-injection.

FIG. 5 shows a graph of tissue vs. percent injected dose/g of radio-iodinated casein kinase 2.3 peptide (CK2.3) coupled to 10 (L) aspartic acids relative to untargeted CK2.3.

FIG. 6 shows a graph of tissue vs. percent injected dose/gram of radio-iodinated CK2.3 coupled to 10 (L) aspartic acids, 10 (L) glutamic acids, or 10 (L) aminoadipic acids relative to untargeted CK2.3.

FIG. 7 shows a graph of tissue vs. percent injected dose/g of radio-iodinated CK2.3 coupled to 10 or 20 (L) glutamic acids relative to untargeted CK2.3.

FIG. 8 shows a graph of tissue vs. percent injected dose/g of radio-iodinated CK2.3 coupled to 20 L- or D-glutamic acids relative to untargeted CK2.3.

FIG. 9 shows a graph of hours post-injection vs. μg dye/mg tissue of 50456 (near-infrared (IR) fluorophore) coupled to 10 L- or D-aspartic acids in a mammal bearing midshaft femur fractures 10 days post-fracture at different time points post-injection.

FIG. 10 shows a graph of targeting ligands vs. percent injected dose/gram of radio-iodinated CK2.3 coupled to different acidic oligopeptides relative to untargeted CK2.3.

FIG. 11A shows a single-photon emission computer tomography/computed tomography (SPEC/CT) image of the Tc chelator EC20 chelating 99Tc linked to (D)Glu₁₀ acid.

FIG. 11B shows a SPEC/CT image of the Tc chelator EC20 chelating 99Tc linked to (D)Glu₂₀ acid.

FIG. 11C shows a graph of tissue vs. percent injected dose/g of the labeled (D)Glu₁₀ and (D)Glu₂₀ compounds in the different tissues.

FIG. 11D shows the structure of EC20(D)Glu₁₀ chelating 99Tc.

FIG. 12 shows the structure of a tri-bisphosphonate targeting ligand.

FIG. 13 shows a graph of agent tested vs. bone volume (BV) of targeted anabolic conjugates on a mammal after four weeks.

FIG. 14 shows a graph of agent tested vs. bone volume/total volume (BV/TV) of targeted anabolic conjugates on a mammal after four weeks.

FIG. 15 shows a graph of agent tested vs. maximum (max) load (N) of targeted anabolic conjugates on a mammal after four weeks.

FIG. 16 shows a graph of agent tested vs. work to fracture (mJ) of targeted anabolic conjugates on a mammal after four weeks.

FIG. 17 shows a graph of agent tested vs. post-yield displacement (mm) of targeted anabolic conjugates on a mammal after four weeks.

FIG. 18 shows a graph of days vs. blood sugar (mg/dl) of a type I diabetic rodent during the four-week treatment period using a compound provided herein.

FIG. 19 shows a graph of days vs. average % change in body mass for a type I diabetic fracture mice treated groups throughout treatment.

FIG. 20A shows a graph of agent tested vs. bone volume of targeted anabolic conjugates on a mammal after four weeks.

FIG. 20B shows a graph of agent tested vs. bone volume/total volume of targeted anabolic conjugates on a mammal after four weeks.

FIG. 21A shows a graph of agent tested with insulin vs. trabecular thickness of targeted anabolic conjugates on a mammal after four weeks.

FIG. 21B shows a graph of agent tested with insulin vs. trabecular spacing of targeted anabolic conjugates on a mammal after four weeks.

FIG. 22A shows a graph of agent tested with insulin vs. maximum force (N) of targeted anabolic conjugate on a mammal after four weeks.

FIG. 22B shows a graph of agent tested with insulin vs. work to fracture (mJ) of targeted anabolic conjugate on a mammal after four weeks.

FIG. 22C shows a graph of agent tested with insulin vs. modulus (MPa) of targeted anabolic conjugate on a mammal after four weeks.

FIG. 23A shows a graph of agent tested vs. BV of targeted anabolic conjugate on a mammal after four weeks.

FIG. 23B shows a graph of agent tested vs. bone volume/total volume (BV/TV) of targeted anabolic conjugate on a mammal after four weeks.

FIG. 24A shows a graph of agent tested vs. max load (N) of targeted anabolic conjugate on a mammal after four weeks.

FIG. 24B shows a graph of agent tested vs. work to fracture (mJ) of targeted anabolic conjugate on a mammal after four weeks.

FIG. 24C shows a graph of agent tested vs. stiffness (MPa) of targeted anabolic conjugate on a mammal after four weeks.

FIG. 25 shows a graph of agent tested vs. serum calcium concentration (mg/dl) of treatment on serum calcium in a mammal with a midshaft femur fracture model.

FIG. 26 shows a graph of days vs. distance traveled (cm) in locomotor open-field boxes for different treatment groups.

FIG. 27 shows a graph of days vs. time spent moving in locomotor open-field boxes for different treatment groups.

FIG. 28 shows a graph of days vs. mean velocity (cm/s) in locomotor open-field boxes for different treatment groups.

FIG. 29 shows graphs of agent vs. non-calcified area (mm 2) for defect and graft and cranial defect, agent vs. percent migrated (%) for screw, agent vs. gap diameter (mm) for mandibular osteotomy, and agent vs. max load (N) for mandibular osteotomy.

FIG. 30 shows a graph of hours vs. percent injected dose in blood (cpm/g) of compounds provided herein.

FIG. 31 shows a graph of hours vs. percent injected dose in bone (cpm/g) of compounds provided herein in fractured femurs and contralateral femurs.

FIG. 32 shows a graph of treatment of a compound provided herein vs. max load (N).

FIG. 33 shows a graph of treatment of a compound provided herein vs. work to fracture (mJ).

FIG. 34 shows a graph of treatment of a compound provided herein vs. max load (N).

FIG. 35 shows CT images of bone imaged three weeks after initiation of treatment with non-targeted abaloparatide.

### DETAILED DESCRIPTION

The present disclosure relates to the preparation and use of compounds and compositions that treat bone fractures. In some embodiments, the compounds, compositions, and methods leverage strategies to (e.g., selectively) localize the therapeutic agents to a bone fracture or other bone injury of interest. For example, the compounds, compositions, and methods provided may comprise an osteotropic ligand. In some embodiments, the compounds and compositions are formulated to exhibit increased retention time (such as due to increased resistance to degradation, for example) such that the frequency at which the compound or composition is readministered to maintain a therapeutically effective concentration at the targeted site (e.g., a fracture site) is reduced. The compounds, compositions, and methods hereof allow for significant advantages over conventional therapies used to treat bone fractures. For example, targeted therapies allow for a noninvasive way to maintain longer duration of therapeutic concentrations of drug relative to the traditional bolus administration used in local application of a therapeutic like in bone morphogenetic protein-2 (BMP2). This can result in more robust stimulation of healing and faster repair. The noninvasive nature can further allow physicians to control when and how long a drug is administered such that they can affect different phases of fracture healing and adjust treatment strategies to meet variability in patient healing times. It also can reduce systemic exposure and side effects and can avoid leakage in to neighboring tissue like the local application of anabolics.

Bone fractures can present in patients with osteoporosis. Osteoporosis can be a co-morbidity that individuals have later in life (e.g., >65 years old) and can be the result of a misbalance between the osteoblasts and the osteoclasts. In women, the misbalance can be triggered by the loss of estrogen during menopause. The loss in bone density due to the misbalance can lead to fragile bones that break with a relatively reduced force (compared to earlier in life). The misbalance in the bone basic unit can also slow the healing of fractures.

At least one in three women and one in five men over the age of 50 will suffer an osteoporotic fracture. Osteoporotic patients are at least twice as likely to get a fracture (e.g., due to sarcopenia and weakened bone) and, as such, osteoporotic fractures can be a challenge to the at-risk population. This population would benefit from a noninvasive strategy to accelerate bone fracture healing.

Similarly, diabetic patients have six times as many fractures as patients without diabetes. Diabetic patients may have twice as many nonunion fractures as healthy patients. The increase in fractures, in some instances, occurs from microstructural changes (e.g., in the extracellular matrix of the bone). Hyperglycemia can lead to non-enzymatic crosslinks between collagen strands. In addition, in type I diabetes mellitus, the loss of insulin production from the beta cells in the islets of Langerhans can lead to a reduction in bone mineral density (e.g., because insulin is anabolic for osteoblasts). Moreover, bone healing can be impaired from poor vascularization and neuropathy. Accelerating fracture repair in these patients may be important, since, for example, they may be more prone to co-morbidities when immobilized. Diabetic patients can be challenging orthopedic patients to treat with current bone healing treatment options.

While long bone fractures can require temporary immobilization and short-term, lifestyle changes, maxillofacial bone fractures can consistently result in severe decrements in quality of life (e.g., that often persists until the damaged bones are substantially repaired). This decline in quality of life can be from pain arising from the high density of nerve endings in craniofacial regions, the concentration of all five major senses in these areas, and the loss of crucial functions of this area, such as, for example, communication and mastication.

Although the physical and financial burdens of fractures in the United States have been frequently lamented, methods for treating these fractures have surprisingly not improved. Treatment methods, in some instances, rely on stabilization with rods, plates, and/or casts.

In many instances, osteogenic drugs approved to date are topically applied during surgery. For example, because surgery is not indicated for most fractures, the opportunity to employ these pharmacologic agents can be difficult.

In many instances, the metabolic turnover of approved bone anabolic agents is relatively fast which can restrict the duration of their therapeutic benefits to a brief window following topical application.

Additionally, leakage of locally applied anabolic drugs into surrounding tissues can lead to undesirable side effects including, for example, ectopic bone growth. In some instances, systemic administration of osteogenic agents stimulates unwanted anabolic processes in healthy tissues, such as, for example, nerves, muscles, and the vasculature. In some instances, hypercalcemia, hypertension, immunosuppression, and even cancer are concerns surrounding systemic administration of bone anabolic drugs.

A possible solution is bone targeting. To date, bone targeting has primarily focused on delivering payloads to orthopedic pathologies not related to fractures, such as osteoporosis, osteomyelitis, and bone metastases. Most of these treatments are bisphosphonates to deliver compounds selectively to bone. However, when treating bone fractures, it is imperative to deliver compounds selectively to the fracture site to avoid ectopic ossification that can occur when a drug is delivered nonspecifically to all bone. While tetracycline may be moderately selective for fractured over healthy bone, tetracyclines can be toxic to bone, liver and kidney and are thus not an ideal solution.

Several limitations also exist with using bisphosphonates for fracture targeting, including that they inhibit osteoclasts, which are essential for both normal skeletal remodeling and resolving of fracture calluses from woven bone into laminar bone. Another problem with using bisphosphonates as targeting ligands is that they have half-lives of up to 20 years in bone, which, depending on the stability of their therapeutic cargoes, can potentially lead to an undesirably prolonged stimulation of their molecular targets.

Similar targeting can be observed with ranelates. These compounds can be used as targeting molecules for many bone diseases and can be attached to anabolic agents to speed bone growth and healing. However, like bisphosphonates, they have a long bone half-life.

Given the issues with bisphosphonates, ranelates, and polyphosphates, including cumbersome synthesis and poor solubility, there remains a need for an osteotropic ligand that doesn't present such disadvantages. Desirably, the osteotropic ligand can deliver an attached peptidic, therapeutic agent to a fracture, in particular a fracture callus.

Abaloparatide (SEQ ID NO: 2) is an anabolic, 34-amino acid, synthetic analog of parathyroid hormone-related protein (PTHrP) (SEQ ID NO: 1). It can help promote bone growth and conserve bone density and can be used to treat osteoporosis. Abaloparatide (SEQ ID NO: 2) acts similarly to PTHrP (SEQ ID NO: 1) and targets, binds to, and activates the parathyroid hormone 1 (PTH1) receptor (PTH1R).

PTH1R is a G protein-coupled receptor (GPCR) expressed in osteoblasts and bone stromal cells. PTH1R, in turn, activates the cyclic adenosine monophosphate (cAMP) signaling pathway and the bone anabolic signaling pathway, leading to bone growth and increased bone mineral density and volume. The increase in bone mass and strength helps prevent/treat osteoporosis and decrease the risk of fractures.

Management of broken bones may be improved by continuously applying bone anabolic agents to a fracture over the entire course of the healing process. In some instances, hydroxyapatite is exposed on a broken bone. Molecules that bind with high affinity and specificity for hydroxyapatite, in some instances, may provide a treatment for targeting a bone anabolic agent to a fracture (e.g., and provide for continuous stimulation of fracture healing).

Patients with fractures can suffer a loss of function, for example, due to pain and lack of stability of a fracture. Conventional treatments for skeletal loss of function include, for example, improved stability by surgically implanting plates and rods, pain relief with nonsteroidal anti-inflammatory drugs (NSAIDS) and opioids, and locally applied anabolics. Surgical implantation of rods and plates is invasive and can be painful. For example, in some instance, patients use the fractured part of their body too quickly and thus delay healing. Opioids, can, in some instances, elicit cognitive impairment and are, for example, 1) the most commonly abused drug class (e.g., after orthopedic trauma in both young and aged populations) and 2) in some instances, responsible for the continuation of some pain syndromes following healing of the injury. Additionally, in some instances, opioids can induce dizziness and vertigo, which can, for example, result in falls that can further exacerbate existing bone injuries or cause new bone injuries. In some instances, NSAID use for fracture pain is discouraged as it can compromise the healing process. For example, administration of NSAIDs to alleviate pain may result in reduced bone density, decreased cartilage formation during early fracture fixation and, ultimately, nonunion of the bone defect. Mechanisms for this compromised healing may include, for example, delays in differentiation of stem cells and diminished BMP2 production. In some instances, patients continue to feel pain after treatment, resulting in a loss of function despite better radiographic outcomes. BMP2 is an approved therapy for treating bone fractures that can improve fracture healing, but has also been reported to, in some instances, increase pain after surgery, which may delay the gain of function following a fracture.

Compounds

Provided in some embodiments herein are compounds comprising acidic oligopeptides (AOPs) (e.g., 10-mers and 20-mers of acidic amino acids, 10-mers and 20-mers of either aspartic acid or glutamic acid, or various combinations of the foregoing). In some embodiments, AOPs effectively target spinal fusions. In some embodiments, 20-mers are more effective than 10-mers. In some embodiments, AOPs are highly selective compared to bisphosphonates and tetracyclines. In some embodiments, glutamic acid polymers and aspartic acid polymers have similar retention times at the delivery site. In some embodiments, while oligo-aspartic acids have reduced nonspecific retention in the kidneys, the slight increase in retention time observed with oligo-glutamic acid is transient. In some embodiments, both aspartic acid oligopeptides and glutamic acid oligopeptides (e.g., nearly quantitatively) clear from the kidneys after 18 hours. In some embodiments, AOPs target peptides of all chemical classes (e.g., hydrophobic, neutral, cationic, anionic, short oligopeptides, and long polypeptides). In some embodiments, this targeting is particularly beneficial as it allows for the development and broad use of this platform to develop other targeted therapeutics (e.g., many bone anabolic agents are peptidic, but their physical properties can vary greatly).

Provided in some embodiments herein are compounds comprising non-natural D enantiomers of AOPs, which can, in some instances, exhibit increased retention time on the fracture surface compared to the respective L enantiomers. This can be due to an increased resistance to degradation as compared to other compounds, for example. In some embodiments, increased retention time impacts the frequency that a therapeutic agent requires re-administration to maintain a therapeutically effective concentration at the targeted site of surgery (e.g., bone fracture). In some embodiments, increased retention time impacts the amount of a therapeutic agent required to be administered to elicit a targeted response (e.g., a therapeutic response). In some embodiments, linear AOPs are superior to branched AOPs (e.g., due to a reduction in, or the absence of, interference).

In some embodiments, targeted delivery of anabolic agents provides localization of therapeutic agents to bone fracture (e.g., via injection, such as, for example, subcutaneous injection, for example, at a distal site). In some embodiments, a compound provided herein is administered repeatedly to a patient (e.g., in need thereof). In some embodiments, a compound provided herein is administered at a relatively low dose to a patient (e.g., in need thereof). In some embodiments, a compound provided herein is administered at a safe dose to a patient (e.g., in need thereof). In some embodiments, a compound provided herein is administered at a therapeutic dose to a patient (e.g., in need thereof). In some embodiments, targeted delivery minimizes (e.g., if not eliminates) drift of an anabolic agent (e.g., into other tissues and unwanted mineralization). In some embodiments, bone growth in the region is stimulated for a relatively long time (e.g., to achieve relatively fast results (e.g., so that patients can regain their post-surgery mobility more quickly as compared to non-targeted delivery approaches)).

In some embodiments, provided herein is a compound having a structure of Formula (I):

X—Y—Z

or a pharmaceutically acceptable salt thereof, wherein:
X is a bone anabolic agent;
Y, when present, is a linker, which can be either releasable or non-releasable; and
Z is an osteotropic ligand.

Z can be any suitable osteotropic ligand. In some embodiments, the osteotropic ligand has an affinity for bone, e.g., hydroxyapatite. In some embodiments, the osteotropic ligand helps direct the compound (or a derivative or fragment thereof) to (e.g., healing) bone. In some embodiments, the osteotropic ligand has the potential to target the bone anabolic agent to a bone fracture or other bone injury. In some embodiments, the osteotropic ligand is a ligand with affinity for hydroxyapatite. In some embodiments, the osteotropic ligand is a ranelate, a bisphosphonate (e.g., alendronate), a tetracycline, a polyphosphate, an acidic molecule (such as a molecule with two or more carboxylic acids), a calcium chelator, a metal chelator, or an AOP. In some embodiments, the osteotropic ligand is an AOP. In some embodiments, the osteotropic ligand is a bisphosphonate selected from the group consisting of monobisphosphonate, tribisphosphonate, and polybisphosphonate.

In some embodiments, Z comprises at least 4 amino acid residues (e.g., 4 or more, 10 or more, 20 or more, 30 or more, 50 or more, 75 or more, or 100 or more). In some embodiments, Z comprises at most 100 amino acid residues (e.g., 100 or less, 75 or less, 50 or less, 30 or less, 20 or less, 10 or less, or 4 or less). In some embodiments, Z comprises not less than 4 and not more than 30 amino acids. In some embodiments, Z comprises not less than 4 and not more than 20 amino acids. In some embodiments, the AOP the comprises from about 4 to about 20 amino acid residues (such as 4 to about 20 or about 4 to 20) or more amino acid residues, such as 4, 5, 6, 7, 8, 9, 10, 11, 12, 13, 14, 15, 16, 17, 18, 19 or 20. In various embodiments, the AOP comprises about 20 amino acid residues, such as 20 amino acid residues. In other embodiments, the AOP can comprise more than 20 amino acid residues, such as 21, 22, 23, 24, 25, 30, 35, 40, 45, 50, 55, 60, 65, 70, 75, 80, 85, 90, 95 or as many as 100 amino acid residues.

In some embodiments, the amino acids can be aspartic acid (represented by the letter D), glutamic acid (represented by the letter E), or a mixture thereof. The amino acid residues can have D chirality, L chirality, or a mixture thereof. In some embodiments, the amino acid residue has D chirality. In some embodiments, the amino acid residue has L chirality. In some embodiments, the aspartic acid is D-aspartic acid or L-aspartic acid. In some embodiments, the glutamic acid is D-glutamic acid or L-glutamic acid. In some embodiments, Z comprises not less than 4 and not more than 20 D-glutamic acid residues or L-glutamic acid residues. In some embodiments, Z comprises not less than 4 and not more than 20 D-aspartic acid residues or L-aspartic acid residues.

In some embodiments, the AOP comprises one or more neutral or basic amino acids (e.g., provided that the AOP functions effectively as an osteotropic ligand). In some embodiments, the AOP comprises one or more synthetic amino acids (e.g., which can be acidic, neutral or basic).

In some embodiments, the AOP is linear (a linear chain) or branched (a branched chain). A linear chain is used in various embodiments. In some embodiments, the AOP can be cyclized.

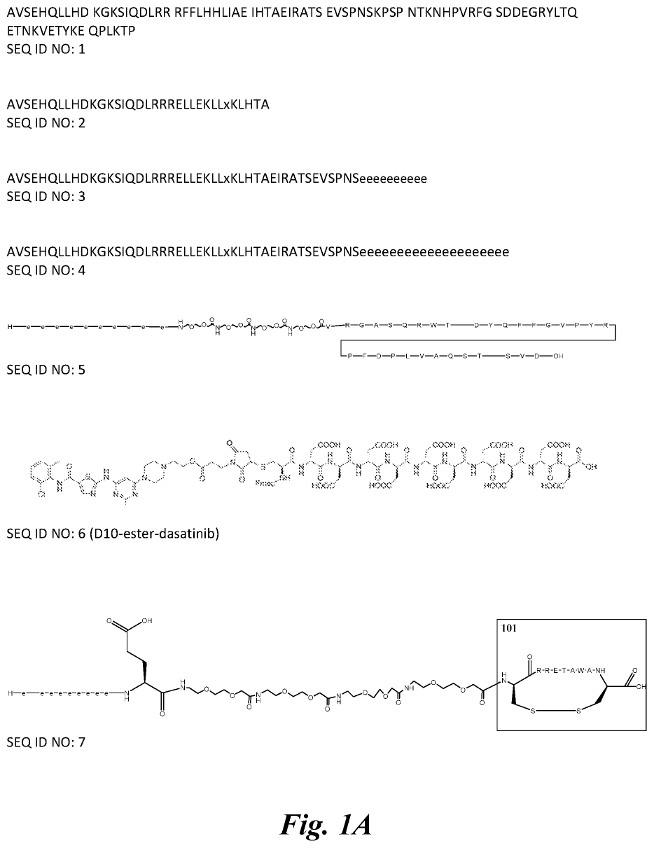

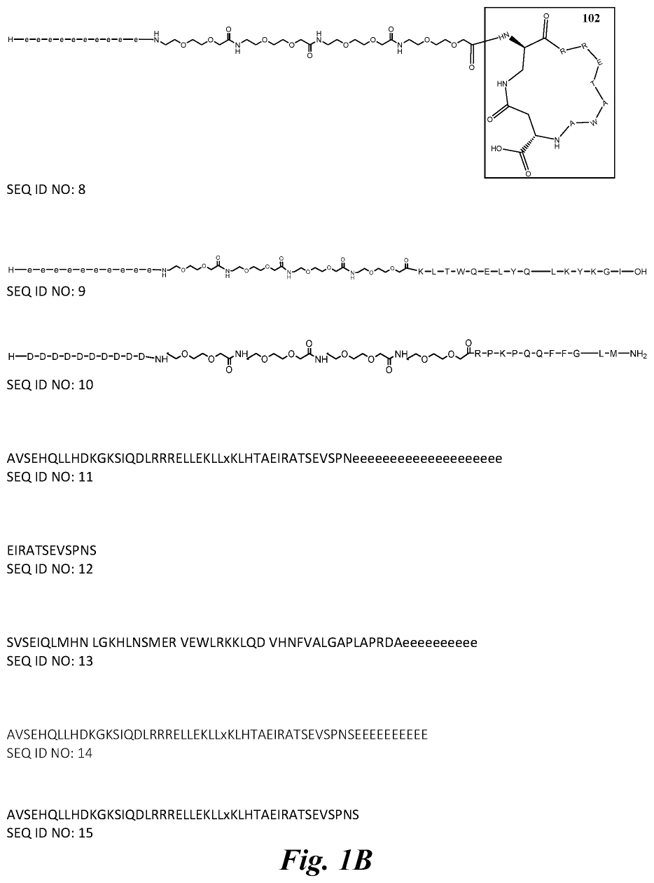

The osteotropic ligand (Z) can be a single unit, a polymer, a dendrimer, or multiple units. In some embodiments, the osteotropic ligand is a polymer. In some embodiments, the anabolic agent is cyclic. In some embodiments, the anabolic agent is a cyclic peptide. In some instances, a cyclic peptide is a compound (or radical thereof) consisting of two or more amino acids linked in a chain, wherein two portions of the compound combine to form a heterocyclic (e.g., peptide) molecule. Examples of cyclic peptides include, but are not limited to, structures 101 and 102 (see, e.g., FIGS. 1A and 1B).

In some embodiments, X is any suitable bone anabolic agent. In some embodiments, the bone anabolic agent is neutral, anionic, cationic, or hydrophobic. In some embodiments, the bone anabolic agent is an oligopeptide (e.g., comprising less than or equal to about 10 (or less than 10) amino acid residues, such as 10, 9, 8, 7, 6, 5 or 4 amino acid residues). In some embodiments, the bone anabolic agent comprises more than or equal to about 10 (or more than 10) amino acid residues, such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acid residues.

Examples of anabolic agents include, but are not limited to, abaloparatide (SEQ ID NO: 2), SEQ ID NO: 5 (e.g., a 34-residue peptide hormone that is secreted by the β-cells of the pancreatic islets; corresponds to Asp69-Leu102 of the E-peptide of proinsulin-like growth factor II (pro-IGF-II)), SEQ ID NO: 7 (e.g., ITGA conjugated with 10 glutamic acid residues (ITGA5)), SEQ ID NO: 6 (a conjugate with 10 glutamic acid residues), parathyroid hormone (PTH), parathyroid hormone related protein (PTHrP) (SEQ ID NO: 1), or a derivative of any of the foregoing (e.g., one or more amino acid mutations, such as insertions, deletions, and substitutions with a naturally occurring amino acid or a non-naturally occurring amino acid) having bone anabolic activity, or a fragment of any of the foregoing having bone anabolic activity.

In some embodiments, a bone anabolic agent followed by the designation D #, such as, e.g., D20, indicates that the bone anabolic agent is attached (or joined or connected, such as at an N-terminus or a C-terminus) to an osteotropic ligand (e.g., such as an orthotropic ligand having 20 aspartic acid residues). In some embodiments, a bone anabolic agent followed by the designation E #, e.g., E20, indicates that the bone anabolic agent is attached (or joined or connected, such as at an N-terminus or a C-terminus) to an osteotropic ligand (e.g., such as an orthotropic ligand having 20 glutamic acid residues).

In some embodiments, SEQ ID NO: 1 is AVSEHQLLHD KGKSIQDLRRRFFLHHLIAEIHTAEIRATSEVSPNSKPSPNTKNHPVRFGSDDEGRYLTQ ETNKVETYKE QPLKTP.

In some embodiments, SEQ ID NO: 2 is AVSEHQLLHDKGKSIQDLRRRELLEKLLxKLHTA, wherein x is α-aminoisobutyric acid (Aib). In some embodiments, the C-terminus of SEQ ID NO: 2 is amidated. In some embodiments, SEQ ID NO: 2 is AVSEHQLLHDKGKSIQDLRRRELLEKLLxKLHTA, wherein x is Aib, and the C-terminus is amidated.

In some embodiments, SEQ ID NO: 15 is AVSEHQLLHDKGKSIQDLRRRELLEKLLxKLHTAEIRATSEVSPNS, wherein x is Aib. In some embodiments, SEQ ID NO: 3 further comprises “eeeeeeeeee” where “e” signifies D-glutamic acid (e.g., SEQ ID NO: 3). In some embodiments, “EEEEEEEEEE” can be added to the end of SEQ ID NO: 15 to obtain SEQ ID NO: 14, where “E” indicates L-glutamic acid.

In some embodiments, SEQ ID NO: 4 is AVSEHQLLHDKGKSIQDLRRRELLEKLLxKLHTAEIRATSEVSPNSeeeeeeeeeeeeeeeeeeee, wherein x is Aib. In some embodiments, “e” signifies D-glutamic acid (e.g., “E” indicates L-glutamic acid, whereas “e” indicates D-glutamic acid). In some embodiments, SEQ ID NO: 4 is AVSEHQLLHDKGKSIQDLRRRELLEKLLxKLHTAEIRATSEVSPNSeeeeeeeeeeeeeeeeeeee, wherein x is Aib, and “e” signifies D-glutamic acid.

In some embodiments, the compound has at least 75% sequence identity or more, at least 85% sequence identity or more, at least 90% sequence identity or more, or at least 95% sequence identity or more to SEQ ID NO: 3. In some embodiments, the compound has at least 75% sequence identity or more, at least 85% sequence identity or more, at least 90% sequence identity or more, or at least 95% sequence identity or more to SEQ ID NO: 4. In some embodiments, the compound has at least 75% sequence identity or more, at least 85% sequence identity or more, at least 90% sequence identity or more, or at least 95% sequence identity or more to SEQ ID NO: 14.

In some embodiments of Formula (I), Y is a non-releasable linker. In some embodiments, Y is a non-releasable linker containing at least one carbon-carbon bond and/or at least one amide bond. In some embodiments, Y is a releasable linker. In some embodiments, Y is a releasable linker containing at least one disulfide (S—S), ester (e.g., —O(C═O)—), and/or protease-specific amide bond.

In some embodiments, the targeting molecule (i.e., osteotropic ligand) does not cleave from the drug/anabolic agent for the compound to be therapeutically effective in vivo. This can be advantageous as it can allow for the use of osteotropic ligands and compositions comprising anabolic agents because only a negligible amount (if any) of the anabolic agent is released (e.g., systemically) prior to the targeted delivery of the compound to the bone fracture site or other targeted site. In some embodiments, tuning the releasing properties of active components is a difficult aspect of the preparation of effective pharmaceutical compositions. In some embodiments, the compounds comprising the non-releasable linkers provided herein avoid the difficulties of the preparation of effective pharmaceutical compositions (e.g., by removing the necessity of timing the release). In some embodiments, the anabolic agent of the compound provided herein is active when bound (e.g., conjugated to the osteotropic ligand). Accordingly, in some embodiments, the compounds comprising targeting molecules (e.g., Z) conjugated with a non-releasable linker (e.g., Y) can reduce systemic exposure and/or systemic adverse effects of the anabolic agents (X) linked therewith.

In some embodiments, a conjugate comprising a non-releasable linker reduces or eliminates toxicity of a component released from the conjugate in its free form (e.g., a free form of a compound and/or ligand provided herein).

Both releasable and non-releasable linkers can be engineered to optimize biodistribution, bioavailability, and PK/PD (e.g., of the compound) and/or to increase uptake (e.g., of the compound) into the targeted tissue pursuant to methodologies commonly known in the art or hereinafter developed such as through PEGylation and the like. The linkers can further be engineered in view of the molecular target (e.g., whether the target is intracellular or extracellular) pursuant to concepts known in the art. In some embodiments, the linker is configured to avoid significant release of a pharmaceutically active amount of the anabolic agent in circulation prior to capture by a cell (e.g., a bone cell).

In some embodiments, linkers can comprise one or more spacers (e.g., to facilitate a particular release time, facilitate an increase in uptake into a targeted tissue, and/or optimize biodistribution, bioavailability, and/or PK/PD of a compound). A spacer can comprise one or more of alkyl chains, polyethylene glycols (PEGs), peptides, sugars, peptidoglycans, clickable linkers (e.g., triazoles), rigid linkers such as poly-prolines and poly-piperidines, and the like.

In some embodiments, the one or more linkers of the compounds provided herein can comprise PEG, a PEG derivative, or any other linker known in the art or hereinafter developed that can achieve the purpose set forth herein. In some embodiments, the linker is repeated n times, where n is a positive integer.

Conjugates can be synthesized in accordance with methods known in the art and exemplified herein, such as solid phase peptide synthesis.

Pharmaceutical Compositions

The compounds described herein can be administered alone or formulated as a pharmaceutical composition comprising the compound or compounds and one or more pharmaceutically acceptable excipients. As used herein, the terms “composition” generally refers to any product comprising more than one ingredient, including the compounds described herein. It is to be understood that the compositions described herein can be prepared from isolated compounds or from salts, solutions, hydrates, solvates, and other forms of the compounds. Certain functional groups, such as hydroxy, amino, and like groups, can form complexes with water and/or various solvents, in the various physical forms of the compounds. It is also to be understood that the compositions can be prepared from various amorphous, non-amorphous, partially crystalline, crystalline, and/or other morphological forms of the compounds, and the compositions can be prepared from various hydrates and/or solvates of the compounds. Accordingly, such pharmaceutical compositions that recite compounds include each of, or any combination of, or individual forms of, the various morphological forms and/or solvate or hydrate forms of the compounds.

One embodiment provides a pharmaceutical composition comprising a compound of Formula (I) or any compound covered by such formulae, or a pharmaceutically acceptable salt thereof, and at least one pharmaceutically acceptable excipient.

One embodiment provides a pharmaceutical composition comprising an effective amount of a therapeutically (or prophylactically) effective compound of Formula (I) or any compound covered by such formulae, or a pharmaceutically acceptable salt thereof, and at least one pharmaceutically acceptable excipient.

In some embodiments, provided herein is a pharmaceutical composition comprising a therapeutically effective amount of any compound provided herein that can be administered (e.g., subcutaneously) to a patient in need thereof. In various embodiments, the composition is an injectable composition, such as a composition that is suitable for subcutaneous injection.

Compounds and/or compositions described herein may be administered in unit dosage forms and/or compositions containing one or more pharmaceutically acceptable carriers, adjuvants, diluents, excipients, and/or vehicles, and combinations thereof.

As used herein, the term “administering” generally refers to any and all means of introducing compounds described herein to the host subject including, but not limited to, by oral, intravenous, intramuscular, subcutaneous, transdermal, inhalation, buccal, ocular, sublingual, vaginal, rectal, and like routes of administration.

Administration of the compounds as salts can be appropriate. Examples of acceptable salts include, without limitation, alkali metal (for example, sodium, potassium or lithium) or alkaline earth metals (for example, calcium) salts; however, any salt that is generally non-toxic and effective when administered to the subject being treated is acceptable. In at least one embodiment, the salt can be ammonium acetate salt. Similarly, “pharmaceutically acceptable salt” refers to those salts with counter ions which may be used in pharmaceuticals. Such salts may include, without limitation: (1) acid addition salts, which can be obtained by reaction of the free base of the parent compound with inorganic acids, such as hydrochloric acid, hydrobromic acid, nitric acid, phosphoric acid, sulfuric acid, perchloric acid, and the like, or with organic acids, such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid, malonic acid, and the like; or (2) salts formed when an acidic proton present in the parent compound either is replaced by a metal ion, e.g., an alkali metal ion, an alkaline earth ion, or an aluminum ion, or coordinates with an organic base, such as ethanolamine, diethanolamine, triethanolamine, trimethamine, N-methylglucamine, and the like. Pharmaceutically acceptable salts are well-known to those skilled in the art, and any such pharmaceutically acceptable salts are contemplated.

Acceptable salts can be obtained using standard procedures known in the art, including (without limitation) reacting a sufficiently acidic compound with a suitable base, affording a physiologically acceptable anion. Suitable acid addition salts are formed from acids that form non-toxic salts. Illustrative, albeit nonlimiting, examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Suitable base salts of the compounds described herein are formed from bases that form non-toxic salts. Illustrative, albeit nonlimiting, examples include the arginine, benzathine, calcium, choline, diethylamine, diolamine, glycine, lysine, magnesium, meglumine, olamine, potassium, sodium, tromethamine and zinc salts. Hemi-salts of acids and bases, such as hemisulphate and hemicalcium salts, also can be formed.

The compounds can be formulated as pharmaceutical compositions and administered to a mammalian host, such as a human patient, in a variety of forms adapted to the chosen route of administration. For example, the pharmaceutical composition can be formulated for and administered via intraosseous, intravenous, intraarterial, intraperitoneal, intracranial, intramuscular, topical, inhalation and/or subcutaneous routes. In at least one embodiment, a compound and/or composition can be administered directly (via injection, placement or otherwise) to a defect cavity in the impaired bone tissue and/or at a fracture site. In at least one embodiment, the compounds can be systemically administered in combination with a pharmaceutically acceptable vehicle, such as an inert diluent or an assimilable edible carrier. For oral therapeutic administration, the active compound can be combined with one or more excipients and used in the form of ingestible tablets, buccal tablets, troches, capsules, elixirs, suspensions, syrups, wafers, and the like. The percentage of the compositions and preparations can vary and may be between about 1 to about 99% weight of the active ingredient(s) and a binder, excipients, a disintegrating agent, a lubricant, and/or a sweetening agent (as are known in the art). The amount of active compound in such therapeutically useful compositions is such that an effective dosage level will be obtained.

The preparation of parenteral compounds/compositions under sterile conditions, for example, by lyophilization, can readily be accomplished using standard pharmaceutical techniques well-known to those skilled in the art. In at least one embodiment, the solubility of a compound used in the preparation of a parenteral composition can be increased by the use of appropriate formulation techniques, such as the incorporation of solubility-enhancing agents.

As previously noted, the compounds/compositions can also be administered via infusion or injection (e.g., using needle (including microneedle) injectors and/or needle-free injectors). Solutions of the active composition can be aqueous, optionally mixed with a nontoxic surfactant and/or contain carriers or excipients, such as salts, carbohydrates and buffering agents (preferably at a pH of from 3 to 9), but, for some applications, they may be more suitably formulated as a sterile non-aqueous solution or as a dried form to be used in conjunction with a suitable vehicle, such as sterile, pyrogen-free water or phosphate-buffered saline. For example, dispersions can be prepared in glycerol, liquid PEGs, triacetin, and mixtures thereof and in oils. Under ordinary conditions of storage and use, these preparations can further contain a preservative to prevent the growth of microorganisms.

The pharmaceutical dosage forms suitable for injection or infusion can include sterile aqueous solutions or dispersions or sterile powders comprising the active ingredients that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example and without limitation, water, ethanol, a polyol (e.g., glycerol, propylene glycol, liquid PEG(s), and the like), vegetable oils, nontoxic glyceryl esters, and/or suitable mixtures thereof. In at least one embodiment, the proper fluidity can be maintained by the formation of liposomes, by the maintenance of the required particle size in the case of dispersions or by the use of surfactants. The action of microorganisms can be prevented by the addition of various antibacterial and antifungal agents, such as parabens, chlorobutanol, phenol, sorbic acid, thimerosal, and the like. In certain cases, it can be desirable to include one or more isotonic agents, such as sugars, buffers, or sodium chloride. Prolonged absorption of the injectable compositions can be brought about by the incorporation of agents formulated to delay absorption, for example, aluminum monostearate and gelatin.

Sterile injectable or infusible solutions can be prepared by incorporating the active compound and/or composition in the required amount of the appropriate solvent with one or more of the other ingredients set forth above, as required, followed by filter sterilization. In the case of sterile powders for the preparation of sterile injectable solutions, the preferred methods of preparations are vacuum drying and freeze-drying, which yield a powder of the active ingredient plus any additional desired ingredient present in the previously sterile-filtered solutions.

For topical administration, it can be desirable to administer the compounds to the bone as compositions or formulations in combination with an acceptable carrier, which may be a solid, a liquid, or a gel matrix. For example, in certain embodiments, useful liquid carriers can comprise water, alcohols or glycols or water-alcohol/glycol blends, in which the compounds can be dissolved or dispersed at effective levels, optionally with the aid of non-toxic surfactants. Additionally or alternatively, adjuvants, such as antimicrobial agents, can be added to optimize the properties for a given use. The resultant liquid compositions can be applied from absorbent pads, used to impregnate bandages and/or other dressings, sprayed onto the targeted area using pump-type or aerosol sprayers, or simply applied directly to a desired area of the subject (e.g., a fracture site).

Thickeners, such as synthetic polymers, fatty acids, fatty acid salts and esters, fatty alcohols, modified celluloses or modified mineral materials can also be employed with liquid carriers to form spreadable pastes, gels, ointments, soaps, and the like for application directly to the skin of the subject.

As used herein, the term “therapeutically effective dose” means (unless specifically stated otherwise) a quantity of a compound which, when administered either one time or over the course of a treatment cycle affects the health, well-being or mortality of a subject (e.g., and without limitation, supports or promotes the healing of bone fractures or bone growth).

In some embodiments, the therapeutically effective amount of any compound or pharmaceutical composition provided herein is determined in accordance with methods known in the art (e.g., animal models, human data, and human data for compounds that exhibit similar pharmacological activities). Useful dosages of the compounds can be determined by comparing their in vitro activity and the in vivo activity in animal models. Methods of the extrapolation of effective dosages in mice and other animals to human subjects are known in the art. Indeed, the dosage of the compound can vary significantly depending on the condition of the host subject, the bone fracture being treated, the route of administration of the compound and tissue distribution, and the possibility of co-usage of other therapeutic treatments (for example, in conjunction with the administration of other injectable compositions for promoting bone growth such as growth factors, stem cells, natural grafts, biologic- and synthetic-based tissue-engineered scaffolds and the like, hardware implantation, and/or ultrasound therapies and the like; and/or in conjunction with the administration of other therapeutics such as, for example, insulin). In some embodiments, the therapeutically effective amount of any compound or pharmaceutical composition provided herein is determined by taking into consideration, for example, the potency of X of Formula (I) (e.g., the type of anabolic agent employed), body weight, mode of administration (e.g., subcutaneously), disease or condition being treated, disease or condition its severity, the like, or any combination thereof. The amount of the composition required for use in treatment (e.g., the therapeutically effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise.

In some embodiments, the therapeutically effective amount of any compound or pharmaceutical composition provided herein is from about 0.01 mg/kg/day up to about 1,000 mg/kg/day. For example, therapeutically effective amounts or doses can range from about 0.05 mg/kg of patient body weight to about 30.0 mg/kg of patient body weight, or from about 0.01 mg/kg of patient body weight to about 5.0 mg/kg of patient body weight, including, but not limited to, 0.01 mg/kg, 0.02 mg/kg, 0.03 mg/kg, 0.04 mg/kg, 0.05 mg/kg, 0.1 mg/kg, 0.2 mg/kg, 0.3 mg/kg, 0.4 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, and 5.0 mg/kg, all of which are kg of patient body weight. Intravenous doses can be several orders of magnitude lower. In some embodiments, the compound the therapeutically effective amount of any compound or pharmaceutical composition provided herein is administered (e.g., subcutaneously) daily, weekly, bi-weekly, monthly, or bi-monthly.

In some embodiments, the therapeutically effective amount of any compound or pharmaceutical composition provided herein is administered in 1 to 800 doses. In certain embodiments, the therapeutically effective amount of the compound or pharmaceutical composition has a concentration of compound of at or between 0.01 mg/kg of patient body weight to 1 mg/kg of patient body weight.

In some embodiments, the therapeutically effective amount (e.g., administered to the individual) of any compound or pharmaceutical composition provided herein (e.g., SEQ ID NO: 1, SEQ ID NO: 2, SEQ ID NO: 3, SEQ ID NO: 4, etc.) is from about 0.01 mg/kg/day up to about 1,000 mg/kg/day. In some embodiments, the therapeutically effective amount (e.g., administered to the individual) of any compound or pharmaceutical composition provided herein (e.g., SEQ ID NO: 1, SEQ ID NO: 2, SEQ ID NO: 3, SEQ ID NO: 4, etc.) is about 1 μg/dose to about 10 mg/dose. In some embodiments, the therapeutically effective amount (e.g., administered to the individual) of any compound or pharmaceutical composition provided herein (e.g., SEQ ID NO: 1, SEQ ID NO: 2, SEQ ID NO: 3, SEQ ID NO: 4, etc.) is about 50 μg/dose to about 5 mg/dose. In some embodiments, the therapeutically effective amount (e.g., administered to the individual) of any compound or pharmaceutical composition provided herein (e.g., SEQ ID NO: 1, SEQ ID NO: 2, SEQ ID NO: 3, SEQ ID NO: 4, etc.) is about 0.01 nmol/kg/dose to about 10 ng/kg/dose. In some embodiments, the therapeutically effective amount (e.g., administered to the individual) of any compound or pharmaceutical composition provided herein (e.g., SEQ ID NO: 1, SEQ ID NO: 2, SEQ ID NO: 3, SEQ ID NO: 4, etc.) is about 0.1 nmol/kg/dose to about 5 ng/kg/dose.

The total therapeutically effective amount of the compound can be administered in single or divided doses and can, at the practitioner's discretion, fall outside of the typical range given herein. In some embodiments, a therapeutically effective amount of any compound or pharmaceutical composition provided herein is administered once or twice weekly. In some embodiments, a therapeutically effective amount of any compound or pharmaceutical composition provided herein is administered once weekly. In some embodiments, a therapeutically effective amount of any compound or pharmaceutical composition provided herein is administered twice weekly.

The effective amount of an X—Y—Z conjugate, such as a pharmaceutical composition comprising an effective amount of the conjugate, can be administered by any suitable route. An example of a suitable route is by injection, such as subcutaneous injection. Other examples of suitable routes are parenterally and enterally.

Methods of Treatment

Provided in some embodiments herein are methods of treating a bone fracture (e.g., in an individual in need thereof) using the compounds and/or compositions provided. The compounds, compositions and methods can leverage strategies to (e.g., selectively) target the fracture site to prevent off-target effects of the anabolic agents present within the compounds or compositions.

In some embodiments, a method of treating a bone fracture in a patient (e.g., in need thereof) comprises administering (e.g., subcutaneously) to the patient a therapeutically effective amount of a compound (e.g., having a structure of Formula (I)) or a pharmaceutical composition provided herein, thereby treating the bone fracture in the patient. In some embodiments, administration of the therapeutically effective amount of the compound or pharmaceutical composition provided herein results in a reduction of pain in the patient within 2-3 weeks following administration. In some embodiments, administration of the therapeutically effective amount of the compound or pharmaceutical composition provided herein results in a reduction of pain in the patient within 3 weeks following administration. In some embodiments, the patient (e.g., in need thereof) is susceptible to a bone fracture. For example, the patient can have one or more comorbidities selected from the group consisting of mellitus, osteoporosis, a maxillofacial injury (e.g., a maxillofacial fracture), a maxillofacial deficiency, and a maxillofacial defect.

Methods for promoting bone growth in a patient (e.g., in need thereof) are also provided. In some embodiments, such methods comprise administering (e.g., subcutaneously) to the patient a therapeutically effective amount of a compound (e.g., having a structure of Formula (I)) or a pharmaceutical composition provided herein, thereby increasing a bone mineral density in a bone of the patient as compared to pre-treatment. In some embodiments, the increased bone mineral density in the bone occurs at a fracture site. In some embodiments, the increased bone mineral density in the bone occurs at one or more resorption pits (e.g., where the patient is experiencing osteoporosis) present in the bone prior to the administering step.

In some embodiments, the patient has diabetes mellitus. In some embodiments, the patient has diabetes mellitus and X is abaloparatide (SEQ ID NO: 2), SEQ ID NO: 7, SEQ ID NO: 8 (a conjugate with 10 glutamic acid residues), a derivative (e.g., one or more amino acid mutations, such as insertions, deletions, and substitutions with a naturally occurring amino acid or a non-naturally occurring amino acid) thereof having bone anabolic activity, or a fragment thereof having bone anabolic activity.

In some embodiments, the patient has osteoporosis. In some embodiments, the patient has osteoporosis and X of Formula (I) is a parathyroid hormone, a derivative (e.g., one or more amino acid mutations, such as insertions, deletions, and substitutions with a naturally occurring amino acid or a non-naturally occurring amino acid) thereof having bone anabolic activity, or a fragment thereof having bone anabolic activity.

In some embodiments, the patient has a maxillofacial injury, such as, for example, a microplate stabilized mandibular osteotomy (i.e. a maxillofacial fracture). The patient can have a defect filled with a bone graft (e.g., osseointegration), a prosthetic implant (e.g., plate, screw, and/or osseointegration), or a physician-induced bone defect (e.g., mandibular osteotomy, cranial defect, or a defect with graft). In some embodiments, the patient has a maxillofacial deficiency.

In some embodiments, the method can further comprise administering a second therapy to the patient for treating the bone fracture (e.g., pain medication, bone grafts, implants (e.g., mesh), growth hormone and the like) or one or more comorbidities of the patient. In some embodiments where the patient has at least diabetes mellitus, administering the second therapy can comprise administering a therapeutically effective amount of insulin to the patient. In some embodiments where the patient suffers from at least a bone fracture, administering the second therapy can comprise implantation of hardware (e.g., mesh or pins) or one or more therapeutic compounds at a bone fracture site.

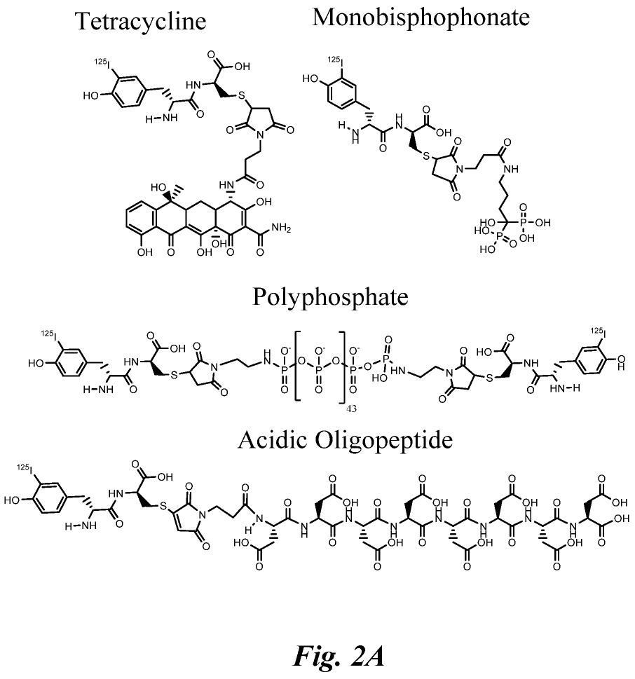

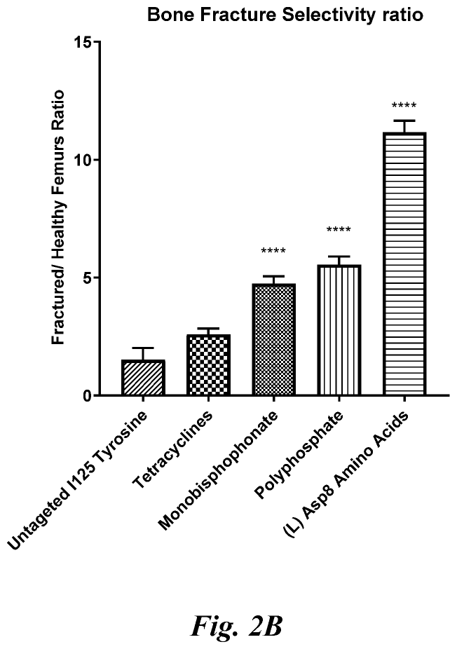

As shown in FIG. 2A, in some embodiments, when administered, the targeting ligands of certain compounds provided herein (e.g., where Z of Formula (I) comprises hydroxyapatite targeting ligands) can accumulate with different specificities at the femur fracture site (see FIG. 2B). In some embodiments, tetracycline, mono-bisphosphonate, polyphosphate and (L)Asp8 (an acidic oligopeptide consisting of eight L-aspartic acids) labeled with ¹²⁵I-tyrosine and injected intravenously into fracture-bearing mice accumulate with different specificities at the femur fracture site (FIG. 2B). In some embodiments, the selectivity ratio of ¹²⁵I-labeled tetracycline in the fractured to healthy femur is 2.6, which is significant because it supports the development of a drug that elicits its anabolic effect primarily at the fracture site instead of throughout the skeleton.

In some embodiments, the fractured-to-healthy ratio continuously increases as the tetracycline ligand (Z of Formula (I)) was exchanged for alendronate, polyphosphate, and/or an acidic octa-aspartic acid. In some instances, octa-aspartic acid has the highest specificity (e.g., of the ligands tested) for fractured over healthy bone with a selectivity ratio of 11.2. In some instances, tetracycline has the highest specificity (e.g., of the ligands tested for Z of Formula (I)) for fractured over healthy bone with a selectivity ratio.

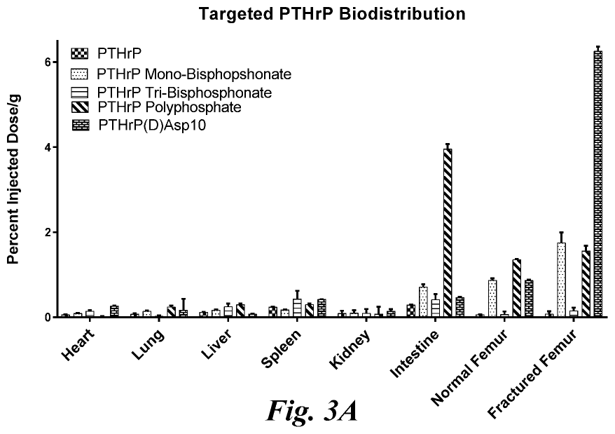

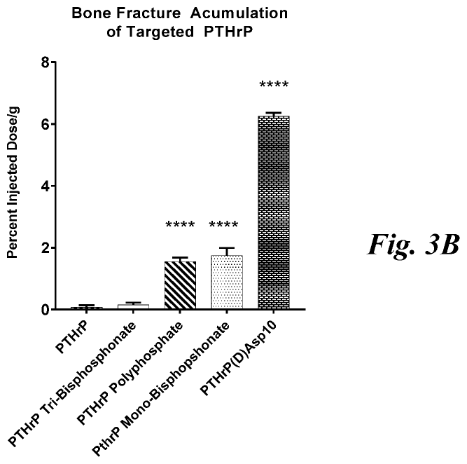

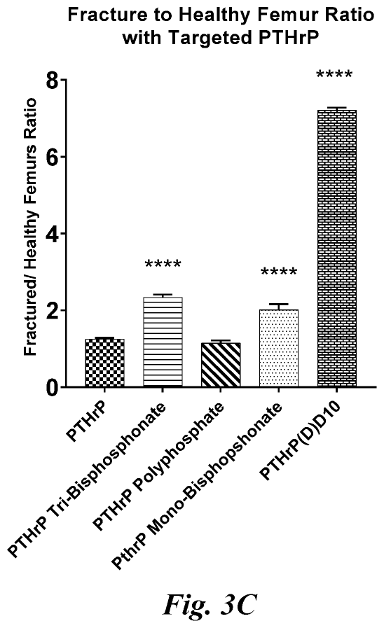

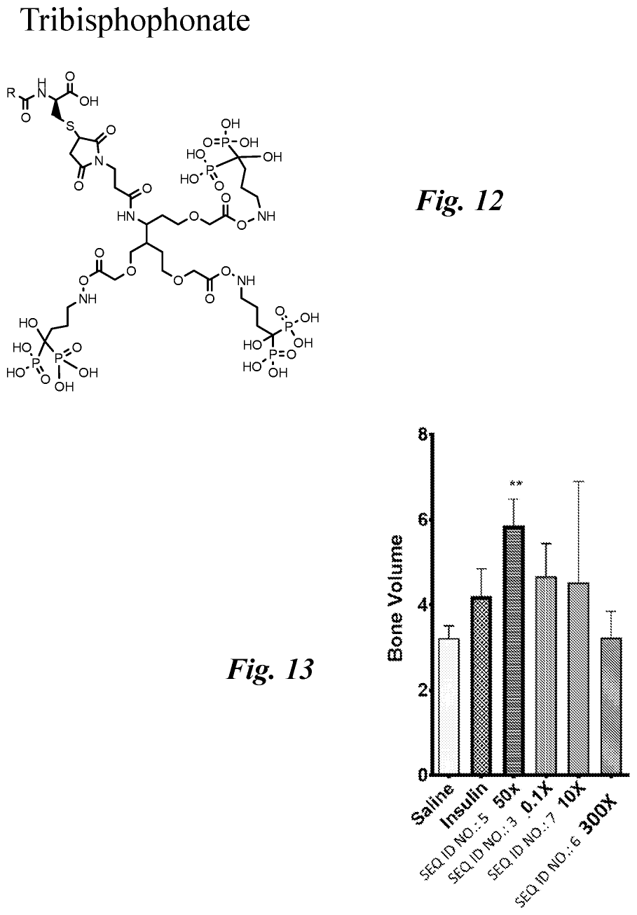

In some instances, mono-bisphosphonate and polyphosphate exhibit reduced specificity for fractured bone. In some instances, mono-bisphosphonate and polyphosphate peptide-targeting abilities are compared with more specific osteotropic ligands. In some instances, a N-terminal 34 amino acids of PTHrP (SEQ ID NO: 1) is labeled with ¹²⁵I and tethered to a mono-bisphosphonate (e.g., alendronate), a tri-bisphosphonate (e.g., comprising of three alendronates attached to a central hub (FIG. 12)), a polyphosphate (e.g., consisting of 45 phosphates connected by anhydride linkages), or a deca-aspartic acid (e.g., similar to the octa-aspartic acid described herein). For example, the biodistribution of targeted SEQ ID NO: 1 is shown in FIG. 3A, which is a graph of tissue vs. percent injected dose/g.

In some instances, the mono-bisphosphonate (e.g., alendronate) as the targeting ligand (e.g., Z of Formula (I)) enables delivery of an (e.g., moderate) amount of ¹²⁵I-SEQ ID NO: 1 (1-34) to the fracture site. In some instances, the mono-bisphosphonate (e.g., alendronate) as the targeting ligand (e.g., Z of Formula (I)) has a specificity of about 2:1 for broken bone over healthy bone (FIG. 3C). Provided in some instances herein, such as, for example FIG. 3C (e.g., a graph of conjugate vs. fractured/healthy femurs ratio), is the selectivity ratio of the fracture callus and the contralateral healthy femur targeted with a compound or composition described herein (e.g., SEQ ID NO: 1).

In some instances, provided herein is a compound (e.g., ligand) comprising one or more alendronate (e.g., at least one alendronate, at least two alendronate, at least three alendronate, or more). In some instances, a compound provided herein (e.g., comprising three alendronates) has diminished fracture targeting. In some instances, a compound provided herein (e.g., a polyphosphate) has minimal ¹²⁵I-SEQ ID NO: 1 (1-34) delivery to the fracture surface (e.g., with only 1.55% of injected drug being present on the fracture surface 24 hours later). In some instances, a compound provided herein (e.g., an AOP (e.g., comprised of 10 aspartic acids)) has a (e.g., high) specific delivery to the fractured bone (e.g., with 3.5 times more specificity for the fracture and accumulation in the fracture than mono-bisphosphonates (FIG. 3B). Provided in some instances herein, such as, for example FIG. 3B (e.g., a graph of conjugate vs. percent injected dose/g), is the bone fracture accumulation of targeted compound (e.g., SEQ ID NO: 1) and free compound (e.g., SEQ ID NO: 1) in the fracture (e.g., femurs of mice 24 hours post-injection).

In some instances, an AOP described herein delivers an attached anabolic peptide described herein (e.g., X of Formula (I)) to a fracture surface. In some instances, (1) chemical characteristics of payload, (2) AOP side chain structure, (3) AOP length, (4) AOP branching, and/or (5) AOP stability affects the ability of an AOP to deliver an attached anabolic peptide to a fracture surface.

In some instances, the characteristics of a therapeutic payload described herein (e.g., size, charge, and hydrophobicity) may affect an attached AOP to concentrate (e.g., the active drug; e.g., X of Formula (I)) at a fracture site. In some instances, CK2.3 (e.g., a cationic peptide with a net charge of +5), osteopontin-derived peptide (ODP) (e.g., an anionic peptide with a net charge of −3), or chemotactic cryptic peptide (CTC) (e.g., a neutral peptide with a net charge of 0) is the therapeutic payload. In some instances, P4 (e.g., a hydrophobic peptide with a hydrophobicity index (GRAVY) of 0.49) is the therapeutic payload. In some instances, F109 (e.g., having a chain length of 9 amino acids) or casein kinase 2.3 peptide (CK2.3) (e.g., having a chain length of 39 and 30 amino acids). In some instances, the therapeutic payload is provided in Table 1. In some instances, the bone anabolic peptide is attached to L-Asp₁₀, radiolabeled with iodogen ¹²⁵I (e.g., and injected into mice with fractured femurs and allowed to circulate for 18 hours before evaluation for tissue biodistribution).

TABLE 1

| | Representative | |
|---|---|---|
| Peptide Payload | Chemical Class | Sequence |
| F109C: heparin-binding | Short | YKRSRYTC [SEQ ID NO: 16] |
| domain of FGF2 | Oligopeptide | |
| PACAPC: pituitary | Long | HSDGIFTDSYSRYRKQMAVKKYLAAV |
| adenylate cyclase-activating | Polypeptide | LGKRYKQRVKNKC [SEQ ID NO: 17] |
| polypeptide | | |
| CTC-C: chemotactic | Neutral | YIAGVGGEKSGGFYC [SEQ ID NO: 18] |
| cryptic peptide (CTC), | | |
| derived from the CTX | | |
| region of collagen type III | | |
| Ck2.3C: casein kinase 2 beta | Cationic | RQIKIWFQNR RMKWKKIPVG |
| chain | | ESLKDLIDQC [SEQ ID NO: 19] |
| ODPC: osteopontin-derived | Anionic | DVDVPDGRGDSLAYGC [SEQ ID NO: |
| peptide | | 20] |
| P4C: BMP-2 fragment | Hydrophobic | KIPKASSVPTELSAISTLYLC [SEQ ID |
| | | NO: 21] |

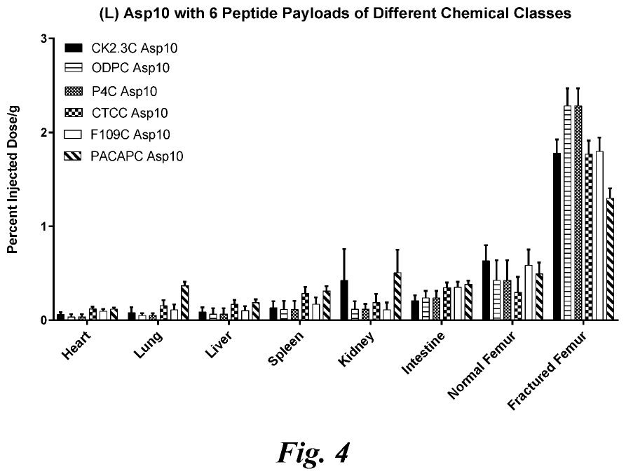

Provided herein is a graph of tissue vs. percent injected dose/g, which shows the biodistribution of six different radio-iodinated payloads coupled to (L)Asp₁₀ 24 hours post-injection (prepared in accordance with certain embodiments hereof) into ND-4 Swiss-Webster mice (n=6) bearing midshaft femur fractures 10 days post-fracture (e.g., FIG. 4). In some instances, the chemical properties of the peptides exerted little impact on the ability of L-Asp₁₀ to target them to fracture surfaces (see FIG. 4). In some instances, the 39-amino acid PACAP differed somewhat from the other anabolic peptides in fracture targetability. In some instances, neither payload size nor other major chemical/physical variables exerts a consistent impact on AOP-mediated bone targeting (e.g., because a peptide of similar length (CK2.3) displayed no reduction in fracture accumulation). In some instances, other anabolic cargoes seem to target similarly (e.g., suggesting that an attached AOP may dominate the biodistribution of peptidic cargoes).

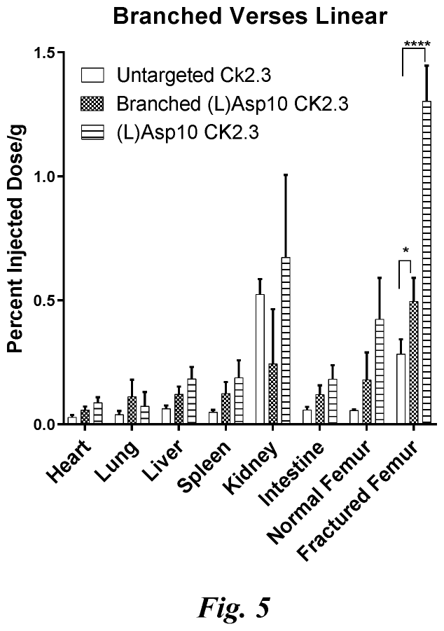

To explore the impact of peptide branching on payload targeting, the abilities of acidic oligo-aspartic acids constructed of either two chains of 5 aspartic acids each or a single linear chain of 10 aspartic acids to deliver the CK2.3 payload to fracture surfaces were compared. Provided herein is a graph of tissue vs. percent injected dose/g resulting therefrom (e.g., which shows the biodistribution of radio-iodinated CK2.3 coupled to branched or linear chains of 10 (L) aspartic acids relative to untargeted CK2.3) (e.g., FIG. 5). The biodistributions were determined 18 hours post-injection into ND-4 Swiss-Webster mice (n=5) bearing midshaft femur fractures 10 days post-fracture. As shown in FIG. 5, in some instances, linear peptides concentrated 2.7 times better on fracture surfaces than branched peptides. Moreover, since nontargeted CK2.3 displayed little uptake at the fracture site, nonspecific trauma-mediated deposition of CK2.3 may be dismissed as a major contributor to the accumulation of the acidic oligopeptide conjugate at the fracture site.

In some instances, the interaction of an AOP described herein with a bone fracture surface is mediated by its interaction with exposed calcium. For example, calcium can chelate when the proximal anionic charges are separated by a distance of 8.6 A. Recognizing that the lengths of the anionic side chains of the AOPs could determine this separation distance between negative charges, the targeting abilities of aspartic acid, glutamic acid and aminoadipic acid were compared, where the side-chain carboxyls extend from the peptide backbone by one, two, and three carbons, respectively, allowing an increasing separation between the anionic charges of the oligopeptide side chains.

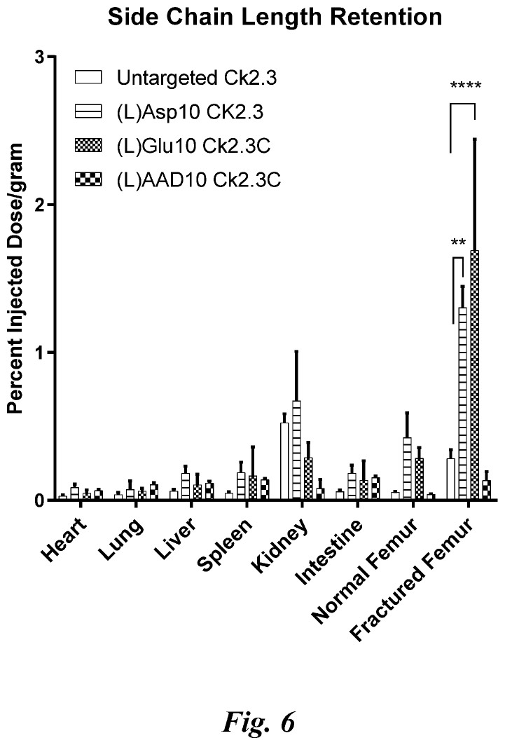

In some instances, FIG. 6 shows a graph of tissue vs. percent injected dose/gram (e.g., which shows the biodistribution of radio-iodinated CK2.3 coupled to linear chains of 10 (L) aspartic acids, 10 (L) glutamic acids, or 10 (L) aminoadipic acids relative to untargeted CK2.3). The biodistributions were determined 18 hours post-injection into ND-4 Swiss-Webster mice (n=5) bearing midshaft femur fractures 10 days post-fracture. In some instances, deca-glutamic and deca-aspartic acids exhibited the greatest uptake at the fracture site (e.g., with 6 times more accumulation than the nontargeted Ck2.3, and with aminoadipic acid promoting bone fracture retention not significantly different from nontargeted Ck2.3) (FIG. 6). In some instances, an AOP comprised of either glutamic or aspartic acids constitutes a peptide with optimal charge separation for calcium binding (e.g., explaining the branched peptide's reduction in binding). The fact that nature has primarily selected glutamic acids for its calcium binding functions in bone mineralization, together with the repeated observation that the synthesis of glutamic acid oligomers may be much more efficient than aspartic acid oligomers due to unwanted formation of aspartamides, prompted focus on the optimization of the glutamic acid oligomers for bone fracture-targeting efforts.

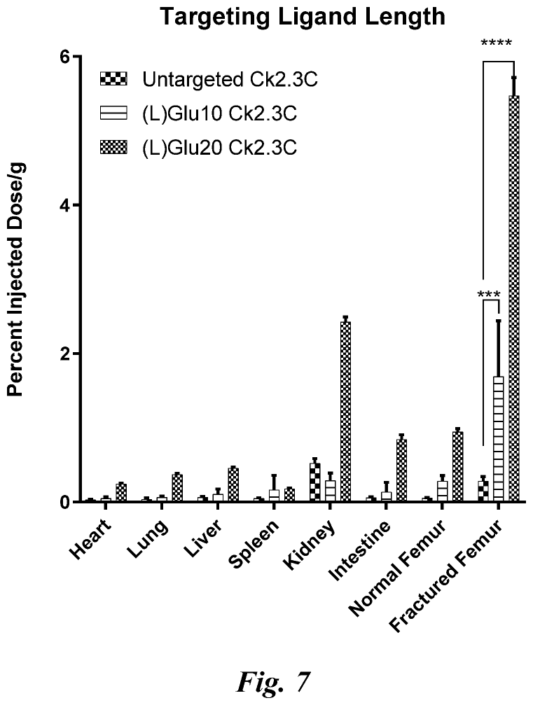

To explore the impact of oligopeptide length on the fracture-targeting ability of the molecule to be employed as Z of Formula (I), the abilities of oligo-glutamic acids of 10 or 20 amino acid lengths to deliver the same CK2.3 cargo to fractured femur surfaces were compared. FIG. 7 shows a graph of tissue vs. percent injected dose/g, which shows the biodistribution of radio-iodinated CK2.3 coupled to linear chains of 10 or 20 (L) glutamic acids relative to untargeted CK2.3. The biodistributions were determined 18 hours post-injection (e.g., into ND-4 Swiss-Webster mice (n=5) bearing midshaft femur fractures 10 days post-fracture). In some instances, CK2.3 tethered to the longer oligo-glutamic acid accumulates (e.g., 3.3 times) more at the fracture site than the shorter oligo-glutamic acid. While the affinities of unconjugated acidic oligopeptides seem to maximize at chain lengths of only 8 amino acids (see, e.g., Sekido et al., “Novel drug delivery system to bone using acidic oligopeptide: pharmacokinetic characteristics and pharmacological potential,” doi.org/10.3109/10611860108997922 (2001)), the observed increased affinity of the 20-mer over 10-mer observed could have arisen because more extensive binding to hydroxyapatite is required to retain a payload of the size of CK2.3 at the fracture surface. The improved localization of the CK2.3 payload with the 20-mer may also be in part due to a relative reduction in steric hinderance from the payload on the targeting ligand.

Studies by other groups have demonstrated that acidic oligopeptides are not readily orally bioavailable (see, e.g., Shaji et al., “Oral protein and peptide drug delivery,” Indian J. Pharmaceutical Sci. 70: 189-200 (2005)); however, in some instances, frequent injection discourages patient compliance and, thus, can be problematic. Provided in some instances herein is a long-lasting (e.g., injectable) formulation. In some instances, the affinities of acidic oligopeptides comprised of D- and L-amino acids for hydroxyapatite hare similar (Sekido et al. (2001), supra). In some instances, a linear oligo-glutamate chain composed of (e.g., poorly-metabolizable) D-glutamic acids (e.g., rather than a readily digestible chain comprised of L-glutamic acids) provided longer drug retention at the fracture surface. In some instances, the abilities of the D and L enantiomers of glutamic acid 20-mers to accumulate and persist at the fracture site were compared herein.

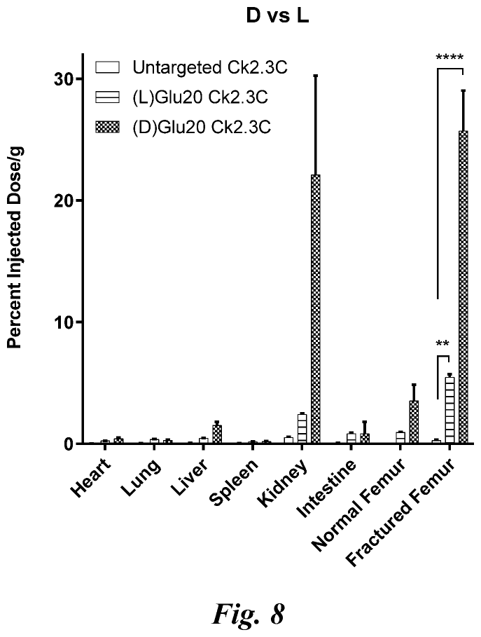

For example, FIG. 8 shows a graph of tissue vs. percent injected dose/g, which represents the biodistribution of radio-iodinated CK2.3 coupled to linear chains of 20 L- or D-glutamic acids relative to untargeted CK2.3. In some instances, the biodistribution is determined 18 hours post-injection (e.g., into ND-4 Swiss-Webster mice (n=5) bearing midshaft femur fractures 10 days post-fracture). For example, as shown in FIG. 8, the D enantiomer of Glum accumulated 4.7 times more than the L enantiomer at the fractured femur and 91.9 times as much as the nontargeted CK2.3.

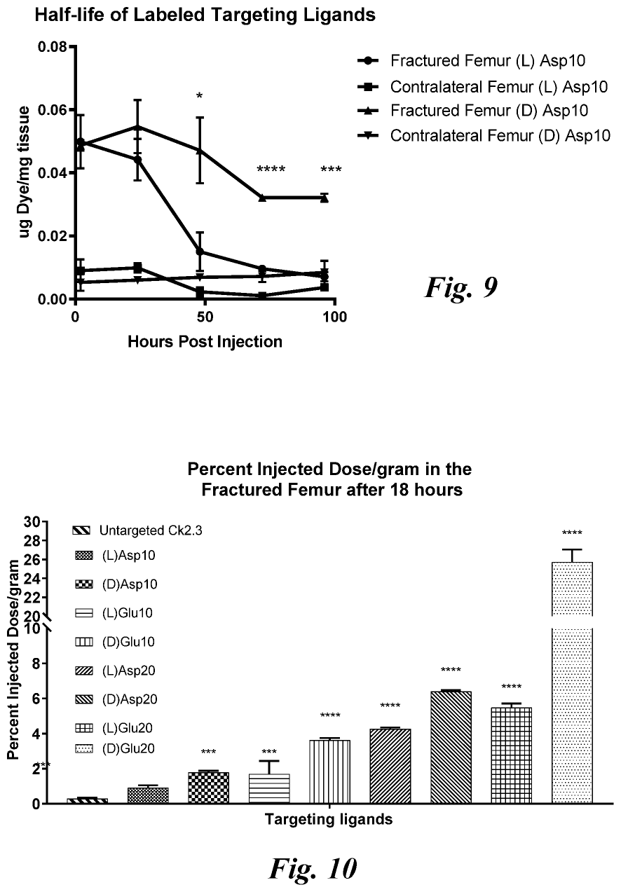

In some instances, the fluorescent dye, SO456, was attached to both D an L enantiomers of Asp₁₀ peptides (see FIG. 9). The accumulation of the differently labeled enantiomeric chains in both fractured and healthy contralateral femurs was quantified.

FIG. 9 is a graph of hours post-injection vs. μg dye/mg tissue, which shows the accumulation of S0456 (near-IR fluorophore) coupled to linear chains of 10 L- or D-aspartic acids in ND-4 Swiss-Webster mice bearing midshaft femur fractures 10 days post-fracture at different time points post-injection. In some instances, the accumulation of the labeled compounds in the healthy (e.g., undamaged contralateral femur) and the broken femur were quantified as the amount of labeled dye that was extracted from dissolved femurs post-mortem. For example, as shown in FIG. 9, the retention half-life of Asp₁₀ was estimated to be −35 hours, whereas that of (D)Asp₁₀ was projected to be over 100 hours. In some instances, the difference was slightly smaller than that detected with radiolabeled peptide payloads, which may be due to the shorter half-life of the peptide payloads relative to the fluorescent payload. In some instances, the enhanced stability resulted in prolonged clearance through the kidneys, for example, which may be because the slowly degradable D-isomer released more slowly from the bone and other tissues than the L-isomer.

In some instances, DE20 delivered significantly more cargo to the fracture site than (e.g., any of) the other targeting ligands provided herein (see FIG. 10). For example, FIG. 10 is a graph of targeting ligands vs. percent injected dose/gram, which shows the accumulation in fractured femurs of radio-iodinated CK2.3 coupled to different acidic oligopeptides relative to untargeted CK2.3. The biodistribution was determined 18 hours post-injection (e.g., into ND-4 Swiss-Webster mice (n=5) bearing midshaft femur fractures 10 days post-fracture). In some instances, the accumulation of the labeled compounds in the fractured femurs are reported as a percent of the injected dose per gram of tissue.

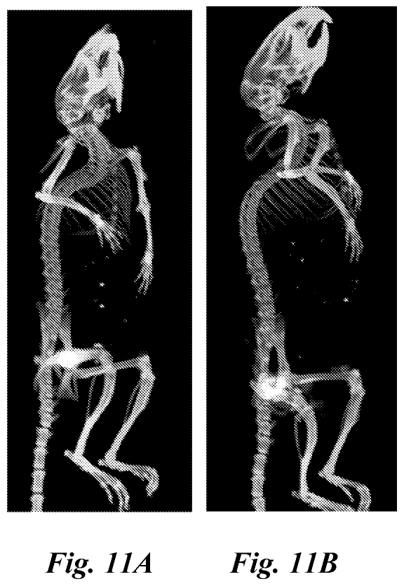

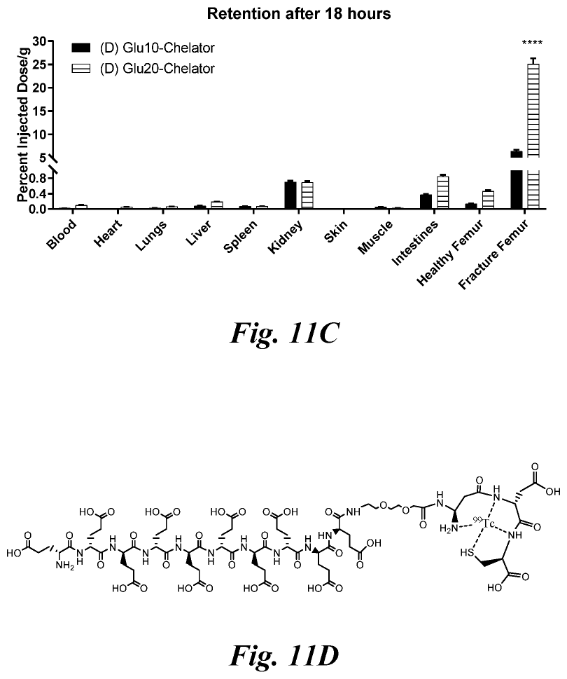

In some instances, to visually compare the biodistributions of the DE10 and DE20 oligopeptides, such as, for example, since the impact of extending the targeting ligand was greater than expected, SPECT/CT imaging of both oligopeptides is performed and their biodistributions are visually examined. For example, FIG. 11A is a single-photon emission computer tomography/computed tomography (SPEC/CT) image of the Tc chelator EC20 chelating 99Tc linked to DE10 acid (structure of EC20(D)Glu₁₀ chelating 99Tc is shown in FIG. 11D) and FIG. 11B is a SPEC/CT image of the Tc chelator EC20 chelating 99Tc linked to DE20 acid. As shown in FIGS. 11A and 11B, both acidic oligopeptides yielded highly resolved images with the targeted radio-imaging agents almost exclusively concentrated at the fracture site. Signal to volume ratios were greater than 10-fold higher in the fracture than in other adsorption sites such as the growth plates. Still, in some instances, the adsorption to the growth plates may limit patients to adults.

A second biodistribution analysis was also conducted. FIG. 11C, which is a graph of tissue vs. percent injected dose/g, shows the quantification of the accumulation of the labeled DE10 and DE20 compounds in the different tissues as a percent of injected dose per gram (n=10). The majority of signal was observed in the fracture callus of the femur and trace concentrations of drug were observed at sites of high bone turnover. In some instances, DE10 and DE20 had similar specificities, but DE20 had a longer retention in the bone. The results shown in FIG. 11C are very similar to those in FIG. 10 (i.e., DE20 accumulating ˜5 times more efficiently at the fracture site than DE10). Accordingly, in the studies described herein, DE20 exhibited the greatest fracture-targeting capacity of all ligands tested herein, and the DE20 oligopeptide displayed the greatest selectivity for fracture sites (e.g., of all targeting ligands tested herein).

The fracture healing efficacy of the targeted anabolic compounds hereof was also tested in vivo in murine subjects experiencing type I diabetes. FIG. 13 shows a graph of the agents tested (compounds comprising SEQ ID NO: 3, 5, 6, or 7 as compared to saline and insulin (controls)) vs. bone volume (BV). These results support the in vivo type I diabetic fracture healing efficacy of targeted anabolic conjugates on male IGS-1 fracture-bearing mice (n=10) after four weeks was higher than (i.e. more therapeutically effective than) the controls.

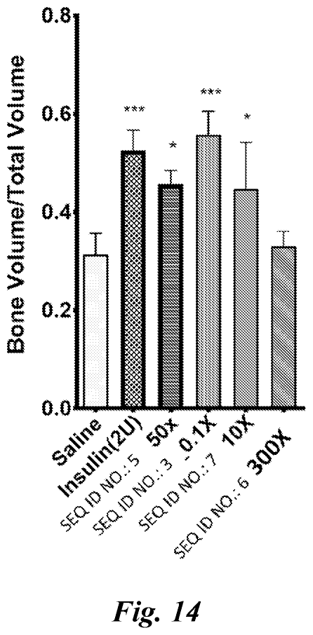

FIG. 14 shows a graph of agents tested (compounds comprising SEQ ID NO: 3, 5, 6, or 7 as compared to saline and insulin (controls)) vs. bone volume/total volume (BV/TV). These results display the in vivo type I diabetic fracture healing efficacy of targeted anabolic conjugates on male IGS-1 fracture-bearing mice (n=10) after four weeks.

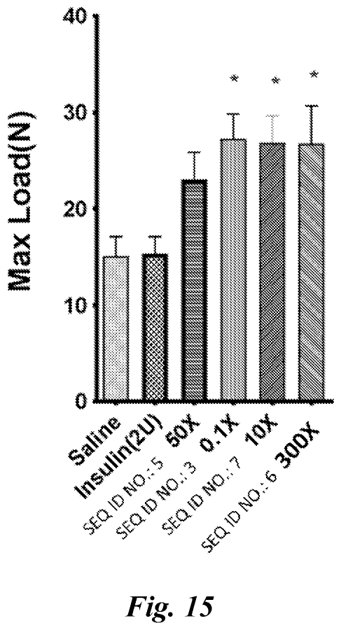

FIG. 15 shows is a graph of agents tested (compounds comprising SEQ ID NO: 3, 5, 6, or 7 as compared to saline and insulin (controls)) vs. max load (N), which shows the in vivo type I diabetic fracture healing efficacy of targeted anabolic conjugates on male IGS-1 fracture-bearing mice (n=10) after four weeks. In some instances, max load represents the maximum force the healed femur withstood before it refractured in a post-mortem four-point bend analysis. In some instances, max load is a measure of how strong the bone is at the site of fracture repair.

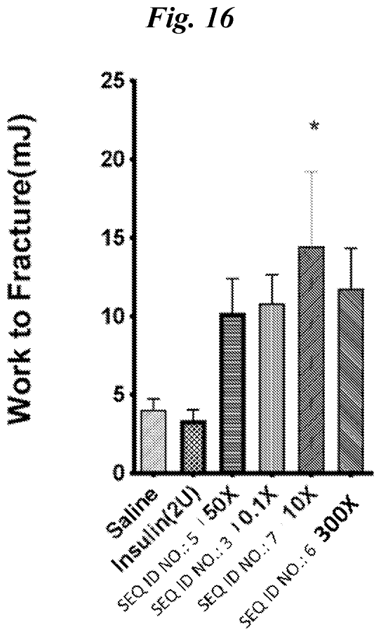

FIG. 16 shows a graph of agents tested (compounds comprising SEQ ID NO: 3, 5, 6, or 7 as compared to saline and insulin (controls)) vs. work to fracture (mJ), which shows the in vivo type I diabetic fracture healing efficacy of targeted anabolic conjugates on male IGS-1 fracture-bearing mice (n=10) after four weeks. In some instances, work to fracture represents the total amount of energy absorbed by the healed femur before it refractured in a post-mortem four-point bend analysis. In some instances, work to fracture is a measure of how strong the bone is at the site of fracture repair.

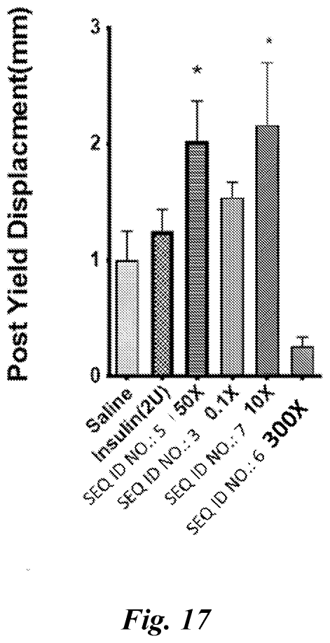

FIG. 17 is a graph of agents tested (compounds comprising SEQ ID NO: 3, 5, 6, or 7 as compared to saline and insulin (controls)) vs. post-yield displacement (mm), which shows the in vivo type I diabetic fracture healing efficacy of targeted anabolic conjugates on male IGS-1 fracture-bearing mice (n=10) after four weeks. Here, post-yield displacement is a measure of how brittle a bone is (e.g., diabetes typically makes the bone more brittle). In some instances, a compound provided herein reduced the brittleness of bone (e.g., FIG. 17).

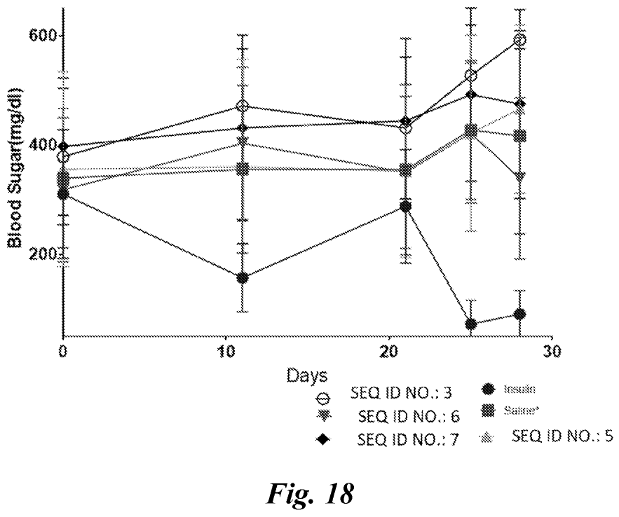

FIG. 18 is a graph of days vs. blood sugar (mg/dl), which shows the average blood sugar levels of the type I diabetic mice during the four-week treatment period of compounds comprising SEQ ID NO: 3, 5, 6, or 7 (and the insulin and saline controls). In some instances, longitudinal tracking of blood sugar of the different treated groups showed that only insulin had a significant impact on hyperglycemia, such as, for example, suggesting that the effect of a compound provided herein is not through blood sugar metabolism. In some instances, the attachment of a targeting ligand to SEQ ID NO: 5's C-terminus interferes with SEQ ID NO: 5's glucose-modulating effects. In some instances, the micro-CT scans of the callus illustrated that all the treatments except for SEQ ID NO: 7 favored an endochondral ossification.

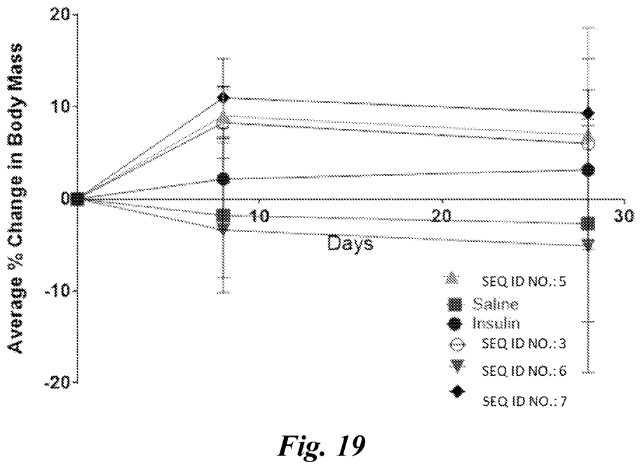

FIG. 19 is a graph of days vs. average % change in body mass, which shows the mean weight change for the type I diabetic fracture mice treated groups throughout treatment. In some instances, the saline mice lost weight (as is expected with diabetes), as did the mice treated with an agent comprising SEQ ID NO: 6 (a targeted conjugate of dasatinib (e.g., D10-ester-dasatinib)), for example, which exhibited some toxicity at higher doses. In some instances, SEQ ID NO: 6 improved strength without improving the callus mineralization (e.g., which may be due to its senolytic effects).

Accordingly, in some instances, a compound provided herein is non-toxic (e.g., regarding gross weight changes). Furthermore, administration of a compound provided herein can improve mineralization and strength (e.g., even though the hyperglycemic state was not controlled for in any group except for the insulin-treated group). In some instances, hyperglycemia is toxic to stem cells.

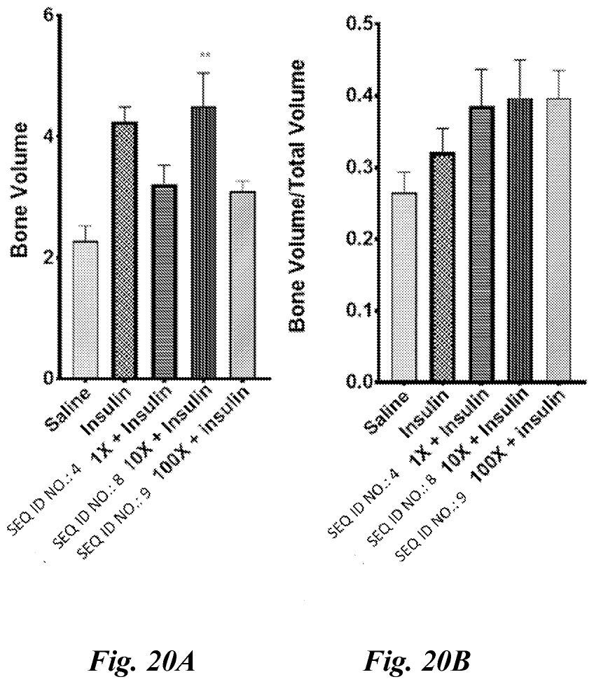

In certain embodiments, insulin is administered with the compounds and compositions provided herein. As supported by the data shown in FIGS. 20A-22C, the compounds hereof can significantly improve healing relative to the group treated with only insulin.

FIG. 20A shows a graph of agents hereof (comprising SEQ ID NO: 4, 8, or 9) tested as compared to saline and insulin (controls) vs. bone volume, which show the in vivo type I diabetic fracture healing efficacy of targeted anabolic conjugates on male IGS-1 fracture-bearing mice (n=10) after four weeks. As shown in FIG. 20A, bone volume represents the bone volume of the 100 thickest micro-CT slides of the fracture callus and is a measure of how much bone has mineralized at the site of fracture repair. FIG. 20B is a graph of such agents tested (as compared to saline and insulin as controls) vs. bone volume/total volume (BV/TV), which show the in vivo type I diabetic fracture healing efficacy of targeted anabolic conjugates on male IGS-1 fracture-bearing mice (n=10) after four weeks. Here, BV/TV represents the bone volume divided by total volume of the 100 thickest micro-CT slides of the fracture callus and is a measure of how dense the bone is at the site of fracture repair.

In some instances, as shown in FIGS. 20A and 20B, the compounds provided herein improved mineralization of the callus (e.g., as compared to treatment with insulin alone). In some instances, such compounds increased fracture callus density (e.g., more so than insulin alone).

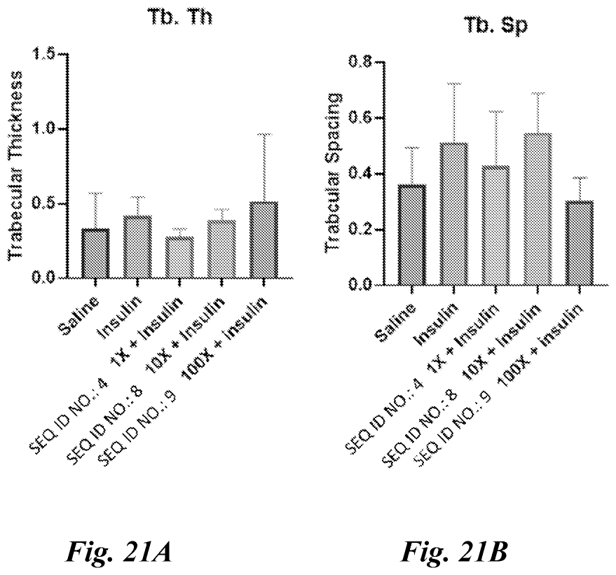

FIG. 21A is a graph of agents (e.g., SEQ ID NO: 4, 8, or 9) tested in conjunction with insulin administration (compared to saline and insulin alone) vs. trabecular thickness, which shows the in vivo type I diabetic fracture healing efficacy of targeted anabolic conjugates on male IGS-1 fracture-bearing mice (n=10) after four weeks. FIG. 21B is a graph of agents (e.g., SEQ ID NO: 4, 8, or 9) tested in conjunction with with insulin administration (compared to saline and insulin alone) vs. trabecular spacing, which shows the in vivo type I diabetic fracture healing efficacy of targeted anabolic conjugates on male IGS-1 fracture-bearing mice (n=10) after four weeks 0.

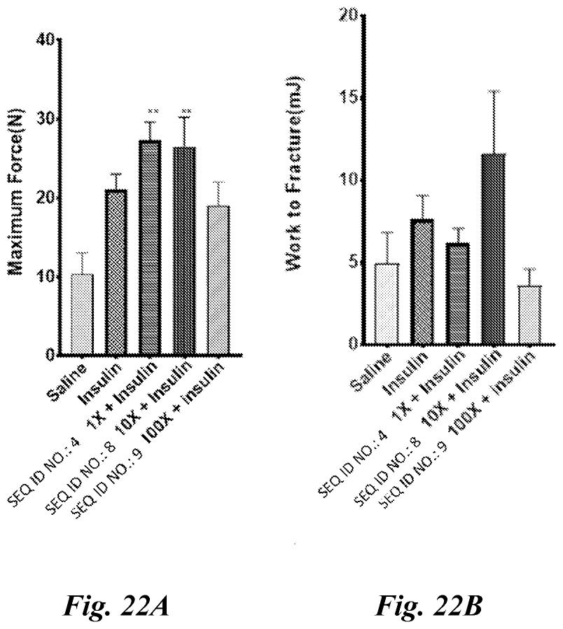

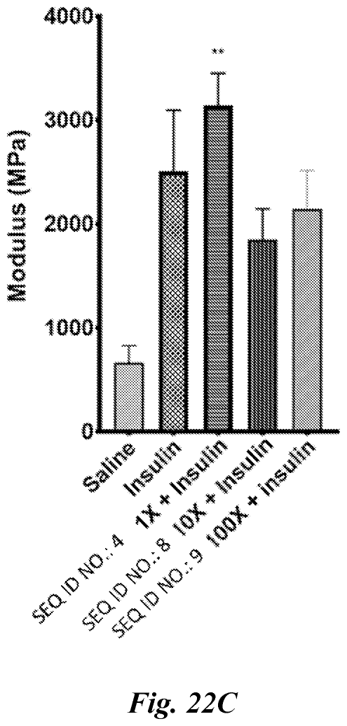

FIG. 22A is a graph of agents (e.g., SEQ ID NO: 4, 8, or 9) tested with insulin (compared to saline and insulin alone) vs. maximum force (N), which shows the in vivo fracture healing efficacy of targeted anabolic conjugates on male IGS-1 fracture-bearing mice (n=10) after four weeks. Here, maximum force represents the maximum force the healed femur withstood before it refractured. FIG. 22B displays a graph of agents (e.g., SEQ ID NO: 4, 8, or 9) tested in conjunction with insulin administration (compared to saline and insulin alone) vs. work to fracture (mJ), which shows the in vivo fracture healing efficacy of targeted anabolic conjugate on male IGS-1 fracture-bearing mice (n=10) after four weeks. Work to fracture represents the total amount of energy absorbed by the healed femur before it refractured and can be a measure of how strong the bone is at the site of fracture repair. FIG. 22C is a graph of agents (e.g., SEQ ID NO: 4, 8, or 9) tested in conjunction with insulin administration (compared to saline and insulin alone) vs. modulus (MPa), which shows the in vivo fracture healing efficacy of targeted anabolic conjugates on male IGS-1 fracture-bearing mice (n=10) after four weeks. Stiffness is a measure of Young's modulus of the healed femur in a postmortem 4-point bend analysis and can be a measure of how resistant a bone is to deformation. In some instances, a compound provided herein (comprising, for example, abaloparatide (SEQ ID NO: 2 which is within SEQ ID NO: 3) and SEQ ID NO: 8) (e.g., significantly) improves strength compared to the insulin control. In some instances, a compound provided herein (e.g., abaloparatide (SEQ ID NO: 2) and SEQ ID NO: 8) improves strength and mineralization more than insulin alone.

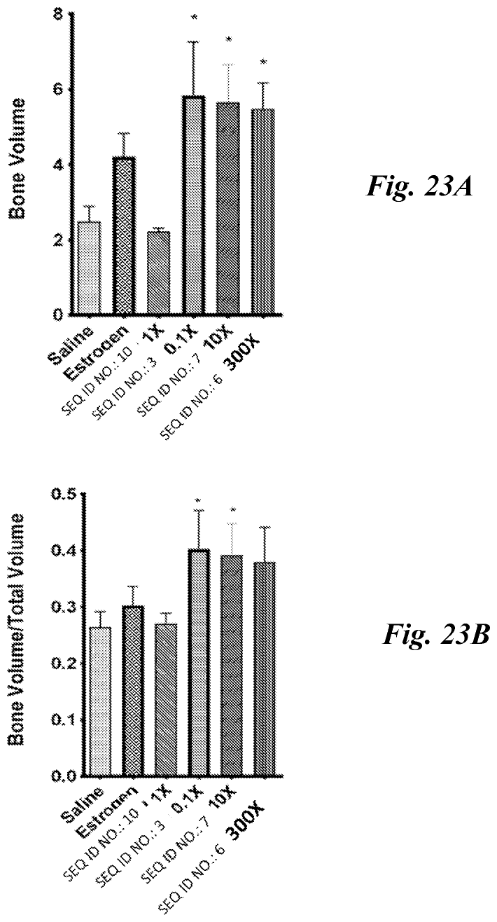

In some instances, a compound provided herein improves the mineralization and density of the fracture callus (e.g., relative to saline (e.g., and more so than estrogen replacement)) (FIGS. 23A-B).

FIG. 23A is a graph of agents (e.g., comprising SEQ ID NO: 3, 6, 7, or 10) tested vs. bone volume, which shows the in vivo postmenopausal osteoporotic fracture healing efficacy of targeted anabolic conjugate on female ovariectomized Swiss Webster fracture-bearing mice (n=9) after four weeks. As referred to in FIG. 23A, bone volume represents the bone volume of the 100 thickest micro-CT slices of the fracture callus and is a measure of how much bone has mineralized at the site of fracture repair. FIG. 23B is a graph of agents tested vs. bone volume/total volume (BV/TV), which shows the in vivo postmenopausal osteoporotic fracture healing efficacy of targeted anabolic conjugate on female ovariectomized Swiss Webster fracture-bearing mice (n=9) after four weeks. In FIG. 23B, BV/TV represents the bone volume divided by total volume of the 100 thickest micro-CT slices of the fracture callus and is a measure of how dense the bone is at the site of fracture repair.

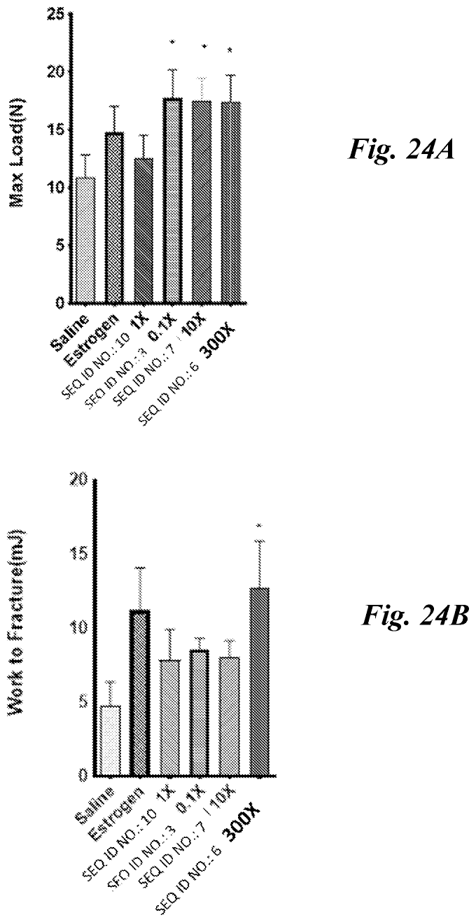

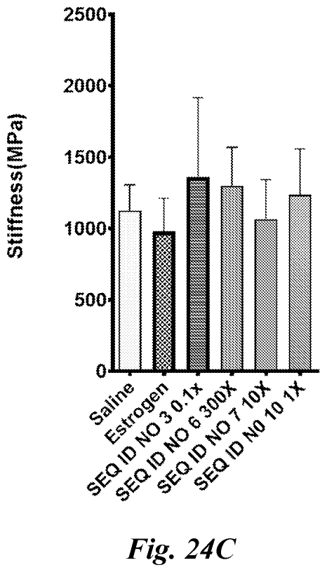

In some instances, a compound provided herein improves strength of a femur (see, e.g., FIGS. 24A-C).

FIG. 24A is a graph of agents (comprising, for example, SEQ ID NO: 3, 6, 7 or 10) tested (as compared to saline or estrogen as controls) vs. max load (N), which shows the in vivo postmenopausal osteoporosis fracture healing efficacy of targeted anabolic conjugate on female OVX Swiss Webster fracture-bearing mice (n=9) after four weeks. In FIG. 24A, max load represents the maximum force the healed femur withstood before it refractured.

FIG. 24B is a graph of agents (comprising, for example, SEQ ID NO: 3, 6, 7 or 10) tested (as compared to saline or estrogen as controls) vs. work to fracture (mJ), which shows the in vivo postmenopausal osteoporosis fracture healing efficacy of targeted anabolic conjugates on female OVX Swiss Webster fracture-bearing mice (n=9) after four weeks. In FIG. 24B, work to fracture represents a measure of how strong the bone is at the site of fracture repair.

FIG. 24C is a graph of agents (comprising, for example, SEQ ID NO: 3, 6, 7 or 10) tested (as compared to saline or estrogen as controls) vs. stiffness (MPa), which shows the in vivo postmenopausal osteoporosis fracture healing efficacy of targeted anabolic conjugates on female OVX Swiss Webster fracture-bearing mice (n=9) after four weeks. In FIG. 24C, stiffness is a measure of Young's modulus of the healed femur in a postmortem 4-point bend analysis and can be used as a measure of how resistant a bone is to deformation.

In some instances, a compound provided herein (e.g., significantly) improves the healing of fracture femurs (e.g., in a hypoestrogenic state described herein (e.g., far better than controlling the loss of estrogen with estrogen replacement). In some instances, a compound provided herein is administered to osteoporotic patients with bone fractures.

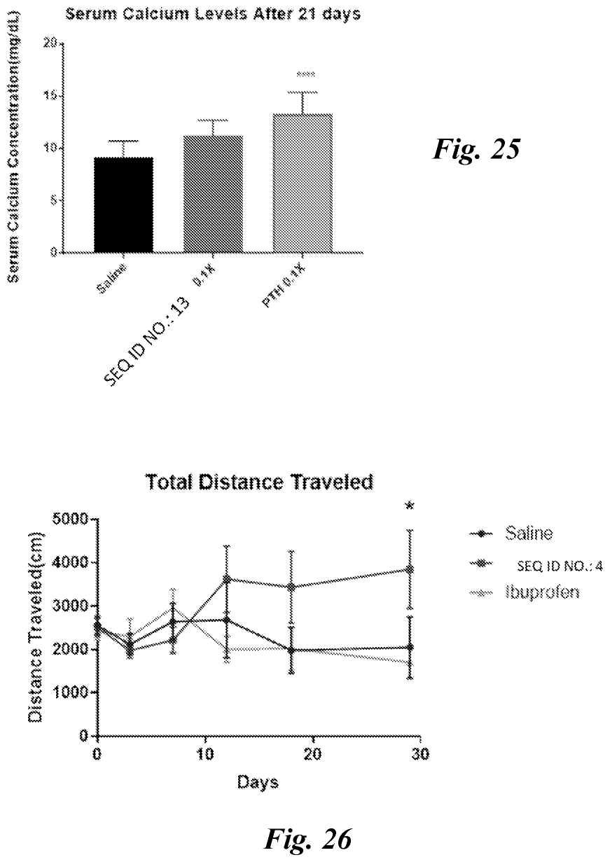

FIG. 25 is a graph of agents (comprising, for example, SEQ ID NO: 3, 6, 7 or 10) tested (as compared to saline or estrogen as controls) vs. serum calcium concentration (mg/dl), which shows the effect of 21 days of treatment on serum calcium in a Swiss Webster mouse with a midshaft femur fracture model. In some instances, using a targeted anabolic described herein (e.g., as opposed to free anabolics) is beneficial for treating bone fracture, such as, for example, because targeted anabolics hereof can limit the effects on the regulation of calcium metabolism that occurs in the kidneys for parathyroid hormones.

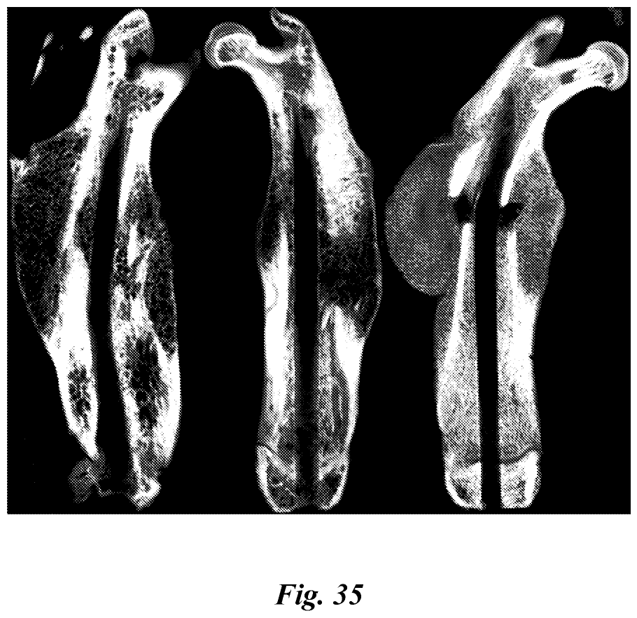

In some instances, elevated accumulation of SEQ ID NO: 4 at the fracture site improves fracture healing (e.g., FIGS. 26-30). In some instances, femur fracture healing times following administration of a starting dose calculated by allometric scaling of the human dose prescribed for abaloparatide (SEQ ID NO: 2) treatment of osteoporosis are compared. In some instances, CT images show that bone deposition imaged three weeks after initiation of treatment with non-targeted abaloparatide is concentrated on the periphery of the fracture, with minimal density bridging the opposing calluses (see, e.g., FIG. 35).

In some instances, such as, for example, following three weeks of therapy with a targeted abaloparatide (SEQ ID NO: 4), bone density is distributed (e.g., more) evenly across the fractured area subsequent to administration of a compound provided herein (e.g., with the overall size of the callus also exceeding that in the abaloparatide-treated mice). Bone morphometric analyses confirmed a 1.5-fold increase in the ratio of mineralized volume (bone volume; BV) to total bone volume (total volume; TV) in the SEQ ID NO: 4-treated mice. More detailed morphometric analysis further revealed that this increase in bone density is primarily due to a reduction in trabecular spacing, rather than an increase in trabecular thickness in the SEQ ID NO: 4-treated femurs.

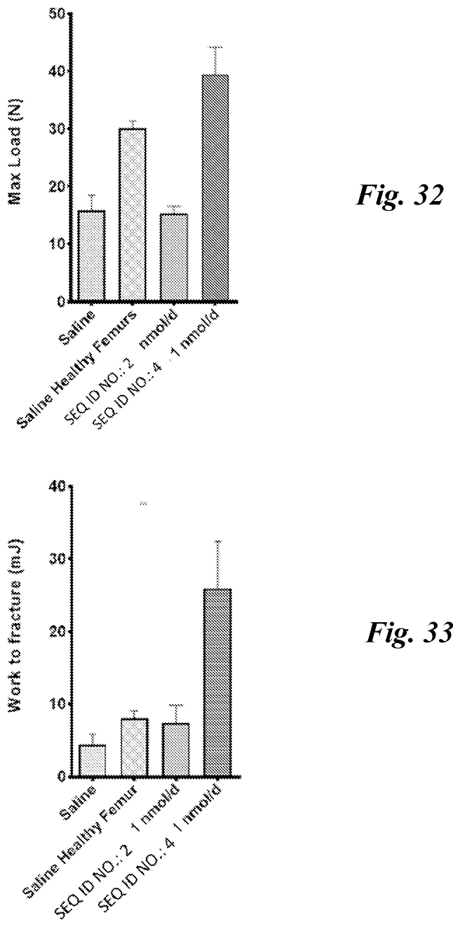

In some instances, such as, to establish whether the observed increase in bone deposition translates into an improvement in mechanical properties, the force required to bend the healed femurs until they fractured again was measured. In some instances, the SEQ ID NO: 4-treated fractures (e.g., of mice with midshaft femoral factures) were observed to sustain a 2.5-fold greater maximum load than the free abaloparatide-treated femurs (FIG. 32).

In some instances, the force to fracture in the SEQ ID NO: 4-treated mice exceeded that required to fracture the contralateral healthy femurs in the same mice or similar, unmodified femurs in saline-treated mice. This suggests the repaired femurs at this time point were 30% stronger than the original unbroken femurs. In some instances, the work to fracture (e.g., in similar mechanical studies) was averaged 3.5-fold higher in the SEQ ID NO: 4-treated femurs than in the abaloparatide-treated femurs (FIG. 33) of mice with midshaft femoral factures.

Taken together, these data demonstrate that D-Glu₂₀-mediated accumulation of abaloparatide on a fracture surface greatly facilitates the healing process, returning fractured femurs to full mechanical strength more rapidly than femurs in saline or abaloparatide-treated mice.

In some instances, SEQ ID NO: 4 is more effective (e.g., at improving bone healing) than SEQ ID NO: 3.

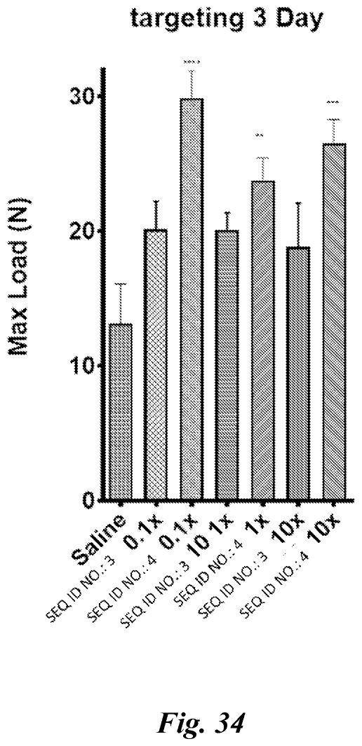

While SEQ ID NO: 3, in some instances, is better than abaloparatide, its clinical relevance may be dramatically improved if patients required fewer doses. The length of the targeting oligopeptide was increased to determine if it increased the drug's affinity for broken bone sufficiently that retention of the anabolic payload at the fracture site would be prolonged and less frequent dosing may be required. Mice were doses once every three days, rather than daily. Visual inspection of CT scans revealed that more bone is deposited at three weeks post-fracture in mice treated with targeted abaloparatide vs. non-targeted abaloparatide. When comparing the maximum load achieved before breakage by abalode 10 vs. abalode 20, the trend demonstrated 20-30% greater strength. When each drug was compared to saline, SEQ ID NO: 4 was significantly higher, whereas the SEQ ID NO: 3 lacked significance when dosed every three days. The results are shown in FIG. 34, which is a graph of treatment (saline, SEQ ID NO: 3 (0.1 nmol/mg (0.1×), 1 nmol/mg (1×), and 10 nmol/mg (10×), and SEQ ID NO: 4 (0.1 nmol/mg (0.1×), 1 nmol/mg (1×), and 10 nmol/mg (10×)) vs. max load (N) of mice with midshaft femoral factures.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

While certain embodiments of the present disclosure have been shown and described herein, it will be apparent to those skilled in the art that such embodiments are provided by way of example only. It is not intended that the claimed invention be limited by the specific examples provided within the specification.

The descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein, which depend upon a variety of conditions and variables. It should be understood that various alternatives to the embodiments of the invention described herein may be employed in practicing the invention. It is, therefore, contemplated that the invention shall also cover any such alternatives, modifications, variations or equivalents. It is intended that the following claims define the scope of the invention and that methods and structures within the scope of these claims and their equivalents be covered thereby.

### Certain Definitions

As used herein and in the appended claims, the singular forms “a,” “and,” and “the” include plural referents unless the context clearly dictates otherwise. Thus, for example, reference to “a compound” includes a plurality of such compounds. When ranges are used herein for physical properties, such as molecular weight, or chemical properties, such as chemical formulae, all combinations and sub-combinations of ranges and specific embodiments therein are intended to be included. The term “about,” when referring to a number or a numerical range, means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude an embodiment of any compound, composition, method, process, or the like that may “consist of” or “consist essentially of” the described features. The invention illustratively described herein may be suitably practiced in the absence of any element(s) or limitation(s), which is/are not specifically disclosed herein.

“Percent (%) sequence identity” with respect to a reference to a sequence is defined as the percentage of amino acid or nucleic acid residues, respectively, in a candidate sequence that are identical with the residues in the reference sequence, after aligning the sequences and introducing gaps, if necessary, to achieve the maximum percent sequence identity and not considering any conservative substitutions as part of the sequence identity. Alignment for purposes of determining percent sequence identity can be achieved in various ways that are within the skill of the art, for instance, using publicly available computer software. For example, determination of percent identity or similarity between sequences can be done, for example, by using the GAP program (Genetics Computer Group, software; now available via Accelrys on http://www.accelrys.com), and alignments can be done using, for example, the ClustalW algorithm (VNTI software, InforMax Inc., Gaithersburg, MD). Further, a sequence database can be searched using the nucleic acid or amino acid sequence of interest. Algorithms for database searching are typically based on the BLAST software (Altschul et al., 1990), but those skilled in the art can determine appropriate parameters for aligning sequences, including any algorithms needed to achieve maximal alignment over the full length of the sequences being compared. In some embodiments, the percent identity can be determined along the full-length of the nucleic acid or amino acid sequence.

As described herein, certain compounds of the present disclosure can contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group can have a suitable substituent at each substitutable position of the group, and when more than one position in any given structure may be substituted with more than one substituent selected from a specified group, the substituent can be either the same or different at each position. Combinations of substituents envisioned are preferably those that result in the formation of stable or chemically feasible compounds.

As used herein, the terms “patient,” “subject,” and “individual” are used interchangeably. None of the terms require the supervision of medical personnel. For example, administering to an individual includes the individual administering the therapeutic agent to themselves, as well as a medical professional administering the therapeutic agent to the individual.

The term “radical” as used herein refers to a fragment of a molecule, wherein that fragment has an open valence which is an attachment point for bond formation. A monovalent radical has one open valence such that it can form one bond with another chemical group. In some embodiments, a radical of a molecule (e.g., a radical of a folate receptor binder) as used herein is created by removal of one hydrogen atom from that molecule to create a monovalent radical with one open valence at the location where the hydrogen atom was removed. Where appropriate, a radical can be divalent, trivalent, etc., wherein two, three or more hydrogen atoms have been removed to create a radical which can bond to two, three, or more chemical groups. Where appropriate, a radical open valence can be created by removal of other than a hydrogen atom (e.g., a halogen atom), or by removal of two or more atoms (e.g., a hydroxyl group), as long as the atoms removed are a small fraction (about 20% or less of the atom count) of the total atoms in the molecule forming the radical.

The terms “treat,” “treating,” or “treatment” include reducing, alleviating, abating, ameliorating, relieving, or lessening the symptoms associated with a bone fracture, diabetes, osteoporosis in either a chronic or acute therapeutic scenario.

The terms and expressions, which have been employed, are used as terms of description and not of limitation. In this regard, where certain terms are defined under “Certain Definitions” and are otherwise defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Certain Definitions,” are used in the “Detailed Description,” such use is solely for ease of reference and is not intended to limit any disclosure made in one section to that section only; rather, any disclosure made under one subheading is intended to constitute a disclosure under each and every other subheading.

### EXAMPLES

The following examples serve to illustrate the present disclosure. The examples are not intended to limit the scope of the claimed invention in any way.

All statistical analyses were performed with GraphPad Prism (version 8.0; GraphPad Software, CA). Data are displayed as mean±standard deviation. In the figures, levels of statistical significance are denoted with asterisks according to the following definition: *p<0.05; **p<0.01; ***p<0.001; ****p<0.0001. Statistical analysis was performed using a one-way analysis of variance (ANOVA) and a Dunnett's post-hoc analysis with adjusted significance reported at the P value of 0.05. For FIGS. 4-8 a Bonferroni post-hoc analysis was performed instead of a Dunnet's post-hoc analysis. FIG. 9 was analyzed with a two-way ANOVA with a Dunnett's post-hoc analysis.

### Example 1: Synthesis of Peptidic Payloads

All payloads (e.g., FIG. 1A) were synthesized in a solid-phase peptide synthesis vial under a stream of argon. Wang resin (0.6 mmol/g) was loaded with 3-fold excess of the first amino acid (cysteine), HOBt-Cl and DIC for 4 hours in 9:1 v/v CH₂Cl₂/dimethylformamide (DMF) using catalytic amounts of 4-dimethylaminopyridine (DMAP). The resin was then capped with two equivalents of acetic anhydride and pyridine for 30 minutes to block any unreacted hydroxyl groups on the resin. These steps were followed by three washes with methylene chloride (DCM) and DMF, consecutively.

After each coupling reaction, 9-fluorenylmethoxycarbonyl (Fmoc) groups were removed by two 10-minute incubations with 20% (v/v) piperidine in DMF. The resin was then washed twice with DMF prior to adding the next amino acid. Each amino acid was reacted in 3-fold excess 2-(1H-benzotriazol-1-yl)-1,1,3,3-tetramethyluronium hexafluorophosphate (HBTU)/N-methylmorpholine (NMM) for 30 minutes, followed by a double coupling with 3-fold excess benzotriazol-1-yl-oxytripyrrolidinophosphonium hexafluorophosphate (PyBOP)/N-methylmorpholine (NMM) for 30 minutes. All amino acids were added according to the conditions above. Standard Fmoc-protected amino acids with acid-sensitive side chain protecting groups were used, unless otherwise noted. Thereafter, tyrosine or the peptide sequence shown in Table 1 was added onto the peptide using the solid-phase procedures listed above using an automated peptide synthesizer (Focus XC, AAPPTec). Upon synthesis completion, the terminal Fmoc was removed using the aforementioned conditions, after which the resin was washed three times with DMF, three times with DCM, and twice with methanol, and then dried with argon gas.

The dried resin with the peptide was cleaved using 95:2.5:2.5 trifluoroacetic acid/water/triisopropylsilane and excess TCEP for 2 hours. The peptide was then precipitated from the cleavage solution using 10 times the volume of cold diethyl ether. The solution was spun at 2,000 relative centrifugal force (RCF) for five minutes and then decanted. The pellet was then desiccated and submitted to analytical liquid chromatography-mass spectrometry (1220 LC; 6130 MS, Agilent) for confirmation of synthesis. The crude peptide was dissolved in a mixture of DMF and water and purified via preparative reversed-phase high-performance liquid chromatography (1290, Agilent, Santa Clara, CA). A C-18 column with a 0-50% ammonium acetate:acetonitrile mobile phase for 40 minutes was used to purify the 2,2,6,6-tetramethylpiperidine (TMP). The fraction that contained only pure payloads as assessed by analytical liquid chromatography-mass spectrometry (1220 LC; 6130 MS, Agilent, Santa Clara, CA) was lyophilized (FreeZone, LABCONCO, Kansas City, MO) and stored as lyophilized powder at −20° C. until it was coupled with targeting ligands.

The following substitutions were introduced into residues 1-46 of parathyroid hormone-related protein (PTHrP) (SEQ ID NO: 1): Glu22, Glu25, Leu23, Leu28, Leu31, Lys26, Lys30, and Aib29 in accordance with methods known in the art. These substitutions enhance peptide stability, induce greater bone density in patients with osteoporosis, and expand the window of maximal anabolic activity without increasing toxicity. To maximize signaling of the anabolic peptide upon adsorption to exposed hydroxyapatite, the C-terminus of this PTHrP fragment was conjugated to a linear peptide of 20 D-glutamic acid (E) residues (D-Glu₂₀ or DE20) using standard solid phase peptide chemistry, yielding a final fusion protein (SEQ ID NO: 4) in 19% overall yield and final purity of 94% as evidenced by high pressure liquid chromatography (HPLC) and mass spectrometry.

### Example 2: Synthesis of (Linear) Osteotropic Peptides

Targeting ligand peptides were all synthesized to achieve the appropriate length, amino acid composition and enantiomeric stereochemistry, as indicated by their names according to the solid phase synthesis methods described above. While still on the resin, the N-terminal amines were deprotected as described above, and the resin was reacted in DMF with 3-fold maleimide propionic acid, 3-fold excess benzotriazol-1-yloxytripyrrolidinophosphonium hexafluorophosphate (PYBOP), HOBt-Cl and 5-fold excess N,N-diisopropylethylamine (DIPEA) for 4 hours. The peptides were then coupled to the cysteine-containing peptides using maleimide chemistry in phosphate-buffered saline (PBS) containing 10-fold excess tris(2-carboxyethyl)phosphine (TCEP) for 24 hours at room temperature. The targeting payload conjugates were then cleaved, deprotected, and purified as described above.

### Example 3: Synthesis of (Branched) Osteotropic Peptides

Briefly, branched targeting ligands were synthesized using solid-phase peptide synthesis under a stream of argon. 2-chlorotrityl resin (0.6 mmol/g) was loaded at 0.6 mmol/g with Nα,Nε-di-Fmoc-L-lysine for 60 minutes in DCM and DIPEA. The resin was then capped with four washes of MeOH, followed by three washes with DCM and DMF, consecutively. The branched chain was then synthesized as described above. The N-terminal Fmoc was retained and the peptide was subjected to a soft cleavage in 1:1:8 mixture of acetic acid/tetrafluoroethylene (TFE)/DCM for 30 minutes. The cleavage solution was evaporated under reduced pressure and the terminal carboxylic acid was conjugated with 3-fold excess N-(2-aminoethyl)maleimide, 3-fold excess PYBOP and HOBt-Cl and 5-fold excess DIPEA in DCM for 4 hours. The acid-sensitive protecting groups were then deprotected by a two-hour incubation in 95:2.5:2.5 trifluoroacetic acid/water/triisopropylsilane. The peptide was then precipitated with 10 volumes of cold diethyl ether, and the terminal Fmoc was deprotected by a 15-minute incubation with 20% (v/v) piperidine in DMF followed by a precipitation in cold diethyl ether. The resulting crude product was purified via preparative reversed-phase high-performance liquid chromatography (1290, Agilent) as described above. Finally, the purified targeting ligand was conjugated with different payloads via maleimide coupling also as described above.

### Example 4: Synthesis of Mono-Bisphosphonate Targeting Ligands

Alendronic acid was dissolved in sodium hydroxide and then diluted in 2-(N-morpholino)ethanesulfonic acid (MES) buffer, and the pH was reduced to 5 with HCl. Three equivalents of 3-maleimidopropionic acid were pre-activated with four equivalents of 1-ethyl-3-(3-dimethylaminopropyl)cabodiimide (EDC). The reaction was stirred overnight at 40° C. and the crude product was purified by preparative reversed-phase high-performance liquid chromatography (1290, Agilent, Santa Clara, CA) on a C-18 column using a 0-25% ammonium acetate/acetonitrile mobile phase for 40 minutes.

The fractions that contained only pure maleimide product as analyzed by analytical liquid chromatography-mass spectrometry (1220 LC; 6130 MS, Agilent) were lyophilized and stored at −20° C. until required for coupling with payloads via maleimide coupling as described above.

### Example 5: Synthesis of Tri-Bisphosphonate Targeting Ligands

Di-tert-butyl-2,2′-((3-amino-2-(2-(2-(tert-butoxy)-2-oxoethoxy)ethyl)pentane-1,5-diyl)bis(oxy))diacetate was reacted with 1.5 equivalents of 3-maleimidopropionic acid, 4 equivalents of N,N′-dicyclohexylcarbodiimide (DCC), and 3 equivalents of DIPEA in DCM at 45° C. for 24 hours. The dicyclohexyl urea (DCU) precipitate was filtered out and the volume reduced under low pressure. The product was purified via flash chromatography and the carboxylic acids were deprotected in 50:50 TFA/DCM for 30 minutes. The solvent was removed under reduced pressure and the resulting 2,2′-((2-(3-(carboxymethoxy)-1-(3-(2,5-dioxo-2,5-dihydro-1H-pyrrol-1-yl)propanamido)propyl)butane-1,4-diyl)bis(oxy))diacetic acid was reacted with 12 equivalents of alendronic acid plus 12 equivalents of EDC in MES buffer at pH 4.5 for 24 hours at 45° C. The resulting crude product was purified via preparative reversed-phase high-performance liquid chromatography (1290, Agilent, Santa Clara, CA) and the purified targeting ligand was conjugated with different payloads via maleimide coupling as described above. The structure of a tri-bisphosphonate is shown in FIG. 12. R can represent any peptide or small molecule.

### Example 6: Synthesis of Polyphosphate Targeting Ligands

A phosphate glass polymer of 45 phosphates was dissolved in 100 mM MES at a concentration of 10 mM. Sufficient EDC was then added to achieve a 100 mM concentration, and then three equivalents of DIPEA followed by five equivalents of N-(2-aminoethyl)-maleimide were added. The purified targeting ligand was conjugated with different payloads via maleimide coupling as described above.

### Example 7: Synthesis of 99mTc Chelator Molecules

^{99m}Tc chelators linked to D-Glu₂₀ (DE20) and D-Glu₁₀ (DE10) were synthesized via standard Fmoc solid-phase peptide synthesis as described previously. Wang resin loaded with Fmoc cysteine (TRT) was coupled to Fmoc aspartic acid (OtBu) then to N^{α}-Boc-N^{β}-Fmoc-L-2,3-diaminopropionic acid to create the ^{99m}Tc chelator (see, e.g., Leamon et al., “Synthesis and biological evaluation of EC20: A new folate-derived, 99mTc-based radiopharmaceutical,” Bioconjug. Chem. 13: 1200-1210 (2002)). This chelator was then coupled via standard amide chemistry to 8-(Fmoc-amino)-3,6-dioxaoctanoic acid, which was then conjugated via standard amide coupling to a linear oligopeptide of either 10 or 20 D-glutamic acids. The oligopeptide was then cleaved and purified as described previously.

### Example 8: Synthesis of Near Infrared (NIR) Dye Conjugates

A maleimide derivative of the near-infrared (NIR) fluorescent dye, 50456, was prepared for use in labeling of the bone fracture targeting ligands described above. It was synthesized as shown in Scheme I (below). For this purpose, S0456, N-Boc-tyramine and potassium hydroxide (KOH) were mixed in a flask containing dimethylsulfoxide (DMSO) to dissolve solids and the solution was stirred at 60° C. under argon for 1.2 hours. The resulting solution was precipitated with cold ethyl acetate and, after vigorous agitation, was centrifuged at 3,000 rpm for 3 minutes. The dark green solid was dried in a vacuum desiccator overnight and deprotected in 40% trifluoroacetic acid (TFA)/DCM for 30 minutes before being concentrated in vacuo to remove all TFA and DCM. The crude solid was then dissolved in water and subjected to preparative reversed-phase high-performance liquid chromatography (1290, Agilent, Santa Clara, CA) purification. Pure fractions were concentrated in vacuo and lyophilized. To derivatize with maleimide, the solid was dissolved in DMSO together with N-succinimidyl 3-maleimidopropionate and DIPEA and stirred under argon atmosphere for one hour before purification via preparative reversed-phase high-performance liquid chromatography (1290, Agilent, Santa Clara, CA) as described above. Deca-aspartic acid (L and D)-targeting ligand with an N-terminal cysteine were prepared and purified as described previously. For conjugation of deca-aspartic acid cysteine to S0456-maleimide, S0456-maleimide was dissolved in DMSO in a flask degassed with argon, followed by the addition of Asp₁₀-Cys to the solution with stirring. The mixture was stirred at room temperature for 2.5 hours before purification with preparative reversed-phase high-performance liquid chromatography (1290, Agilent, Santa Clara, CA). The purified and lyophilized product appeared as a green fluffy solid. Synthesis of (D)Asp₁₀-S0456 conjugate followed the same procedure as described for (L)Asp₁₀-S0456, except that D-aspartic acid was used for the synthesis of (D)Asp₁₀.

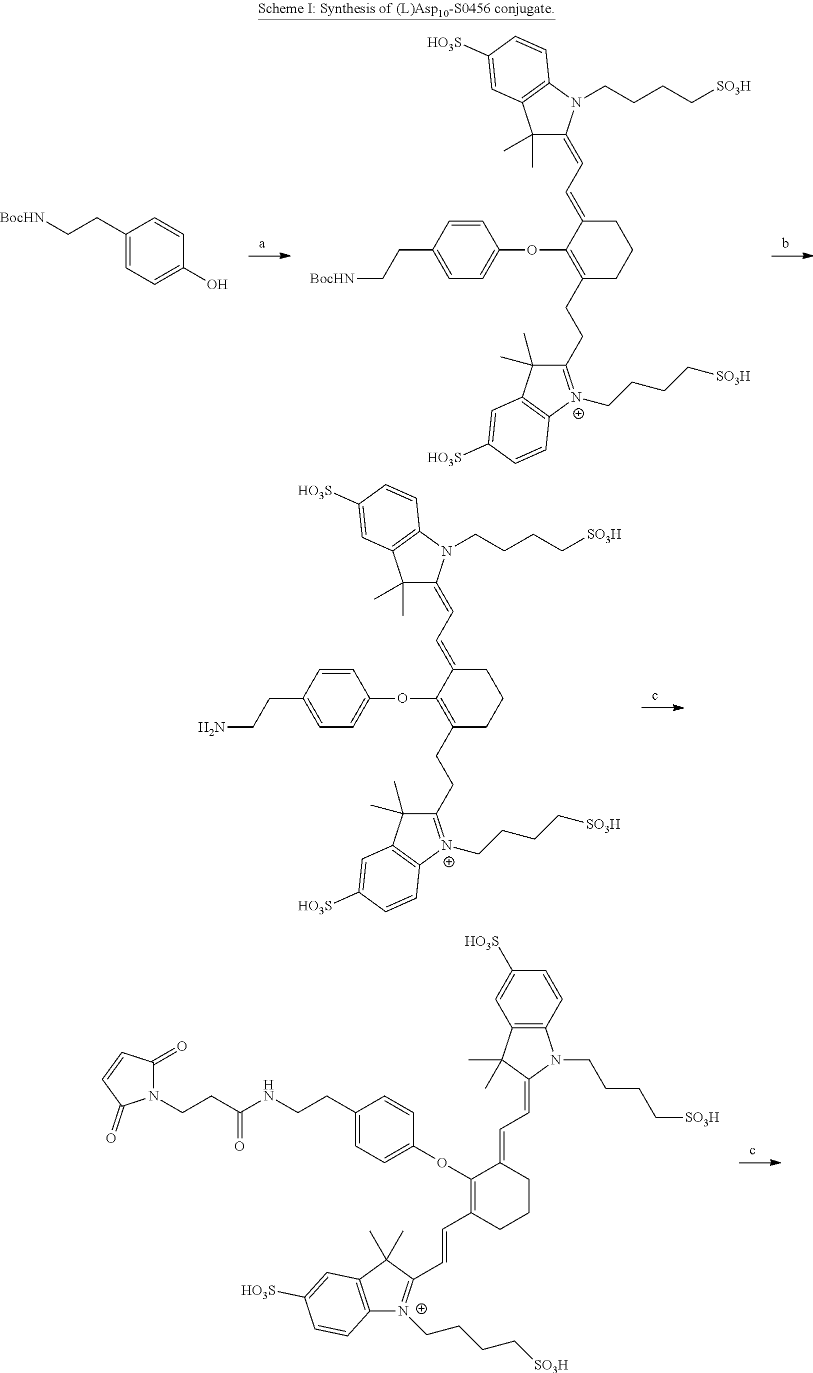 [SMILES: CC(C)(C)OC(=O)NCCc1ccc(O)cc1 ; CC(C)(C)OC(=O)NCCc1ccc(OC2=C(CCC3=[N+](CCCCS(=O)(=O)O)c4ccc(S(=O)(=O)O)cc4C3(C)C)CCC/C2=C\C=C2\N(CCCCS(=O)(=O)O)c3ccc(S(=O)(=O)O)cc3C2(C)C)cc1 ; CC1(C)C(CCC2=C(Oc3ccc(CCN)cc3)/C(=C/C=C3/N(CCCCS(=O)(=O)O)c4ccc(S(=O)(=O)O)cc4C3(C)C)CCC2)=[N+](CCCCS(=O)(=O)O)c2ccc(S(=O)(=O)O)cc21 ; CC1(C)C(/C=C/C2=C(Oc3ccc(CCNC(=O)CCN4C(=O)C=CC4=O)cc3)C(=C/C=C3/N(CCCCS(=O)(=O)O)c4ccc(S(=O)(=O)O)cc4C3(C)C)/CCC2)=[N+](CCCCS(=O)(=O)O)c2ccc(S(=O)(=O)O)cc21]

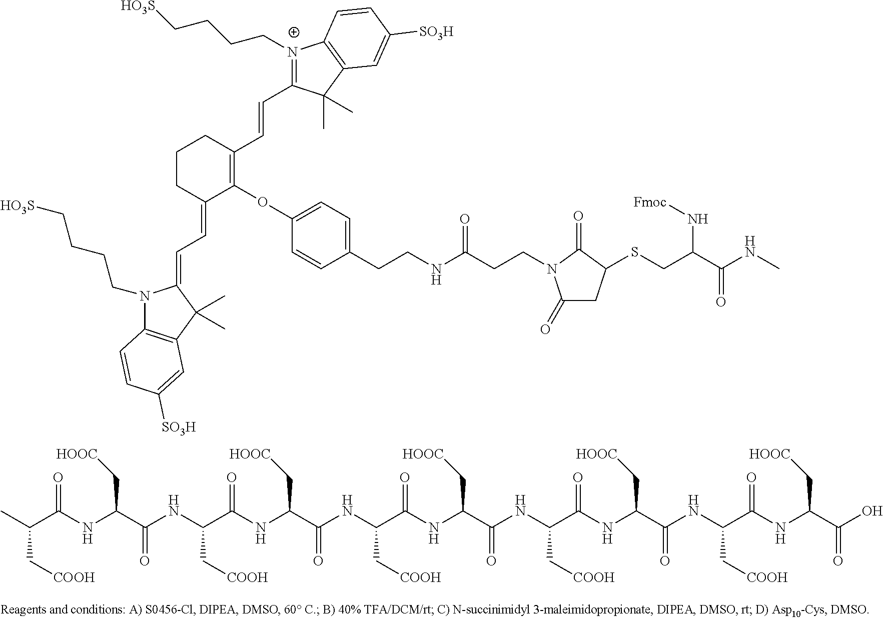

### Example 9: Midshaft Femur Fracture Model

Aseptic surgical techniques were used to insert a 23-gauge needle as an intramedullary nail into the femur of anesthetized 12-week-old female ND-4 Swiss-Webster age-matched mice for internal fixation on the bone prior to its fracture. No difference in targeting capacity was seen between inbred strains such as C57/BL6 and Swiss-Webster ND-4 mice. Briefly, the mouse hair surrounding the right knee of the hind paw was removed and the animal was anesthetized using 3% isoflurane with an anesthesia vaporizer (VetEqip, Livermore, CA). The skin was then cleaned with a scrub of betadine followed by a scrub of 70% ethanol. An incision was then made over the patella, exposing the patellar tendon, and the tendon was transected to expose the distal condyles of the femur. A sterile 23-gauge needle was drilled through the cortical shell of the center of the patellar surface at the distal femur between the condyles. A pin was inserted down the center of the medullary cavity until it reached the endosteal surface of the proximal epiphysis of the femur. The needle was then cut with wire cutters to render it flush with the distal end of the femur, and the skin was closed with 4-0 nonabsorbable nylon sutures. Fractures were then induced in the stabilized femurs using a drop-weight fracture device from RISystem and were verified via X-ray using an X-ray cabinet (Carestream, Kodak, Rochester, NY). The mice received buprenorphine (0.03 mg/day) for three days post-fracture to reduce pain. All animal experiments were performed in accordance with protocols approved by Purdue University's Institutional Animal Care and Use Committee (IACUC).

### Example 10: Half-Life and Biodistribution

To analyze the half-life of the targeted fluorescent conjugates at the fracture site, L-Asp₁₀-S0456 or D-Asp₁₀-S0456 was dissolved in PBS, sterile filtered, and injected 10 days post-fracture subcutaneously to achieve a final dose of 250 nmol/mouse. Mice were then euthanized at 2, 24, 48, 72, and 96 hours post-injection, and fluorescence was quantified at the fracture site by resecting and dissolving the fracture callus in a 12% solution of neutral buffered ethylenediaminetetraacetic acid (EDTA). Briefly, broken femurs were collected, rinsed with PBS, dried thoroughly overnight in a vacuum desiccator, broken into small pieces, and weighed before immersing in the aforementioned EDTA solution. The sample was agitated on a shaker for 8 hours to decalcify the bone and then centrifuged for 5 minutes at 8,000 rpm to collect the supernatant. The concentration of L-Asp₁₀-S0456 or DAsp₁₀-S0456 in the supernatant was then determined from its OD780 using a standard curve of known concentrations of the dye for quantitation.

### Example 11: Radiolabeling of Peptides

Ten (10) μg of Pierce Iodination (iodogen; Thermo Fisher Scientific, Waltham, MA) reagent were dissolved in 200 μL chloroform, then added to a 6×50 mm glass test tube and evaporated under a steady stream of argon. Then 50 nmol of peptide conjugate dissolved in 40 μL of PBS were added together with 10 μL (1 mCi) of Na¹²⁵I(ARC). The glass test tubes were sealed and placed on a shaker for 30 minutes and then purified via radio preparative reversed-phase high-performance liquid chromatography (1260 HPLC; Agilent Flow-RAM radiodectector, Lablogic Systems Ltd, Sheffield, UK) with a 0-100% gradient of 0.1% TFA in water:acetonitrile. Fractions with the correct retention time and radio signal were isolated and lyophilized. Payload peptides were radio-iodinated on endogenous tyrosine, tryptophan, or histidine residues, which remain stable in physiological conditions for the longest iodinated experiments (27 hours) (see Savoie et al., “Studies on mono- and diiodohistidine. I. The identification of iodohistidines from thyroidal iodoproteins and their peripheral metabolism in the normal man and rat,” J. Clin. Invest. 52: 106-115 (1973)).

For ^{99m}Tc labeling, 0.6 mg of EDTA disodium dihydrate dissolved in nitrogen-sparged water (10 mg/ml) was added to 50 mg of sodium gluconate solution (100 mg/ml) in nitrogen-sparged water. To that mixture, a solution of 0.2 mg of tin chloride dihydrate (10 mg/ml) dissolved in nitrogen-sparged 0.2N HCl was added. Then 4 μmol of ^{99m}Tc chelate-containing peptides were added to the solution, and the pH was adjusted to 6.8 using NaOH (Leamon et al. (2002), supra). The solution was flash-frozen in liquid nitrogen and lyophilized overnight. The compound was then mixed with 15 mCi of m⁹⁹Tc (Cardinal Health) and after 15 minutes of shaking, quantitative chelation was confirmed by analytical radio reversed-phase high-performance liquid chromatography (1260 HPLC; Agilent Flow-RAM radiodectector, Lablogic).

### Example 12: Biodistribution Analysis

For live animal studies, ^{99m}Tc- or ¹²⁵I-radiolabeled peptides were dissolved in PBS and injected subcutaneously into mice 10 days after induction of a midshaft femur fracture to ensure that blood flow had returned to the area. Each mouse received a 0.25 mCi (12.5 nmol of peptide in 0.1 mL vehicle) dose of radio-iodinated peptide or 3 mCi (0.1 ml) dose of ^{99m}Tc-labeled peptide, both administered subcutaneously. Eighteen hours later, blood was removed via cardiac puncture, and mice were sacrificed via CO₂ asphyxiation. Organs and tissues (heart, lungs, muscle, skin, liver, spleen, kidneys, fractured femur, and healthy femur) were resected and weighed, and their radioactivity was counted using a gamma counter (Cobra Auto-Gamma, Packard; GMI Corporation, Franklin, IN). Percent injected dose was calculated by:

$$\% injected dose = \frac{Tissue ( counts )}{Injection ( counts ) \times Tissue ( grams )} \times 100$$

Fractured to healthy ratio was calculated by:

$$Fractured to healthy ratio = \frac{The fractured {femur}^{′} s \% injected dose}{The healthy {femur}^{′} s \% injected dose}$$

### Example 13: SPECT/CT

^{99m}Tc labeled D-Glu₁₀-chelator and D-Glu₂₀-chelator were formulated to 7 mCi/100 μl and injected via tail vein two weeks following femur fracture. After 18 hours, mice were euthanized via CO₂ asphyxiation and imaged using a single-photon emission computer tomography/computed tomography (SPEC/CT) scanner (U-SPECT-II/CT, MiLabs, Houten, The Netherlands). CT images were collected using high-resolution, full-body, 12-minute scans and were followed by 1-hour SPECT scans using a 0.6 mm collimator. SPEC/CT images were reconstructed using the MiLabs software selecting the energy window of 140 keV and reconstruction parameters of 16 subsets and 4 iterations without post filter. 3-D reconstructions were performed using ImageJ software.

### Example 14: Fracture Repair Model—Diabetes

Targeted conjugates of SEQ ID NO: 5, abaloparatide (SEQ ID NO: 2), SEQ ID NO: 6, and SEQ ID NO: 9 (a conjugate with 10 aspartic acid residues) were synthesized using Fmoc solid-phase peptide synthesis. From SEQ ID NO: 5, amino acids 1-34 were used, and abaloparatide (SEQ ID NO: 2) is a stabilized version of amino acids 1-36 of SEQ ID NO: 1. Diabetes was induced in 40 8-week-old male CD-1 mice via seven subcutaneous injections of streptozotocin (STZ) until blood sugar readings were above 250 mg/dL. The mice were left in this confirmed diabetic state for 2 months to allow the diabetes to take effect on the bones. Then, aseptic surgical techniques were used to place a 23-gauge needle as an intramedullary nail in the femur of the anesthetized mice for internal fixation before fracture was induced with a drop-weight fracture device from RISystem and confirmed by x-ray. The mice received buprenorphine for three days post-fracture. They were dosed subcutaneously with a positive control of insulin, a negative control of vehicle, SEQ ID NO: 5, or abaloparatide (SEQ ID NO: 2) each day for 4 weeks. Fracture healing was assessed qualitatively using micro-CT (Scanco Medical Ag). Fractured femurs were tested for strength in a 4-point-bend-to-failure using an ElectroForce TestBench (TA Instruments, New Castle, DE). Maximum load, stiffness, displacement post-yield, and work-to-fracture data were generated. Statistical analysis was performed using a one-way analysis of variance (ANOVA) with significance reported with p values less than 0.05 (*) and 0.01 (**). All animal experiments were performed in accordance with protocols approved by Purdue University's IACUC.

Four compounds were tested. Abaloparatide(D)_e10, SEQ ID NO: 6, and SEQ ID NO: 7 (FIG. 1A and FIG. 1B) had all repeatedly accelerated healing in healthy femur fractures. Abaloparatide (SEQ ID NO: 2) is a stabilized version of parathyroid-related protein hormone. Dasatinib is an SRC kinase with off-target effects on both osteoblasts and osteoclasts that improve overall bone density. Dasatinib has also proven to be a senolytic. ITGA is a fibronectin mimetic that promotes intramembranous bone fracture healing. SEQ ID NO: 5 was included in this study because Preptin 1-16 had a moderate bone anabolic activity. It was also shown that the full-length compound (see FIG. 1A and FIG. 1B) also improved glucose sensitivity. Therefore, it was hypothesized that the glucose regulating properties of full-length SEQ ID NO: 5 may be beneficial in healing type I diabetic fractures as a dual-action compound that may improve healing via two mechanisms. The compounds were compared against insulin as a positive control and saline as a negative control.

All targeted compounds except for SEQ ID NO: 6 improved the mineralization and bone density relative to saline, with SEQ ID NO: 5 impacting callus mineralization the most and abaloparatide impacting density the most. The results are shown in FIGS. 13 and 14. BV represents the bone volume of the 100 thickest micro-CT slices of the fracture callus and is a measure of how much bone has mineralized at the site of fracture repair. BV/TV represents the BV divided by TV of the 100 thickest micro-CT slides of the fracture callus and is a measure of how dense the bone is at the site of fracture repair. Insulin was dosed as 2 IU/day. Doses of 0.1×, 50×, and 10×, are nmol, 50 nmol, and 10 nmol, respectively, of the conjugate delivered daily by subcutaneous injection. SEQ ID NO: 6 was dosed at 10 μmol/kg every other day.

All targeted compounds improved the strength of the fractures more than just insulin treatment alone. Despite insulin's improvement in mineralization, it was not able to restore the quality of the bone that was damaged by hyperglycemia, so the bones were still weak. Bone brittleness is what leads to so many fractures in diabetics. A measure of brittleness is post-yield displacement in a 4-point-bend test. The results are shown in FIGS. 15 and 16.

Insulin was dosed at 2 IU/day. Doses of 0.1×, 50×, and 10×, are 0.1 nmol, 50 nmol, and nmol, respectively, of the conjugate delivered daily by subcutaneous injection. SEQ ID NO: 6 was dosed at 10 μmol/kg every other day.

All compounds except for SEQ ID NO: 6 reduced the brittleness of the bone (FIG. 17). Insulin was the only compound to improve blood sugar levels (FIG. 18). By treating the bone fractures, the animals were able to regain the weight that they were losing because of diabetes (FIG. 19).

However, since non-diabetic patients will not stop taking their insulin to take a compound provided herein, the experiment was repeated with compounds dosed in conjunction with insulin (FIGS. 20-22).

For FIGS. 20A and 20B, insulin was dosed at 2 IU/day and doses of 0.1×, 10×, and 100×, are 0.1 nmol, 10 nmol, and 100 nmol, respectively, of the conjugate delivered daily by subcutaneous injection. The figures show that SEQ ID NO: 8 improved the mineralization of the callus better than insulin, and all the experimental therapeutics led to increases in fracture callus density, more so than insulin alone.

For FIGS. 21A and 21B, insulin was dosed at 2 IU/day and doses of 0.1×, 10×, and 100×, are 0.1 nmol, 10 nmol, and 100 nmol, respectively, of the conjugate delivered daily by subcutaneous injection. The figures show no significant changes were observed in the trabecular bone.

For FIGS. 22A-22C, insulin was dosed at 2 IU/day in all groups, except for saline. Doses of 0.1×, 10×, and 100×, are 0.1 nmol, 10 nmol, and 100 nmol, respectively, of the conjugate delivered daily by subcutaneous injection. The figures show that abaloparatide (SEQ ID NO: 2) and SEQ ID NO: 8 significantly improved the strength compared to the insulin control. All significance levels were calculated relative to the insulin-treated group, not the saline control group. Overall, SEQ ID NO: 9 did not significantly improve fracture healing in diabetics. However, abaloparatide (SEQ ID NO: 2) and SEQ ID NO: 8 improved the strength and mineralization more than insulin alone, and they show promise as potential therapeutics.

### Example 15: Fracture Repair Model—Post-Menopausal Osteoporosis

The increased resorption pits present throughout the skeleton during osteoporosis serve as moderate localization sites in the presence of osteoporosis alone. However, localization does still occur elsewhere in the skeleton during osteoporosis. The majority still localizes to the fracture site. This could lead to some populations needing to be contraindicated in certain types of bone-targeted therapies. However, for osteoporosis patients with a fracture, the off-target skeletal effects to heal the resorption pits would actually be a positive side effect and might not present a problem. It would be a two-fold effect in which the fracture is healed and the osteoporosis is treated as well to prevent future fractures.

Eight-week-old female Swiss Webster mice (n=10) were surgically induced with post-menopausal osteoporosis via bilateral ovariectomy. Osteoporosis was confirmed via a lost in overall bone mineral density as measured by microCT.

Mice were anesthetized using 2-3% isoflurane. Buprenorphine (0.03 mg/kg) was administered subcutaneously for postoperative pain relief. A 3 cm×2.5 cm area was shaved dorsally from the iliac crest. The area was washed with betadine followed by 70% ethanol and then draped. A 2-cm midline incision was made, and the skin was dissected from the underlying fascia. A 1-cm lateral incision of the midline was made through the fascia, reaching the abdominal cavity. The adipose tissue surrounding the ovary in the abdominal cavity was pulled back and gently pulled out.

The ovary was isolated and the uterine horns and vessels 0.5 cm proximally of this structure were ligated. The ovary was removed, and the process was repeated on the contralateral side. The peritoneal cavity was closed, followed by the skin using a monofilament suture. The mouse was placed in a clean recovery cage and allowed to awake from anesthesia. Mice were dosed every 12 hours with buprenorphine for 3-5 days. Osteoporosis was expected to develop within 4-6 weeks, at which point the mice underwent a stabilized femoral fracture, as described above.

Bone mineral density (BMD) was measured before ovariectomy and eight weeks after ovariectomy to confirm development of osteoporosis. After 8 weeks of prolonged exposure to low estrogen levels, the mice had intermedullary nails placed in their femurs and then an Einhom fracture model was induced via a drop weight and confirmed by X-ray. The mice were dosed daily for 4 weeks. The structural changes were quantified via micro-CT, and the mechanical properties were assessed via a 4-point-bend-to-failure test.

S0456, boc-tyramine, and KOH were charged into a degassed 50-mL RB flask. DMSO (5 mL) was added to dissolve all solid, and then the mixture was stirred at 60° C. under argon atmosphere for 1.2 hours. The resulting mixture was cooled to room temperature and added to cold ethyl acetate (50 mL) dropwise. The resulting mixture was vigorously agitated, then centrifuged at 3,000 rpm for 3 minutes. After centrifugation, dark green solid could be seen on the bottom of the conical tube. The supernatant (very slightly greenish clear solution) was discarded before a new batch of cold ethyl acetate was added to the solid. The mixture was vigorously agitated and subjected to the same centrifugation process as before. The resulting dark green solid was dried in a vacuum desiccator overnight before 40% TFA/DCM solution (5 mL) was added. The mixture was stirred at room temperature for 30 minutes before being concentrated in vacuo to remove all TFA and DCM. The concentrated crude was dissolved in 3 mL water and subjected to prep-HPLC purification. Pure fractions of S0456-tyramine were collected, concentrated in vacuo, frozen, and lyophilized to yield the pure product as a fluffy, dark green solid. This solid (120 mg) was dissolved in DMSO (3 mL) together with N-succinimidyl 3-maleimidopropionate (30 mg) and DIPEA (40 μL) and stirred under argon atmosphere for 1 hr before purification with prep-HPLC. Pure fractions of 50456-maleimide were collected, concentrated in vacuo, frozen, and lyophilized to yield the pure product as a dark green fluffy solid (125 mg).

D₁₀-Cys was synthesized as follows. For the initial loading of the SPPS resin, 2-chlorotrityl chloride resin (0.4 g, 1.4 mmol/g) was swollen in DCM (10 mL/g resin) followed by the addition of Fmoc-L-Asp(OtBu)-OH (1.15 g, 2.8 mmol) and DIPEA (1.66 mL, 9.5 mmol) dissolved in DCM (14 mL). The mixture was agitated by bubbling argon for 1 hr, after which the solution was drained before 20 mL of capping cocktail (DCM:MeOH:DIPEA=17:2:1) were added and the solution was bubbled again for 20 minutes. The resin was then subjected to standard washing procedures, which consisted of washes with DMF (3 times), DCM (3 times), and IPA (3 times) following each coupling reaction, and washes with DMF (3 times) following each deprotection. After the initial loading, all subsequent coupling reactions were performed with solutions of Fmoc-L-Asp(OtBu)-OH (1.15 g, 2.8 mmol) or Fmoc-S-trityl-L-cysteine (1.64 g, 2.8 mmol), PyBOP (1.42 g, 2.75 mmol), and DIPEA (1.66 mL, 9.5 mmol) in DMF (14 mL). One-hour standard coupling time was used for all aspartic acid and cysteine residues. Fmoc-deprotection was done with 20% piperidine solution in DMF for two sessions of 5 minutes and 10 minutes each. The 11-mer peptidic product was cleaved off the resin using a cleavage cocktail consisting of 90% TFA, 3.3% TIPS, 3.3% water, and 3.3% EDT. Following cleavage, the crude product was concentrated under reduced pressure to remove most TFA, water, TIPS, and EDT, and then washed 3× with Et₂O and dried under reduced pressure for 24 hours to produce D₁₀-Cys 1 as a white powder (680 mg, 81.3% overall yield, 98.1% average coupling efficiency).

D₁₀-S0456 conjugate was synthesized as follows. S0456-maleimide (100 mg) was dissolved in 2 mL DMSO in a flask degassed with argon, followed by the addition of D₁₀-Cys to the solution with stirring. The mixture was stirred at room temperature for 2.5 hours before purification with prep-HPLC. The purified and lyophilized product appeared as a green fluffy solid (57 mg).

Female 12-week-old Swiss Webster that had undergone midshaft femur fractures and a bilateral ovariectomy as described previously were injected subcutaneously near the nape with 10 nmol of S0456 conjugate. After 24 hours they were sacrificed, and a necropsy was performed. The tissues were imaged in a Spectral Ami Optical Imaging System with an excitation for 1 second at 745 nm at 5% excitation power. The fluorescent emission was collected at 810 nm.

For quantitative determination of calcium ion (Ca²⁺) and evaluation of drug effects on calcium metabolism, the QuantiChrom Calcium Assay Kit from BioAssay Systems (Hayward, CA) was used. A phenolsulfonphthalein dye in the kit specifically forms a very stable, blue-colored complex with free calcium. The intensity of the color, measured at 612 nm, is directly proportional to the calcium concentration in the sample. The optimized formulation minimizes any interference by substances such as magnesium, lipid, protein, and bilirubin. One mL of blood was collected from the mice at the end of the therapeutic study after 21 days of dosing via cardiopuncture under anesthesia induced by 3% isoflurane. The blood was spun at 500 G for 5 minute to pellet the cells. The serum was removed from the cell pellet and stored at −80° C. until calcium concentration quantification. The standard was diluted to 10 mg/dL Ca²⁺ by mixing 125 μL 20 mg/dL standard and 125 μL dH₂O. A whole blood sample (5 μL) was transferred to a well. Next, 200 μL of working reagent were added, and the plate was tapped lightly to mix. After that, the samples were incubated for 3 minute at room temperature and optical density at 570-650 nm (peak absorbance at 612 nm) was read to obtain the ODSAMPLE. After that, 10 mg/dL standard (5 μL) was transferred to the sample well. The plate was tapped to mix and optical density was measured at the same wavelength to get ODSTANDARD. Next, 5 μL of 20 mM EDTA were added to the same well from earlier and the plate was tapped to mix. Optical density was read at the same wavelength measurement to get ODBLANK. The whole blood sample concentration was computed as follows:

[Ca2+]=(ODSAMPLE−ODBLANK)/(ODSTANDARD−ODSAMPLE)×10×n (mg/dL)

where ODSAMPLE, ODBLANK, and ODSTANDARD are the OD readings of the Sample, Sample Blank, and the Sample plus Standard respectively; 10 is the concentration of the standard in mg/dL, and n is the sample dilution factor. If the calculated calcium concentration was greater than 10 mg/dL, the sample was diluted in dH₂O and the assay was repeated. We then multiplied the result by the dilution factor n.

Initially, four compounds were tested. SEQ ID NO: 3, SEQ ID NO: 6, and SEQ ID NO: 7 had all repeatedly accelerated healing in healthy femur fractures, with abaloparatide (SEQ ID NO: 2) being a stabilized version of parathyroid-related protein hormone. Dasatinib is an SRC kinase with off-target effects on both osteoblasts and osteoclasts that improve overall bone density. It has also been shown to be a senolytic. ITGA is a fibronectin mimetic that promotes intramembranous bone fracture healing.

All targeted compounds, except for SEQ ID NO: 10 (a conjugate with 20 glutamic acid residues), improved the mineralization and density of the fracture callus relative to saline and more so than estrogen replacement (FIGS. 23A-B).

For FIGS. 23A and 23B, estrogen benzoate was dosed at 30 μg/kg weekly. Doses of 0.1×, 1×, and 10× are 0.1 nmol, 1 nmol, and 10 nmol, respectively, of the conjugate delivered daily by subcutaneous injection. SEQ ID NO: 6 was dosed at 10 μmol/kg every other day.

The same three compounds proved to be effective in improving strength of the femurs (FIGS. 24A-C).

With regard to FIGS. 24A-C, estrogen benzoate was dosed at 30 μg/kg weekly. The doses 0.1×, 1×, and 10× are 0.1 nmol, 1 nmol, and 10 nmol of the conjugate, respectively, delivered daily by subcutaneous injection. SEQ ID NO: 6 was dosed at 10 μmol/kg every other day.

Many of the compounds significantly improved the healing of fracture femurs in this hypoestrogenic state far better than controlling the loss of estrogen with estrogen replacement. This could make targeted compounds attractive for helping osteoporotic patients with bone fractures heal faster. As noticed with diabetic mice, all metrics of callus healing in the control mice were significantly lower than those of standard healthy mice, representing a significantly challenged population of patients.

The effects of treating osteoporotic mice with either free anabolic (SEQ ID NO: 1) or targeted anabolic (abaloparatide_D₁₀ (a targeted stabilized version of SEQ ID NO: 1)) for four weeks were examined. Twenty-month-old Swiss Webster female mice that had developed age-induced-osteoporosis were examined, since older mice naturally develop reduced bone density. The average bone mineral density present in the femurs, pelvis, and lumbar vertebra—the regions most effected by osteoporosis—were quantified via PerkinElmer micro-CT using a standard phantom to quantify the bone mineral density of the affected portion of the skeleton. Mice were treated for 4 weeks.

Targeted and free therapies improved BMD of the treated mice. Other metrics will need to be evaluated to determine the benefit of targeted drugs in the treatment of osteoporosis by itself. If both are valid in improving, the targeted drug could still be desirable in terms of reducing side effects. The free osteoporotic drugs are limited by their systemic side effects. The parathyroid family in particular is limited by its effects on blood calcium. But as evidenced in FIG. 25, the targeted form of Forteo® (SEQ ID NO: 13) did not increase the blood calcium significantly in comparison to the free form.

Free PTH (targeted form of Forte®) at 0.1 nmol was compared to targeted PTH (SEQ ID NO: 13) using saline as control. So even though osteoporosis is a systemic disease, there are advantages to using targeted anabolics as opposed to free anabolics, because targeted anabolics can limit the effects on the regulation of calcium metabolism that occurs in the kidneys for parathyroid hormones.

### Example 16: Fracture Repair Model—Maxillofacial

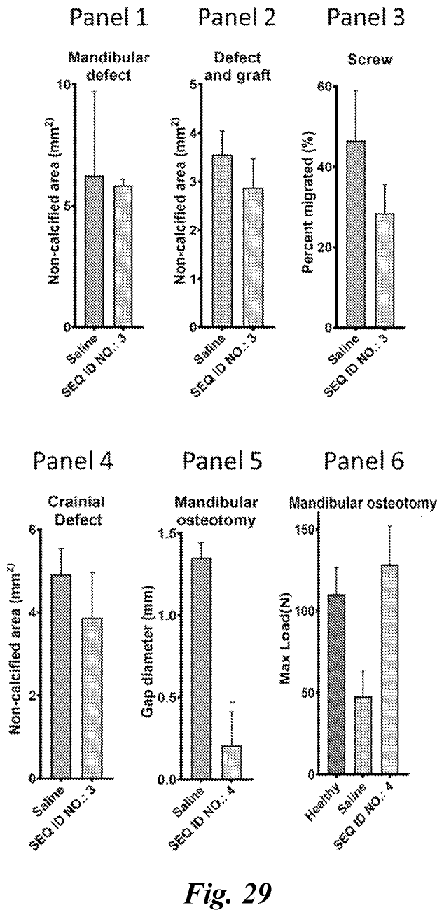

The results of the application of the compounds were examined in the following five rat models: (i) jaw fractures, modeled using a non-critical size mandibular defect (FIG. 29, panel 1), (ii) major maxillofacial surgeries, modeled by introducing a critically sized defect filled with bone graft material (FIG. 29, panel 2), (iii) osseo-integration of implanted metals (FIG. 29, panel 3), (iv) a cranial critically sized defect (FIG. 29, panel 4), and (v) a microplate stabilized mandibular osteotomy (FIGS. 29, panel 5 and 6). Some degree of healing was observed for all the surgeries, except for the mandibular defect. The mandibular osteotomy, stabilized with a microplate, evidences the greatest potential for future success. By three weeks post-surgery, dramatic improvements were observed in the reduction of the osteotomy gap, where the diameter of the treated group's gap was 13% of the diameter of the saline control (p<0.01). The microarchitecture was also improved. In the treated group, the structure of the bone had remodeled to nearly normal bone, whereas an obvious gap was still present in the saline group. Finally, the maximum load that the treated jaws could withstand prior to failure was 2.7-fold that of saline (p<0.5) and, when the strength of the treated jaws was compared to the strength of the contralateral jaw, it was found that they had returned to the strength of the non-broken bone. The results are summarized in FIG. 29, which shows graphs of agent vs. non-calcified area (mm 2) for defect and graft, and cranial defect, agent vs. percent migrated (%) for screw, agent vs. gap diameter (mm) for mandibular osteotomy, and agent vs. max load (N) for mandibular osteotomy.

### Example 17: Pain/Function Outcomes

Pain/function outcomes in mice were accessed by turning to behavioral testing. Allodynia was evaluated using Von Frey probes, whereas gain was evaluated using DigiGait, functionality was evaluated using running wheels, and anxiety coupled with functionality was evaluated using locomotor open-field testing. Open-field locomotor testing afforded the most consistent measurements.

Mice were habituated to the behavior room for 30-60 minutes before testing locomotor activity. Animals were habituated once for 10 minutes to the locomotor boxes prior to the start of the experiment. They were then placed individually in a locomotor box with infrared light tracking beams for 10 minutes before being removed and placed back in their home cage. The mice were tracked via EthoVision using 3-point directionality testing. Mice were measured for two weeks prior to the experiment. They then underwent the midshaft femur fracture model and were assigned to one of three treatment groups: 1) abaloparatide DE20 twice a week; 2) phosphate buffered saline twice a week; or 3) ibuprofen (0.6 g/L) in their water. The mice were treated for 5 weeks post-fracture and measured once a week.

In their baseline state, all mice ran everywhere in their box—an indication that they felt fine. However, after the fracture, the mice treated with saline and ibuprofen preferred to stay on the edge and in the corner, which indicated that they were feeling unwell and anxious. The abaloparatide-treated mice returned to their previous state of well-being and ran everywhere. It is interesting that the ibuprofen mice did not improve their pain/function outcomes. Ibuprofen is known to inhibit bone fracture repair, so the analgesia received might be overcome by the decreased healing of the animal. However, the accelerated healing of the fracture by the SEQ ID NO: 4 mice did lead to more functionality and better well-being. Not only did the mice localize to regions that indicate they had better well-being, but they also quantitatively improved their functionality metrics in the locomotor portion of this test as shown in FIGS. 26-28.

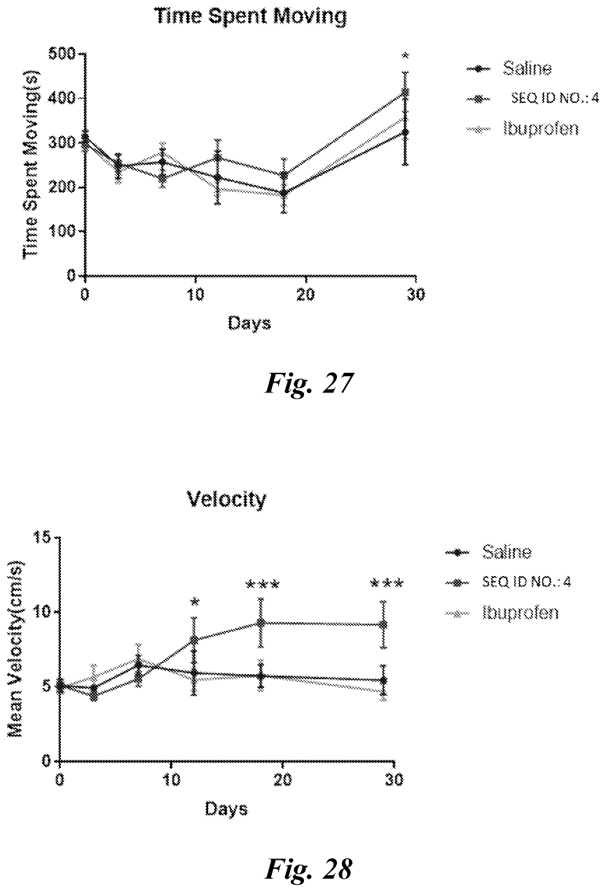

FIG. 26 is a graph of days vs. distance traveled (cm), which shows the average total distance traveled in a 10-minute period of time in locomotor open-field boxes for the different treatment groups (n=7). FIG. 27 is a graph of days vs. time spent moving, which shows the average time spent moving in a 10-minute period of time in locomotor open-field boxes for the different treatment groups (n=7). FIG. 28 is a graph of days vs. mean velocity (cm/s), which shows the average velocity in a 10-minute period of time in locomotor open-field boxes for the different treatment groups (n=7).

For FIGS. 26-28, day 0 represents the baseline data pre-fracture. Saline and SEQ ID NO: 4 (1 nmol) were injected twice/week. Ibuprofen was administered constantly in the mice's water at a concentration of 0.6 g/L.

FIG. 26 shows that the abaloparatide-treated mice began to run farther, whereas FIG. 27 shows that they began to run for longer periods of time and FIG. 28 shows that the abaloparatide-treated mice began to run faster. All these metrics indicate increased functionality and better performance, which indicates that the increase in structural and mechanical healing quantified via micro-CT and biomechanical testing also corresponds to improved functionality and reduced pain. These results exceed what reportedly has been seen with BMP treatment, i.e., that BMP does not improve pain or functionality, despite being approved for bone fracture repair. So, by dosing an animal with SEQ ID NO: 4, a targeted bone anabolic agent that returns bones to their previous unbroken strength between two and three weeks post-fracture, a reduction in pain can be realized by that time.

### Example 18: PK Properties

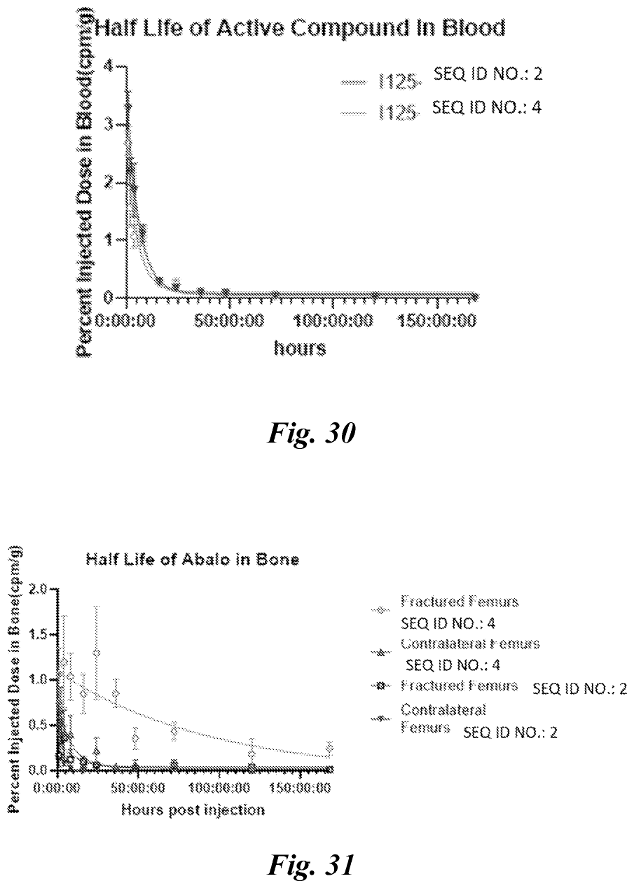

SEQ ID NO: 4 was subcutaneously injected into fracture-bearing mice. No significant differences were observed in either the circulation half-life (4.2 hours vs. 3.7 hours) or cumulative systemic exposure (AUC; 24.4 hours and 20 hours, respectively) of ¹²⁵I-labeled abaloparatide and SEQ ID NO: 4, indicating that any off-target exposure or resulting systemic toxicity should be similar between targeted and non-targeted drugs. The results are shown in FIG. 30, which is a graph of hours vs. percent injected dose in blood (cpm/g) of mice with midshaft femoral factures. In contrast, a major difference was observed in the residence times of the two abaloparatides at the fracture site, where the half-life of SEQ ID NO: 4 was found to be 67 hours, while that of non-targeted abaloparatide (SEQ ID NO: 2) was 8.8 hours. The difference in half-life resulted in an 11-fold larger cumulative residence time (AUC) of SEQ ID NO: 4 relative to abaloparatide (SEQ ID NO: 2) (i.e., 96 hours compared to 8 hours). The residence time of SEQ ID NO: 4 in the contralateral femur was 8-fold lower than its residence time in the fractured femur, indicating that concerns regarding stimulation of growth of healthy bones should be minimal. The results are shown in FIG. 31, which is a graph of hours vs. percent injected dose in bone (cpm/g) of mice with midshaft femoral factures. Thus, although the systemic exposures of D-Glu₂₀-targeted and non-targeted abaloparatides are similar, the significantly enhanced concentration of SEQ ID NO: 4 at the fracture surface is expected to improve the repair rate of the associated fracture.

It is recognized that various modifications are possible within the scope of the claimed invention. Thus, it should be understood that, although the present invention has been specifically disclosed in the context of preferred embodiments and optional features, those skilled in the art may resort to modifications and variations of the concepts disclosed herein. Such modifications and variations are considered to be within the scope of the invention as claimed herein.

1. A compound having a structure of Formula (I)
X—Y—Z Formula (I),
or a pharmaceutically acceptable salt thereof,
wherein:
X is a bone anabolic agent selected from the group consisting of a parathyroid hormone (PTH) or a derivative or fragment thereof, a PTH-related protein (PTHrP) or a derivative or fragment thereof, and abaloparatide or a derivative or fragment thereof;
Y is absent, a releasable linker or a non-releasable linker; and
Z is an osteotropic ligand.

2. The compound of claim 1, wherein X is a an abaloparatide or a derivative or fragment thereof comprising SEQ ID NO: 3, wherein x is methylalanine and “e” indicates D-chirality.

3. The compound of claim 1, wherein the osteotropic ligand of Z is an acidic oligopeptide (AOP) comprising at least 11 amino acid residues.

4. The compound of claim 3, wherein the AOP comprises 11 to 100 amino acid residues.

5. The compound of claim 1, wherein X is a bone anabolic agent selected from the group consisting of a PTH or a derivative or fragment thereof having bone anabolic activity, a PTHrP or a derivative or fragment thereof having bone anabolic activity, and abaloparatide or a derivative or fragment thereof having bone anabolic activity.

6. The compound of any one of the preceding claims, wherein the bone anabolic agent is abaloparatide or a derivative or fragment thereof having bone anabolic activity.

7. The compound of claim 1, wherein Z is a tetracycline, a ranelate, a calcium chelator, a metal chelator, a bisphosphonate, or an AOP.

8. The compound of any one of claim 1, 2, 5, or 7, wherein Z is a bisphosphonate selected from the group consisting of monobisphosphonate, tribisphosphonate, and polybisphosphonate.

9. The compound of any one of claims 1-5, wherein Z is a linear chain of amino acid residues.

10. The compound of any one of claims 1-5, wherein Z is a branched chain of amino acid residues.

11. The compound of claim 1, wherein Z is an AOP comprising at least 4 glutamic acid amino acid residues or at least 4 aspartic acid amino acid residues.

12. The compound of any one of claims 1, 2, 5, and 11, wherein Z comprises at least 4 amino acid residues having the same chirality.

13. The compound of any one of claims 1, 2, 5, and 11, wherein Z comprises at least 4 amino acid residues and at least 4 of such amino acid residues has D chirality.

14. The compound of any one of claims 1, 2, 5, and 11, wherein Z comprises at least 4 glutamic acid amino acid residues, at least 4 aspartic acid amino acid residues, or at least 4 glutamic acid amino acid residues and at least 4 aspartic acid amino acid residues.

15. The compound of any one of claims 1, 2, 5, and 11, wherein Z comprises 4 to 20 D-glutamic acid amino acid residues, 4 to 20 D-aspartic acid amino acid residues, or 4 to 20 D-glutamic acid amino acid residues and 4 to 20 D-aspartic acid amino acid residues.

16. The compound of any one of claims 1-5 and 11, wherein Z comprises a mixture of glutamic acid amino acid residues and aspartic acid amino acid residues.

17. The compound of claim 1, wherein Z comprises at least 15 repeating D-glutamic acid amino acid residues (DE15), or at least 20 repeating D-glutamic acid amino acid residues (DE20).

18. The compound of any one of claims 1, 2, 5, and 11, wherein Z is DE10 or DE20.

19. The compound of any one of claims 1, 2, 5, and 11, wherein Z comprises 4 to 75 acidic amino acid residues.

20. The compound of any one of claims 1, 2, 5 and 11, wherein Z comprises 4 to 75 D-glutamic acid amino acid residues.

21. The compound of any one of claims 1, 2, 5, and 11, wherein Z comprises 8 to 30 acidic amino acid residues.

22. The compound of any one of claims 1, 2, 5, and 11, wherein Z comprises 8 to 30 D-glutamic acid amino acid residues.

23. The compound of any one of claims 1, 3-5, 11, and 17, wherein X is abaloparatide or a derivative or fragment thereof having bone anabolic activity and Z is DE20.

24. The compound of any one of claims 1-5, 11, and 17, wherein Y is a non-releasable linker.

25. The compound of any one of claims 1-5, 11, and 17, wherein Y is a non-releasable linker comprising at least one carbon-carbon bond and/or at least one amide bond.

26. The compound of any one of claims 1-5, 11, and 17, wherein Y is a releasable linker.

27. The compound of any one of claims 1-5, 11, and 17, wherein Y is a releasable linker comprising at least one disulfide bond, at least one ester, and/or at least one amide bond.

28. The compound of claim 1, wherein X is abaloparatide or a derivative or fragment thereof having bone anabolic activity, Y is a non-releasable oligopeptide linker, and Z is DE20.

29. The compound of claim 1, wherein X is abaloparatide or a derivative or fragment thereof having bone anabolic activity, Y is a releasable oligopeptide linker comprising at least one protease-specific amide bond, and Z is DE20.

30. The compound of claim 1, wherein the compound has at least 75% sequence identity or more, at least 85% sequence identity or more, at least 90% sequence identity or more, or at least 95% sequence identity or more to SEQ ID NO: 3.

31. The compound of claim 1, wherein the compound has at least 75% sequence identity or more, at least 85% sequence identity or more, at least 90% sequence identity or more, or at least 95% sequence identity or more to SEQ ID NO: 14.

32. The compound of claim 1, wherein the compound has at least 75% sequence identity or more, at least 85% sequence identity or more, at least 90% sequence identity or more, or at least 95% sequence identity or more to SEQ ID NO: 4.

33. A pharmaceutical composition comprising a compound of any one of the preceding claims, or a pharmaceutically acceptable salt thereof.

34. A pharmaceutical composition of claim 33, further comprising a pharmaceutically acceptable carrier or excipient.

35. A method of treating a bone fracture in a patient in need thereof, the method comprising administering to the patient a therapeutically effective amount of a compound of any one of claims 1-32 or a pharmaceutical composition of claim 33 or 34, thereby treating the bone fracture in the patient.

36. The method of claim 35, wherein the patient is susceptible to bone fracture.

37. The method of claim 36, wherein the patient has one or more comorbidities selected from the group consisting of diabetes mellitus, osteoporosis, a maxillofacial injury, a maxillofacial deficiency, and a maxillofacial defect.

38. The method of claim 37, wherein the maxillofacial injury is a maxillofacial fracture.

39. The method of any one of claims 35-37, wherein administering the therapeutically effective amount of the compound or pharmaceutical composition of any one of the preceding claims is by injection, parenteral administration, or enteral administration.

40. The method of claim 39, wherein the injection is subcutaneous.

41. The method of claim 37, further comprising administering a second therapy to the patient for treating the bone fracture or the one or more comorbidities.

42. The method of claim 41, wherein the patient has at least diabetes mellitus and administering the second therapy comprises administering a therapeutically effective amount of insulin to the patient.

43. The method of claim 41, wherein administering the second therapy comprises implantation of hardware or one or more therapeutic compounds at a bone fracture site.

44. The method of claim 35, wherein the therapeutically effective amount of the compound or pharmaceutical composition comprises a concentration of compound of at or between 0.01/kg of patient body weight to 1 mg/kg of patient body weight.

45. The method of claim 35, wherein administering to the patient a therapeutically effective amount of the compound or the pharmaceutical composition is repeated 1-800 times during a course of treatment.

46. The method of claim 35, wherein administering results in a reduction of pain in the patient within three weeks following administration of the therapeutically effective amount of the compound or the pharmaceutical composition.

47. A method of promoting bone growth in a patient in need thereof, the method comprising administering to the patient a therapeutically effective amount of a compound of any one of claims 1-32 or a pharmaceutical composition of claim 33 or 34, thereby increasing a bone mineral density in a bone of the patient as compared to pre-treatment.

48. The method of claim 47, wherein the patient has osteoporosis.

49. The method of claim 48, wherein the increased bone mineral density in the bone occurs at a fracture site.

50. The method of claim 48, wherein the increased bone mineral density in the bone occurs at one or more resorption pits present on the bone prior to the administering step.


SEQUENCE LISTING (21 sequences)

SEQ ID NO: 1 - protein, 86 aa, Artificial Sequence
  modified parathyroid hormone-related protein (PTHrP)
AVSEHQLLHDKGKSIQDLRRRFFLHHLIAEIHTAEIRATSEVSPNSKPSPNTKNHPVRFGSDDEGRYLTQETNKVETYKEQPLKTP

SEQ ID NO: 2 - protein, 34 aa, Artificial Sequence
  abaloparatide, an analog of PTHrP
  SITE (29)..(29): Where “X” is alpha-aminoisobutyric acid
  SITE (29)..(29): where “X” is alpha-aminoisobutyric acid
AVSEHQLLHDKGKSIQDLRRRELLEKLLXKLHTA

SEQ ID NO: 3 - protein, 56 aa, Artificial Sequence
  a conjugate with 10 glutamic acid residues
  SITE (29)..(29): where “X” is methylalanine
AVSEHQLLHDKGKSIQDLRRRELLEKLLXKLHTAEIRATSEVSPNSEEEEEEEEEE

SEQ ID NO: 4 - protein, 65 aa, Artificial Sequence
  a conjugate with 20 glutamic acid residues
  SITE (29)..(29): where “X” is alpha-aminoisobutyric acid
AVSEHQLLHDKGKSIQDLRRRELLEKLLXKLHTAEIRATSEVSPNSEEEEEEEEEEEEEEEEEEE

SEQ ID NO: 5 - protein, 42 aa, Artificial Sequence
  a modified peptide hormone conjugate with 10 glutamic acid residues
  MISC_FEATURE (11)..(12)
HEEEEEEEEEERGASQRWTDYQFFGVPYRPFDPLVAQSTSVD

SEQ ID NO: 6 - protein, 11 aa, Artificial Sequence
  D10-ester-dasatinib
  SITE (1)..(1): where “X” indicates ester-dasatinib
XDDDDDDDDDD

SEQ ID NO: 7 - protein, 18 aa, Artificial Sequence
  a conjugate with 10 glutamic acid residues
  MISC_FEATURE (11)..(12)
HEEEEEEEEEERRETAWA

SEQ ID NO: 8 - protein, 18 aa, Artificial Sequence
  OTHER INFORMATION ITGA conjugated with 10 glutamic acid residues (ITGA5)
  MISC_FEATURE (11)..(12)
HEEEEEEEEEERRETAWA

SEQ ID NO: 9 - protein, 26 aa, Artificial Sequence
  a conjugate with 10 glutamic acid residues
  MISC_FEATURE (11)..(12)
HEEEEEEEEEEKLTWQELYQLKYKGI

SEQ ID NO: 10 - protein, 22 aa, Artificial Sequence
  a conjugate with 10 aspartic acid residues
  MISC_FEATURE (11)..(12)
HDDDDDDDDDDRPKPQQFFGLM

SEQ ID NO: 11 - protein, 65 aa, Artificial Sequence
  a conjugate with 20 glutamic acid residues
  misc_feature (29)..(29): Xaa can be any naturally occurring amino acid
AVSEHQLLHDKGKSIQDLRRRELLEKLLXKLHTAEIRATSEVSPNEEEEEEEEEEEEEEEEEEEE

SEQ ID NO: 12 - protein, 12 aa, Artificial Sequence
  a linker
EIRATSEVSPNS

SEQ ID NO: 13 - protein, 56 aa, Artificial Sequence
  a conjugate with 10 glutamic acid residues
  SITE (47)..(56): where the glutamic acid residues at positions 47-56 have D-chirality
SVSEIQLMHNLGKHLNSMERVEWLRKKLQDVHNFVALGAPLAPRDAEEEEEEEEEE

SEQ ID NO: 14 - protein, 56 aa, Artificial Sequence; identical to SEQ ID NO: 3
  a conjugate with 10 glutamic acid residues
  SITE (29)..(29): where “X” is methylalanine
  SITE (47)..(56): where the glutamic acid residues at positions 47-56 have L-chirality

SEQ ID NO: 15 - protein, 46 aa, Artificial Sequence
  a compound having the X-Y portion of Formula (I)
  SITE (29)..(29): where “X” is alpha-aminoisobutyric acid or mehtylalanine
AVSEHQLLHDKGKSIQDLRRRELLEKLLXKLHTAEIRATSEVSPNS

SEQ ID NO: 16 - protein, 8 aa, Artificial Sequence
  heparin-binding domain of FGF2
YKRSRYTC

SEQ ID NO: 17 - protein, 39 aa, Artificial Sequence
  pituitary adenylate cyclase-activating polypeptide
HSDGIFTDSYSRYRKQMAVKKYLAAVLGKRYKQRVKNKC

SEQ ID NO: 18 - protein, 15 aa, Artificial Sequence
  chemotactic cryptic peptide, derived from the CTX region of collagen type III
YIAGVGGEKSGGFYC

SEQ ID NO: 19 - protein, 30 aa, Artificial Sequence
  casein kinase 2 beta chain
RQIKIWFQNRRMKWKKIPVGESLKDLIDQC

SEQ ID NO: 20 - protein, 16 aa, Artificial Sequence
  osteopontin-derived peptide
DVDVPDGRGDSLAYGC

SEQ ID NO: 21 - protein, 21 aa, Artificial Sequence
  BMP-2 fragment
KIPKASSVPTELSAISTLYLC